(19) United States
(12) Patent Application Publication
(10) Pub. No.: US 2026/0183295 A1
(43) Pub. Date: Jul. 2, 2026

(54) METHODS AND COMPOSITIONS FOR TREATING CANCER

(71) Applicants: Krishnaraj Rajalingam, Nieder-Olm (DE); Onconova Therapeutics, Inc., Newtown, PA (US)

(72) Inventors: Krishnaraj Rajalingam, Nieder-Olm (DE); Steven M. Fruchtman, Newtown, PA (US); Matthew Parris, Newton, PA (US); Mark S. Gelder, Newtown, PA (US); Stephen C. Cosenza, Newtown, PA (US)

(21) Appl. No.: 19/129,423

(22) PCT Filed: Nov. 16, 2023
(86) PCT No.: PCT/US2023/080013

§ 371 Date: May 13, 2025

Related U.S. Application Data
(60) Provisional application No. 63/426,333, filed on Nov. 17, 2022.
(60) Provisional application No. 63/496,339, filed on Apr. 14, 2023.

(51) Int. Cl. A61K 31/519; A61K 31/4196; A61K 31/565; A61K 31/57; A61P 35/00

(52) U.S. Cl. CPC A61K 31/519; A61K 31/4196; A61K 31/565; A61K 31/57; A61P 35/00

(57) ABSTRACT

Disclosed herein is a method of treating breast cancer by administering 8-cyclopentyl-2-((4-(4-methylpiperazin-1-yl)phenyl)amino)-7-oxo-7,8-dihydropyrido[2,3-d]pyrimidine-6-carbonitrile, alone or in combination with a second agent such as aromatase inhibitor or a selective estrogen receptor degrader. Also disclosed herein are combinations of 8-cyclopentyl-2-((4-(4-methylpiperazin-1-yl)phenyl)amino)-7-oxo-7,8-dihydropyrido[2,3-d]pyrimidine-6-carbonitrile and second agent such as aromatase inhibitor or a selective estrogen receptor degrader.

### CROSS-REFERENCE

The present application claims the benefit of U.S. Provisional Application No. 63/426,333, filed Nov. 17, 2022, and U.S. Provisional Application No. 63/496,339, filed Apr. 14, 2023, each of which is incorporated by reference herein in its entirety.

### BACKGROUND

Breast cancer accounts for about 30% of all new female cancers each year. A large subset of breast cancers has alterations in both the phosphatidylinositol-4,5-bisphosphate 3-kinase (PI3K) and mitogen-activated protein kinase (MAPK) pathways.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE DISCLOSURE

In some embodiments, the present disclosure provides methods for treating breast cancer in a subject in need thereof, the method comprising administering to the subject a therapeutically-effective amount of a compound of formula (I):

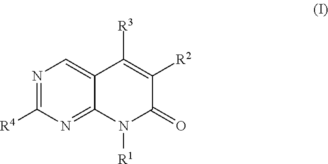

wherein:
   R¹ is alkyl, alkenyl, alkynyl, cycloalkyl, aryl, heteroaryl, or heterocyclyl, each of which is unsubstituted or substituted, or hydrogen;
   R² is alkyl, alkenyl, alkynyl, cycloalkyl, aryl, heteroaryl, heterocyclyl, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or —CN, hydrogen, or halogen;
   R³ is alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —C(O)R⁵, —C(O)OR⁵, —C(O)NR⁵R⁶, —C(═N)NR⁵R⁶, —OR⁵, —SR⁵, —NR⁵R⁶, —NRSC(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or —CN, hydrogen, or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen;
   provided that, when R⁴ is —NR⁵R⁶ and one of R⁵ and R⁶ is pyridyl, then R² is not —C(O)CH₃,
   or a pharmaceutically-acceptable salt thereof,
   wherein the administering is once daily.

In some embodiments, the present disclosure provides methods for treating breast cancer in a subject in need thereof, the method comprising administering to the subject a therapeutically-effective amount of a compound of formula (I):

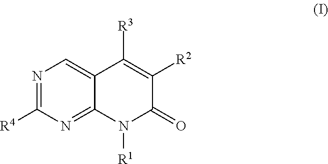

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or —CN, hydrogen, or halogen;
   R³ is alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or —CN, hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen;
   provided that, when R⁴ is —NR⁵R⁶ and one of R⁵ and R⁶ is pyridyl, then R² is not —C(O)CH₃, or a pharmaceutically-acceptable salt thereof,
wherein the administering is a 4-week cycle of: (i) a continuous, three-week period of once-daily administration; and (ii) immediately following the three-week period, one week of no administration.

In some embodiments, the present disclosure provides methods for treating breast cancer in a subject in need thereof, the method comprising administering to the subject a therapeutically-effective amount of a compound of formula (I):

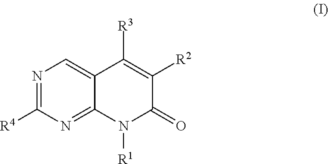

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or —CN, hydrogen, or halogen;
   R³ is alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or —CN, hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen;
   provided that, when R⁴ is —NR⁵R⁶ and one of R⁵ and R⁶ is pyridyl, then R² is not —C(O)CH₃,
   or a pharmaceutically-acceptable salt thereof,
   wherein the subject received a therapy other than the compound for the breast cancer prior to the administering.

In some embodiments, the present disclosure provides a method for treating breast cancer in a subject in need thereof, the method comprising:
(i) administering to the subject a therapeutically-effective amount of a first compound, wherein the first compound is a compound of formula (I):

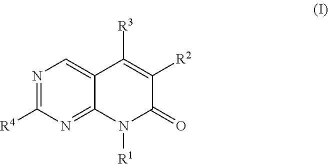

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or —CN, hydrogen, or halogen;
   R⁴ is alkyl, alkenyl, alkynyl, cycloalkyl, aryl, heterocyclyl, heteroaryl, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or —CN, hydrogen, or halogen;
      provided that, when R⁴ is —NR⁵R⁶ and one of R⁵ and R⁶ is pyridyl, then R² is not —C(O)CH₃,
   or a pharmaceutically-acceptable salt thereof; and
(ii) administering to the subject a therapeutically-effective amount of an aromatase inhibitor. In some embodiments, the aromatase inhibitor is letrozole or a pharmaceutically-acceptable salt thereof.

In some embodiments, the present disclosure provides a method for treating breast cancer in a subject in need thereof, the method comprising:
(i) administering to the subject a therapeutically-effective amount of a first compound, wherein the first compound is a compound of formula (I):

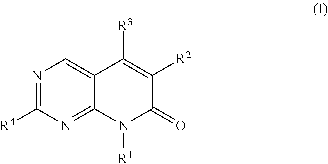

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or —CN, hydrogen, or halogen;
   R⁴ is alkyl, alkenyl, alkynyl, cycloalkyl, aryl, heterocyclyl, heteroaryl, —C(O)R⁵, —C(O)OR⁵, —C(O)NR⁵R⁶, —C(═N)NR⁵R⁶, —OR⁵, —SR⁵, —NR⁵R⁶, —NRSC(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or —CN, hydrogen, or halogen;
      provided that, when R⁴ is —NR⁵R⁶ and one of R⁵ and R⁶ is pyridyl, then R² is not —C(O)CH₃,
   or a pharmaceutically-acceptable salt thereof; and
(ii) administering to the subject a therapeutically-effective amount of a selective estrogen receptor degrader. In some embodiments, the selective estrogen receptor degrader is fulvestrant.

In some embodiments, the present disclosure provides a combination comprising
(i) a compound of formula (I):

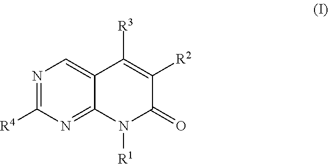

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or —CN, hydrogen, or halogen;
   R³ is C₂-C₆ alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or —CN, hydrogen, or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen,
or a pharmaceutically-acceptable salt thereof; and
(ii) an aromatase inhibitor. In some embodiments, the aromatase inhibitor is letrozole or a pharmaceutically-acceptable salt thereof.

In some embodiments, the present disclosure provides a combination comprising
(i) a compound of formula (I):

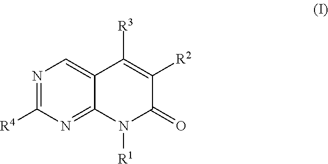

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —C(O)R⁵, —C(O)OR⁵, —C(O)NR⁵R⁶, —C(═N)NR⁵R⁶, —OR⁵, —SR⁵, —NR⁵R⁶, —NRSC(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or —CN, hydrogen, or halogen;
   R³ is C₂-C₆ alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or —CN, hydrogen, or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen,
or a pharmaceutically-acceptable salt thereof; and
(ii) a selective estrogen receptor degrader. In some embodiments, the selective estrogen receptor degrader is fulvestrant.

In some embodiments, the present disclosure provides a method of treating breast cancer in a subject in need thereof, the method comprising orally administering to the subject a solid pharmaceutical composition, the solid pharmaceutical composition comprising 40 mg to 500 mg of a compound that is 8-cyclopentyl-2-((4-(4-methylpiperazin-1-yl)phenyl)amino)-7-oxo-7,8-dihydropyrido[2,3-d]pyrimidine-6-carbonitrile monolactate, wherein the subject received a therapy other than the compound for the breast cancer prior to the administering, wherein the therapy was received after the subject was diagnosed with breast cancer, and wherein the subject has not responded to the therapy prior to the administering; and wherein the administering comprises 3 weeks of once-daily administration.

In some embodiments, the present disclosure provides a method of treating estrogen receptor positive, progesterone receptor positive, human epidermal growth factor 2 (HER)2 (or HER2) positive (or “+”) breast cancer in a subject in need thereof. In some embodiments, the present disclosure provides a method of treating estrogen receptor positive, progesterone receptor positive, human epidermal growth factor 2 (HER)2 negative (or “−”) breast cancer in a subject in need thereof. In some embodiments, the present disclosure provides a method of treating estrogen receptor positive, progesterone receptor negative, human epidermal growth factor 2 (HER)2 positive breast cancer in a subject in need thereof. In some embodiments, the present disclosure provides a method of treating estrogen receptor negative, progesterone receptor negative, human epidermal growth factor 2 (HER)2 positive breast cancer in a subject in need thereof. In some embodiments, the present disclosure provides a method of treating estrogen receptor negative, progesterone receptor negative, human epidermal growth factor 2 (HER)2 negative breast cancer, e.g., triple-negative breast cancer (TNBC) in a subject in need thereof.

In some embodiments, the method comprises orally administering to the subject a solid pharmaceutical composition, the solid pharmaceutical composition comprising 40 mg to 500 mg of a compound that is 8-cyclopentyl-2-((4-(4-methylpiperazin-1-yl)phenyl)amino)-7-oxo-7,8-dihydropyrido[2,3-d]pyrimidine-6-carbonitrile monolactate, wherein the subject received a therapy other than the compound for the breast cancer prior to the administering, wherein the therapy was received after the subject was diagnosed with breast cancer, and wherein the subject has not responded to the therapy prior to the administering; and wherein the administering comprises 3 weeks of once-daily administration.

In some embodiments, the present disclosure provides a method of treating estrogen receptor negative, progesterone receptor negative, human epidermal growth factor 2 (HER)2 positive breast cancer in a subject in need thereof, the method comprising orally administering to the subject a solid pharmaceutical composition, the solid pharmaceutical composition comprising 40 mg to 500 mg of a compound that is 8-cyclopentyl-2-((4-(4-methylpiperazin-1-yl)phenyl)amino)-7-oxo-7,8-dihydropyrido[2,3-d]pyrimidine-6-carbonitrile monolactate, wherein the subject received a therapy other than the compound for the breast cancer prior to the administering, wherein the therapy was received after the subject was diagnosed with breast cancer, and wherein the subject has not responded to the therapy prior to the administering; and wherein the administering comprises 3 weeks of once-daily administration.

In some embodiments, the present disclosure provides a method of treating breast cancer in a subject in need thereof, the method comprising: (i) orally administering to the subject a solid pharmaceutical composition, wherein the solid pharmaceutical composition comprises 40 mg to 500 mg of a compound that is 8-cyclopentyl-2-((4-(4-methylpiperazin-1-yl)phenyl)amino)-7-oxo-7,8-dihydropyrido[2,3-d]pyrimidine-6-carbonitrile monolactate, wherein the subject received a therapy other than the compound for the breast cancer prior to the administering, wherein the therapy was received after the subject was diagnosed with breast cancer, wherein the subject has not responded to the therapy prior to the administering; and wherein the administering comprises 3 weeks of once-daily administration; and (ii) orally administering to the subject a therapeutically-effective amount of letrozole or a pharmaceutically-acceptable salt thereof.

In some embodiments, the present disclosure provides a method of treating breast cancer in a subject in need thereof, the method comprising: (i) orally administering to the subject a solid pharmaceutical composition, wherein the solid pharmaceutical composition comprises 40 mg to 500 mg of a compound that is 8-cyclopentyl-2-((4-(4-methylpiperazin-1-yl)phenyl)amino)-7-oxo-7,8-dihydropyrido[2,3-d]pyrimidine-6-carbonitrile monolactate, wherein the subject received a therapy other than the compound for the breast cancer prior to the administering, wherein the therapy was received after the subject was diagnosed with breast cancer, wherein the subject has not responded to the therapy prior to the administering, and wherein the administering comprises 3 weeks of once-daily administration; and (ii) orally administering to the subject a therapeutically-effective amount of fulvestrant.

In some embodiments, a compound disclosed herein, e.g., 8-cyclopentyl-2-((4-(4-methylpiperazin-1-yl)phenyl)amino)-7-oxo-7,8-dihydropyrido[2,3-d]pyrimidine-6-carbonitrile, shows enhanced efficacy at low nM concentrations against CDK4/6, ARK5/NUAK1, CSF1R, and c-Kit compared to a comparator molecule at comparable nM concentrations. In some embodiments, a compound disclosed herein can be used to treat a disease overexpressing one or more ARK5 and/or NUAKl. In some embodiments, the disease is cancer, e.g., breast cancer, multiple myeloma, hepatocellular carcinoma. In some embodiments, the cancer is metastatic, e.g., metastatic breast cancer. In some embodiments, the subject having the disease is a female, e.g., a postmenopausal female.

In some embodiments, the disease treated by 8-cyclopentyl-2-((4-(4-methylpiperazin-1-yl)phenyl)amino)-7-oxo-7,8-dihydropyrido[2,3-d]pyrimidine-6-carbonitrile can be a hormone receptor positive human epidermal growth factor 2 (HER)2 negative postmenopausal metastatic breast cancer, e.g., resistant to CDK4/6 inhibitors.

### BRIEF DESCRIPTION OF THE DRAWINGS

FIG. 1 shows Compound 1 inhibiting growth of cancer cells that are resistant to Comparator 1 (palbociclib).

FIGS. 2A and 2B show Compound 1 inhibiting wound healing, a proxy for cancer cell migration, more than Comparator 1 based on 24 Hour Migration Assay in Hs578t and MDA-MB-231 cell lines at 1.0 μM concentration (FIG. 2A) and 10.0 μM concentration (FIG. 2B).

FIG. 3 shows the comparative mass spectrometry-based cellular thermal shift assay (CETSA-MS) profiling of MDA-MB-231 intact cells and lysates treated with Compound 1 (at 2 μM and 20 μM concentrations).

FIG. 4 shows the comparative mass spectrometry-based cellular thermal shift assay (CETSA-MS) profiling of MDA-MB-231 intact cells and lysates treated with Comparator 1 (at 2 μM and 20 μM concentrations).

FIGS. 5 and 6 show the phosphoproteome changes of MDA-MB-231 cells treated for 2 hours with Compound 1 compared to Comparator 1.

FIG. 5 shows results of western blot analysis of MDA-MB-231 cells lysates after 2 h treatment with Compound 1 or Comparator 1, from a sample of the cells used for phosphoproteome analysis. Shown is the result for one sample out of three independent replicates.

FIG. 6 demonstrates the Venn diagram representing the number of deregulated phosphopeptides in Comparator 1-treated cells compared to Compound 1-treated cells.

FIGS. 7-12 show BUB1 expression levels in cancer patients based on the bioinformatics data from two human cancer databases: The Cancer Genome Atlas Program (TCGA) and Clinical Proteomic Tumor Analysis Consortium (CPTAC).

FIG. 7 shows BUB1 gene expression in patient samples across 13 TCGA cancer types TCGA.

FIG. 8 shows BUB1 expression in TCGA Uterine Corpus Endometrial Carcinoma (TCGA-UCEC) patient samples across histology grades. Data shows Serous Grade 3 samples show higher BUB1 expression compared to endometrioid tumor samples.

FIG. 9 shows correlation of UCEC patient survival probability over time with high, low, very high, and very low expression of BUB1. Data show UCEC patients showing very high BUB1 expression group has low survival probabilities compared to UCEC patients showing low, high, and very low expression of BUB1.

FIG. 10 shows BUB1 gene expression in different TCGA breast cancer subtype patient samples. Data show Triple Negative subtype (TNBC) has moderately higher median and overall BUB1 expression compared to other breast cancer subtypes.

FIG. 11 shows BUB1 gene expression in various TCGA breast cancer patient samples across American Joint Committee on Cancer (AJCC) stages. Data show that AJCC Stage II samples exhibit higher BUB1 expression compared to other AJCC stages.

FIG. 12 shows correlation of breast cancer patient survival probability over time with high, low, very high, and very low expression of BUBl. Data show that Breast Cancer patient group with very high expression of BUB1 has lower survival probabilities compared to breast cancer patients showing low, high, and very low expression of BUB1.

FIGS. 13-15 show the molecular docking of various CDK4/6 inhibitors—BUB1 interaction profile.

FIG. 13 shows the predicted binding-conformation (left panel) of and binding site (right panel) of Compound 1 to BUB1.

FIG. 14 shows the predicted binding-conformation (left panel) of and binding site (right panel) of Comparator 1 to BUB1.

FIG. 15 shows the predicted binding-conformation (left panel) of and binding site (right panel) of Comparator 3 (abemaciclib) to BUB1.

FIG. 16 shows results of a western blot analysis of total cell lysates of MDA-MB-231 cells for BUB1 protein levels following 48 hour treatment with (i) Comparator 3 (1 μM), (ii) Comparator 1 (5 μM), (iii) Comparator 2 (ribociclib) (5 μM), (iv) SBI-0206965 (10 μM), (v) hydroxychloroquine (15 μM), (vi) Compound 1 alone (5 μM and 1 μM), (vii) Compound 1 (5 μM and 1 μM) in combination with SBI-0206965(10 μM) and (viii) Compound 1 (5 μM and 1 μM) in combination with hydroxychloroquine (15 μM). DMSO treatment and GADPH protein level served as negative controls.

FIG. 17 shows the effects of Compound 1(2.5 μM, 5 μM, and 10 μM), Comparator 1 (5 μM), Comparator 2 (5 μM), and Comparator 4 (bortezomib) (10 μM) on the viability of T-47D, HCC-1937, HS-578T, BT-474, MDA-MB-468, and MD-MB-453 cell lines as measured with MTT assay after 48 hours treatment. DMSO treatment served as negative control.

FIG. 18 shows representative images demonstrating the effects of Compound 1(5 μM), Comparator 1 (5 μM), or Comparator 2 (5 μM), on cultured HS-578T spheroids viability by CellTiter-Glo® 3D Cell Viability Assay after 3 days of treatment. Untreated cells served as negative control.

FIG. 19 shows representative quantification demonstrating the effects of Compound 1 (5 μM), Comparator 1 (5 μM), Comparator 2 (5 μM), Comparator 4 (10 μM), on cultured HS-578T spheroids viability by CellTiter-Glo® 3D Cell Viability Assay after 48 hours of treatment. Untreated cells served as negative control.

FIG. 20 shows a western blot analysis of lysates obtained from MDA-MB-231 cells treated for 24 hours with Compound 1(2.5 μM, 5 μM, and 10 μM), Comparator 1 (2.5 μM, 5 μM, and 10 μM), Comparator 2 (2.5 μM, 5 μM, and 10 μM), or Comparator 4 (10 μM). DMSO treatment served as negative control.

FIG. 21 shows the effects of Compound 1 (5 μM), Comparator 1 (5 μM), or Comparator 2 (5 μM) on the viability of MDA-MB-231 cells treated for 24 hours relative to the viability of MDA-MB-231 cells treated with DMSO for identical time period as measured by MTT assay.

FIG. 22 shows the effects of Compound 1 (5 μM), Comparator 1 (5 μM), Comparator 2 (5 μM), or Comparator 4 (10 μM) 24 hour treatment on Caspase 3/7 activity of MDA-MB-231 cells as measured with Glo assay. Untreated cells and DMSO treated cells served as negative control.

FIG. 23 shows analysis of cell death of MDA-MB-231 cells treated with Compound 1 (5 μM), Comparator 1 (5 μM), Comparator 2 (5 μM), or Comparator 4 (10 μM) for 24 hours as measured by Annexin V/PI staining followed by FACS. Untreated cells and DMSO treated cells served as negative control.

FIG. 24 shows analysis of cell death of MDA-MB-231 cells treated with Compound 1 (5 μM), Comparator 1(5 μM), Comparator 2 (5 μM), or Comparator 4 (10 μM) for 24 hours in the presence or absence of 20 μM Z-VAD-FMK as measured by Annexin V/PI staining followed by FACS. Untreated cells and DMSO treated cells served as negative control.

FIG. 25 shows analysis of cell death of MDA-MB-231 cells treated with Compound 1 (5 μM), Comparator 1 (5 μM), Comparator 2 (5 μM), or Comparator 4 (10 μM) for 24 hours in the presence or absence of 20 μM Z-VAD-FMK as measured by MTT assay. Untreated cells and DMSO treated cells served as negative control.

FIG. 26 shows Caspase3/7 activity of MDA-MBA-231 cells treated for 24 hours with Compound 1 (5 μM), Comparator 1 (5 μM), Comparator 2 (5 μM), or Comparator 4 (10 μM) in the presence or absence of 20 μM Z-VAD-FMK relative to the Caspase3/7 activity of MDA-MBA-231 cells treated DMSO for the same time period as measured with Glo assay.

FIG. 27 shows transmission electron microscopy images of MDA-MB-231 cells treated for 24 hours with DMSO.

FIG. 28 shows transmission electron microscopy images of MDA-MB-231 cells treated for 24 hours with 5 μM Compound 1.

FIG. 29 shows morphological changes of MDA-MB-231 cells after 2 days of treatment with DMSO (top panel) or Compound 1 (5 μM) (2^{nd}, 3^{rd}, and 4^{th} panel from top). Phase contrast (PH) microscopy images show formation of large vacuoles in the cytoplasm, that colocalize with Lysotracker Green staining (bottom panel).

FIG. 30 shows analysis of cell death of MDA-MB-231 cells treated with Compound 1 (5 μM) for 2 days in the presence or absence of bafilomycin A1 as measured by Annexin V/PI staining followed by FACS.

FIG. 31 shows analysis of cell death of MDA-MB-231 cells treated with Comparator 1 (5 μM) for 2 days in the presence or absence of bafilomycin A1 as measured by Annexin V/PI staining followed by FACS.

FIG. 32 shows analysis of cell death of MDA-MB-231 cells treated with 5 μM Compound 1 for 2 days in the presence or absence of bafilomycin A1 as measured by the CytoTox-Glo cytotoxicity assay. Shown are mean RLU values relative to DMSO control.

FIG. 33 shows analysis of cell death of MDA-MB-231 cells treated with 5 μM Comparator 1 for 2 days in the presence or absence of bafilomycin A1 as measured by the CytoTox-Glo cytotoxicity assay. Shown are mean RLU values relative to DMSO control.

FIG. 34 shows Caspase3/7 activity in MDA-MB-231 cells treated with 5 μM Compound 1 for 2 days in the presence or absence of bafilomycin A1 as measured by Glo assay. Shown are relative luminescence unit (RLU) values relative to DMSO control.

FIG. 35 shows Caspase3/7 activity in MDA-MB-231 cells treated with 5 μM Comparator 1 for 2 days in the presence or absence of bafilomycin A1 as measured by Glo assay. Shown are relative luminescence unit (RLU) values relative to DMSO control.

FIG. 36 shows cell viability of mouse PYMT cells treated with Compound 1 (2.5 μM, 5 μM, and 10 μM) or Comparator 1 (5 μM) for 24, 48, or 72 hours as measured by the MTT assay. DMSO treated cells served as negative control.

FIG. 37 shows the percentage of apoptotic PYMT cells treated for 48 hours with Compound 1 (1 μM, 5 μM, and 10 μM), Comparator 1 (5 μM), or Comparator 3 (1 μM), as measured by Annexin V/PI staining followed by FACS.

FIG. 38 shows representative phase contrast microscopy images of PYMT cells treated for 48 hours with DMSO (1 μM, 2.5 μM, and 5 μM), Comparator 1 (5 μM), or Comparator 3 (1 μM) with cells analyzed for the presence of senescence-associated β-galactosidase.

FIG. 39 shows mRNA expression levels of CCL5 following treatment with DMSO, Compound 1 (2.5 μM, 5 μM, and 10 μM) or Comparator 1 (5 μM) for 24, 48, or 72 hours as measured by quantitative-polymerase chain reaction (qPCR).

FIG. 40 shows mRNA expression levels of CXCL10 following treatment with DMSO, Compound 1 (2.5 μM, 5 μM, and 10 μM) or Comparator 1 (5 μM) for 24, 48, or 72 hours as measured by quantitative-polymerase chain reaction (qPCR).

FIG. 41 shows mRNA expression levels of H2D1 following treatment with DMSO, Compound 1 (2.5 μM, 5 μM, and 10 μM) or Comparator 1 (5 μM) for 24, 48, or 72 hours as measured by quantitative-polymerase chain reaction (qPCR).

FIG. 42 shows mRNA expression levels of B2M following treatment with DMSO, Compound 1 (2.5 μM, 5 μM, and 10 μM) or Comparator 1 (5 μM) for 24, 48, or 72 hours as measured by quantitative-polymerase chain reaction (qPCR).

FIG. 43 shows growth and recovery of PYMT cells treated with hydroxychloroquine, SBI-0206965, Compound 1, Comparator 1, and Comparator 3 or combinations thereof following 6 days of treatment. Results show, after 6 days SBI sensitizes only cells treated with Compound 1 or Compound 1 combinations.

FIG. 44 shows the IC50 curve of Compound 1 as determined by Cellular NanoBret Assay.

FIG. 45 shows the IC50 curve of Reference compound APY-69 (29B) as determined by Cellular NanoBret Assay.

FIG. 46 shows comparison of IC50 data of Compound 1, Comparator 1, Comparator 2, and Comparator 3 for CDK16/Cyclin Y as calculated by HotSpot™ Kinase Assay.

FIG. 47 shows curve images obtained from Kinase assays for Compound 1 for CDk4-CyclinD1. The amount of kinase measured by qPCR (Signal; y-axis) was plotted against the corresponding compound concentration in nM in log 10 scale (x-axis). Data points marked with an “x” were not used for Kd determination.

FIG. 48 shows curve images obtained from Kinase assays for Comparator 1 for CDk4-CyclinD1. The amount of kinase measured by qPCR (Signal; y-axis) was plotted against the corresponding compound concentration in nM in log 10 scale (x-axis). Data points marked with an “x” were not used for Kd determination.

FIG. 49 shows curve images obtained from Kinase assays for Comparator 2 for CDk4-CyclinD1. The amount of kinase measured by qPCR (Signal; y-axis) was plotted against the corresponding compound concentration in nM in log 10 scale (x-axis). Data points marked with an “x” were not used for Kd determination.

FIG. 50 shows curve images obtained from Kinase assays for Compound 1 for Compound 1 for Ark5. The amount of kinase measured by qPCR (Signal; y-axis) was plotted against the corresponding compound concentration in nM in log 10 scale (x-axis). Data points marked with an “x” were not used for Kd determination.

FIG. 51 shows curve images obtained from Kinase assays for Compound 1 for Ark5. The amount of kinase measured by qPCR (Signal; y-axis) was plotted against the corresponding compound concentration in nM in log 10 scale (x-axis). Data points marked with an “x” were not used for Kd determination.

The patent or application file contains at least one drawing executed in color. Copies of this patent or patent application publication with colored drawing(s) will be provided by the Office upon request and payment of the necessary fee.

### DETAILED DESCRIPTION OF THE DISCLOSURE

Provided herein are compositions and methods for treating a reproductive organ cancer, e.g., breast cancer, by administering to a subject in need thereof a pharmaceutical composition, the pharmaceutical composition comprising in a unit dosage form a therapeutically-effective amount of a compound described herein (Compound 1 or compound 1 or compound (1) or Compound (1), used interchangeably herein) or a pharmaceutically-acceptable salt thereof. In some embodiments, Compound 1 is 8-cyclopentyl-2-((4-(4-methylpiperazin-1-yl)phenyl)amino)-7-oxo-7,8-dihydropyrido[2,3-d]pyrimidine-6-carbonitrile. In some embodiments, Compound is present is a salt form, e.g., 8-cyclopentyl-2-((4-(4-methylpiperazin-1-yl)phenyl)amino)-7-oxo-7,8-dihydropyrido[2,3-d]pyrimidine-6-carbonitrile monolactate salt (Compound 1 salt).

In some embodiments, the method further comprises administering one or more additional therapeutic agents. In some embodiments, the methods further comprise administering a second pharmaceutical composition comprising in a unit dosage form a therapeutically-effective amount of a second therapeutic agent. In some embodiments, the methods further comprise administering a third pharmaceutical composition comprising in a unit dosage form a therapeutically-effective amount of a third therapeutic agent. In some embodiments, the methods further comprise administering a fourth pharmaceutical composition comprising in a unit dosage form a therapeutically-effective amount of a fourth therapeutic agent. In some embodiments, the breast cancer is an estrogen receptor positive (or “+”), progesterone receptor positive, human epidermal growth factor 2 (HER)2 (or HER2) positive breast cancer. In some embodiments, the breast cancer is an estrogen receptor negative (or “−”), progesterone receptor negative, human epidermal growth factor 2 (HER)2 positive breast cancer. In some embodiments, the breast cancer is an estrogen receptor positive, progesterone receptor negative, human epidermal growth factor 2 (HER)2 positive breast cancer. In some embodiments, the breast cancer is an estrogen receptor positive, progesterone receptor negative, human epidermal growth factor 2 (HER)2 positive breast cancer. In some embodiments, the breast cancer is an estrogen receptor negative, progesterone receptor negative, human epidermal growth factor 2 (HER)2 negative breast cancer. In some embodiments, the breast cancer is an estrogen receptor positive, progesterone receptor positive, human epidermal growth factor 2 (HER)2 negative breast cancer. In some embodiments, the breast cancer is an estrogen receptor positive, progesterone receptor negative, human epidermal growth factor 2 (HER)2 negative breast cancer. In some embodiments, the breast cancer is an estrogen receptor negative, progesterone receptor positive, human epidermal growth factor 2 (HER)2 negative breast cancer. In some embodiments, the breast cancer is an estrogen receptor positive, progesterone receptor positive, human epidermal growth factor 2 (HER)2 positive breast cancer. In some embodiments, the breast cancer is metastatic breast cancer. In some embodiments, the breast cancer is a refractory or recurrent breast cancer. In some embodiments, the breast cancer is associated with expression of BUB1, e.g., very high, high, low, or very expression of BUBl. In some embodiments, the breast cancer does not express BUB1, e.g., no detectable expression of BUB1 compared to a healthy cell. In some embodiments, the breast cancer is associated with expression of BRCA1, e.g., very high, high, low, or very expression of BRCA1. In some embodiments, the breast cancer does not express BRCA1, e.g., no detectable expression of BRCA1 compared to a healthy cell.

### Compounds of the Disclosure

A compound disclosed herein can be of the formula (I):

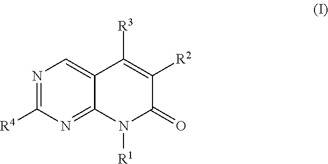

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —C(O)R⁵, —C(O)OR⁵, —C(O)NR⁵R⁶, —C(═N)NR⁵R⁶, —OR⁵, —SR⁵, —NR⁵R⁶, —NRSC(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or —CN, hydrogen, or halogen;
   R⁴ is alkyl, alkenyl, alkynyl, cycloalkyl, aryl, heterocyclyl, heteroaryl, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or —CN, hydrogen, or halogen;
provided that, when R⁴ is —NR⁵R⁶ and one of R⁵ and R⁶ is pyridyl, then R² is not —C(O)CH₃,
or a pharmaceutically-acceptable salt thereof.

A compound disclosed herein can be of the formula:

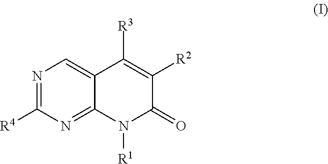

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —C(O)R⁵, —C(O)OR⁵, —C(O)NR⁵R⁶, —C(═N)NR⁵R⁶, —OR⁵, —SR⁵, —NR⁵R⁶, —NRSC(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is C₂-C₆ alkyl, alkenyl, alkynyl, cycloalkyl, aryl, heterocyclyl or heteroaryl, each of which is unsubstituted or substituted, or —CN, hydrogen or halogen;
   R⁴ is alkyl, alkenyl, alkynyl, cycloalkyl, aryl, heterocyclyl, heteroaryl, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or —CN, hydrogen, or halogen,
or a pharmaceutically-acceptable salt thereof.

A compound disclosed herein can be a pharmaceutically-acceptable salt of the formula:

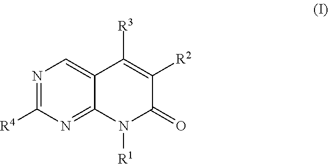

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or —CN, hydrogen, or halogen;
   R⁴ is alkyl, alkenyl, alkynyl, cycloalkyl, aryl, heterocyclyl, heteroaryl, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or —CN, hydrogen, or halogen.

A compound disclosed herein can be a lactate salt of the formula:

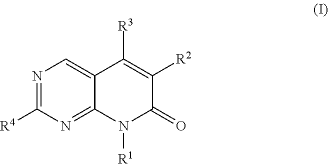

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or —CN, hydrogen, or halogen;
   R⁴ is alkyl, alkenyl, alkynyl, cycloalkyl, aryl, heterocyclyl, heteroaryl, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or —CN, hydrogen or halogen.

In some embodiments, R¹ is cycloalkyl. In some embodiments, R¹ is C₃-C₈ cycloalkyl. In some embodiments, R¹ is an substituted cyclopentyl. In some embodiments, Ri is an unsubstituted cyclopentyl. In some embodiments, R² is CN. In some embodiments, R³ is hydrogen.

In some embodiments, R⁴ is —NR⁵R⁶. In some embodiments, one of R⁵ and R⁶ is hydrogen. In some embodiments, one of R⁵ and R⁶ is phenyl. In some embodiments, one of R⁵ and R⁶ is phenyl substituted with heterocyclyl. In some embodiments, one of R⁵ and R⁶ is phenyl substituted with heterocyclyl, wherein the heterocyclyl contains at least one ring nitrogen atom. In some embodiments, one of R⁵ and R⁶ is phenyl substituted with C₃-C₈ heterocyclyl. In some embodiments, one of R⁵ and R⁶ is phenyl substituted with C₆ heterocyclyl. In some embodiments, one of R⁵ and R⁶ is phenyl substituted with piperazinyl, wherein the piperazinyl is unsubstituted or substituted. In some embodiments, one of R⁵ and R⁶ is phenyl substituted with piperazinyl, wherein the piperazinyl is substituted with an alkyl. In some embodiments, one of R⁵ and R⁶ is phenyl substituted with 4-methyl piperazinyl.

In some embodiments, R⁴ is

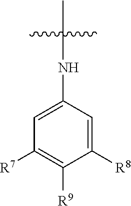

wherein:
   R⁷ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen;
   R⁸ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen; and
   R⁹ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen.

In some embodiments, R⁷ is hydrogen. In some embodiments, R⁸ is hydrogen. In some embodiments, R⁹ is unsubstituted or substituted heterocyclyl. In some embodiments, R⁹ is unsubstituted or substituted piperazinyl. In some embodiments, R⁹ is piperazinyl substituted with alkyl. In some embodiments, R⁹ is 4-methyl piperazinyl.

In some embodiments, the compound is a compound of formula (II):

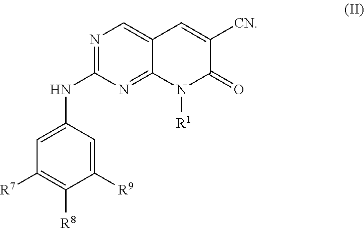

In some embodiments, the compound is a compound of formula (III):

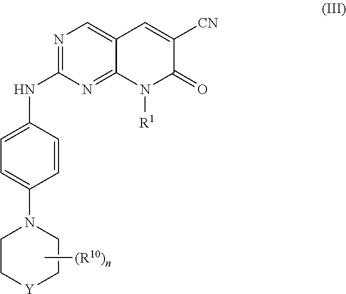

wherein:
   Y is O, S, or NR¹¹;
   each R¹⁰ is independently alkyl, alkenyl, alkynyl, cycloalkyl, aryl, heterocyclyl, heteroaryl, —OR⁵, —SR⁵, or —NR⁵R⁶, each of which is unsubstituted or substituted;
   R¹¹ is alkyl, alkenyl, alkynyl, cycloalkyl, aryl, heterocyclyl, or heteroaryl, each of which is independently substituted or unsubstituted, or hydrogen or halogen; and
   n is 0, 1, 2, 3, 4, 5, 6, 7, or 8.

In some embodiments, R¹ is cycloalkyl. In some embodiments, R¹ is C₃-C₈ cycloalkyl. In some embodiments, R¹ is an unsubstituted cyclopentyl. In some embodiments, R¹ is a substituted cyclopentyl.

In some embodiments, Y is NR¹¹. In some embodiments, R¹¹ is alkyl. In some embodiments, R¹¹ is methyl. In some embodiments, n is 0.

In some embodiments, Y is NR¹¹. In some embodiments, R¹¹ is alkyl. In some embodiments, R¹¹ is methyl. In some embodiments, n is 0.

In some embodiments, the compound is of the formula (1):

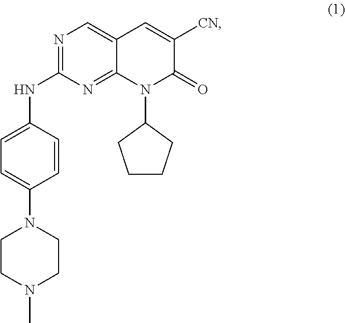 [SMILES: CN1CCN(c2ccc(Nc3ncc4cc(C#N)c(=O)n(C5CCCC5)c4n3)cc2)CC1]

8-cyclopentyl-2-((4-(4-methylpiperazin-1-yl)phenyl)amino)-7-oxo-7,8-dihydropyrido[2,3-d]pyrimidine-6-carbonitrile, or a pharmaceutically-acceptable salt thereof.

In some embodiments, the compound is in the form of a salt formed by combining a compound with lactic acid. In some embodiments, a compound disclosed herein is 8-cyclopentyl-2-((4-(4-methylpiperazin-1-yl)phenyl)amino)-7-oxo-7,8-dihydropyrido[2,3-d]pyrimidine-6-carbonitrile monolactate.

Several moieties described herein can be substituted or unsubstituted. Non-limiting examples of optional substituents include hydroxyl groups, sulfhydryl groups, halogens, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, ureido groups, epoxy groups, and ester groups.

Non-limiting examples of alkyl groups include straight, branched, and cyclic alkyl groups. An alkyl group can be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

Non-limiting examples of straight alkyl groups include methyl, ethyl, propyl, butyl, pentyl, hexyl, heptyl, octyl, nonyl, and decyl.

Branched alkyl groups include any straight alkyl group substituted with any number of alkyl groups. Non-limiting examples of branched alkyl groups include isopropyl, isobutyl, sec-butyl, and t-butyl.

Non-limiting examples of substituted alkyl groups includes hydroxymethyl, chloromethyl, trifluoromethyl, aminomethyl, 1-chloroethyl, 2-hydroxyethyl, 1,2-difluoroethyl, and 3-carboxypropyl.

Non-limiting examples of cyclic alkyl groups include cyclopropyl, cyclobutyl, cyclopentyl, cyclohexyl, cycloheptlyl, and cyclooctyl groups. Cyclic alkyl groups also include fused-, bridged-, and spiro-bicycles and higher fused-, bridged-, and spiro-systems. A cyclic alkyl group can be substituted with any number of straight, branched, or cyclic alkyl groups. Non-limiting examples of cyclic alkyl groups include cyclopropyl, 2-methyl-cycloprop-1-yl, cycloprop-2-en-1-yl, cyclobutyl, 2,3-dihydroxycyclobut-1-yl, cyclobut-2-en-1-yl, cyclopentyl, cyclopent-2-en-1-yl, cyclopenta-2,4-dien-1-yl, cyclohexyl, cyclohex-2-en-1-yl, cycloheptyl, cyclooctanyl, 2,5-dimethylcyclopent-1-yl, 3,5-dichlorocyclohex-1-yl, 4-hydroxycyclohex-1-yl, 3,3,5-trimethylcyclohex-1-yl, octahydropentalenyl, octahydro-1H-indenyl, 3a,4,5,6,7,7a-hexahydro-3H-inden-4-yl, decahydroazulenyl, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

Non-limiting examples of alkenyl and alkenylene groups include straight, branched, and cyclic alkenyl groups. The olefin or olefins of an alkenyl group can be, for example, E, Z, cis, trans, terminal, or exo-methylene. An alkenyl or alkenylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkenyl and alkenylene groups include ethenyl, prop-1-en-1-yl, isopropenyl, but-1-en-4-yl; 2-chloroethenyl, 4-hydroxybuten-1-yl, 7-hydroxy-7-methyloct-4-en-2-yl, and 7-hydroxy-7-methyloct-3,5-dien-2-yl.

Non-limiting examples of alkynyl or alkynylene groups include straight, branched, and cyclic alkynyl groups. The triple bond of an alkylnyl or alkynylene group can be internal or terminal. An alkylnyl or alkynylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkynyl or alkynylene groups include ethynyl, prop-2-yn-1-yl, prop-i-yn-1-yl, and 2-methyl-hex-4-yn-1-yl; 5-hydroxy-5-methylhex-3-yn-1-yl, 6-hydroxy-6-methylhept-3-yn-2-yl, and 5-hydroxy-5-ethylhept-3-yn-1-yl.

A halo-alkyl group can be any alkyl group substituted with any number of halogen atoms, for example, fluorine, chlorine, bromine, and iodine atoms. A halo-alkenyl group can be any alkenyl group substituted with any number of halogen atoms. A halo-alkynyl group can be any alkynyl group substituted with any number of halogen atoms.

An alkoxy group can be, for example, an oxygen atom substituted with any alkyl, alkenyl, or alkynyl group. An ether or an ether group comprises an alkoxy group. Non-limiting examples of alkoxy groups include methoxy, ethoxy, propoxy, isopropoxy, and isobutoxy.

An aryl group can be heterocyclic or non-heterocyclic. An aryl group can be monocyclic or polycyclic. An aryl group can be substituted with any number of substituents described herein, for example, hydrocarbyl groups, alkyl groups, alkoxy groups, and halogen atoms. Non-limiting examples of aryl groups include phenyl, toluyl, naphthyl, pyrrolyl, pyridyl, imidazolyl, thiophenyl, and furyl. Non-limiting examples of substituted aryl groups include 3,4-dimethylphenyl, 4-tert-butylphenyl, 4-cyclopropylphenyl, 4-diethylaminophenyl, 4-(trifluoromethyl)phenyl, 4-(difluoromethoxy)-phenyl, 4-(trifluoromethoxy)phenyl, 3-chlorophenyl, 4-chlorophenyl, 3,4-dichlorophenyl, 2-fluorophenyl, 2-chlorophenyl, 2-iodophenyl, 3-iodophenyl, 4-iodophenyl, 2-methylphenyl, 3-fluorophenyl, 3-methylphenyl, 3-methoxyphenyl, 4-fluorophenyl, 4-methylphenyl, 4-methoxyphenyl, 2,3-difluorophenyl, 3,4-difluorophenyl, 3,5-difluorophenyl, 2,3-dichlorophenyl, 3,4-dichlorophenyl, 3,5-dichlorophenyl, 2-hydroxyphenyl, 3-hydroxyphenyl, 4-hydroxyphenyl, 2-methoxyphenyl, 3-methoxyphenyl, 4-methoxyphenyl, 2,3-dimethoxyphenyl, 3,4-dimethoxyphenyl, 3,5-dimethoxyphenyl, 2,4-difluorophenyl, 2,5-difluorophenyl, 2,6-difluorophenyl, 2,3,4-trifluorophenyl, 2,3,5-trifluorophenyl, 2,3,6-trifluorophenyl, 2,4,5-trifluorophenyl, 2,4,6-trifluorophenyl, 2,4-dichlorophenyl, 2,5-dichlorophenyl, 2,6-dichlorophenyl, 3,4-dichlorophenyl, 2,3,4-trichlorophenyl, 2,3,5-trichlorophenyl, 2,3,6-trichlorophenyl, 2,4,5-trichlorophenyl, 3,4,5-trichlorophenyl, 2,4,6-trichlorophenyl, 2,3-dimethylphenyl, 2,4-dimethylphenyl, 2,5-dimethylphenyl, 2,6-dimethylphenyl, 2,3,4-trimethylphenyl, 2,3,5-trimethylphenyl, 2,3,6-trimethylphenyl, 2,4,5-trimethylphenyl, 2,4,6-trimethylphenyl, 2-ethylphenyl, 3-ethylphenyl, 4-ethylphenyl, 2,3-diethylphenyl, 2,4-diethylphenyl, 2,5-diethylphenyl, 2,6-diethylphenyl, 3,4-diethylphenyl, 2,3,4-triethylphenyl, 2,3,5-triethylphenyl, 2,3,6-triethylphenyl, 2,4,5-triethylphenyl, 2,4,6-triethylphenyl, 2-isopropylphenyl, 3-isopropylphenyl, and 4-isopropylphenyl.

Non-limiting examples of substituted aryl groups include 2-aminophenyl, 2-(N-methylamino)phenyl, 2-(N,N-dimethylamino)phenyl, 2-(N-ethylamino)phenyl, 2-(N,N-diethylamino)phenyl, 3-aminophenyl, 3-(N-methylamino)phenyl, 3-(N,N-dimethylamino)phenyl, 3-(N-ethylamino)phenyl, 3-(N,N-diethylamino)phenyl, 4-aminophenyl, 4-(N-methylamino)phenyl, 4-(N,N-dimethylamino)phenyl, 4-(N-ethylamino)phenyl, and 4-(N,N-diethylamino)phenyl.

A heterocycle can be any ring containing a ring atom that is not carbon, for example, N, O, S, P, Si, B, or any other heteroatom. A heterocycle can be substituted with any number of substituents, for example, alkyl groups and halogen atoms. A heterocycle can be aromatic (heteroaryl) or non-aromatic. Non-limiting examples of heterocycles include pyrrole, pyrrolidine, pyridine, piperidine, succinamide, maleimide, morpholine, imidazole, thiophene, furan, tetrahydrofuran, pyran, and tetrahydropyran.

Non-limiting examples of heterocycles (heterocyclyl) include: heterocyclic units having a single ring containing one or more heteroatoms, non-limiting examples of which include, diazirinyl, aziridinyl, azetidinyl, pyrazolidinyl, imidazolidinyl, oxazolidinyl, isoxazolinyl, thiazolidinyl, isothiazolinyl, oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl, 2,3,4,5-tetrahydro-1H-azepinyl, 2,3-dihydro-1H-indole, and 1,2,3,4-tetrahydroquinoline; and ii) heterocyclic units having 2 or more rings one of which is a heterocyclic ring, non-limiting examples of which include hexahydro-1H-pyrrolizinyl, 3a,4,5,6,7,7a-hexahydro-1H-benzo[d]imidazolyl, 3a,4,5,6,7,7a-hexahydro-1H-indolyl, 1,2,3,4-tetrahydroquinolinyl, and decahydro-1H-cycloocta[b]pyrrolyl.

Non-limiting examples of heteroaryl include: i) heteroaryl rings containing a single ring, non-limiting examples of which include, 1,2,3,4-tetrazolyl, [1,2,3]triazolyl, [1,2,4]triazolyl, triazinyl, thiazolyl, 1H-imidazolyl, oxazolyl, isoxazolyl, isothiazolyl, furanyl, thiophenyl, pyrimidinyl, 2-phenylpyrimidinyl, pyridinyl, 3-methylpyridinyl, and 4-dimethylaminopyridinyl; and ii) heteroaryl rings containing 2 or more fused rings one of which is a heteroaryl ring, non-limiting examples of which include: 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 4,5,6,7-tetrahydro-1-H-indolyl, quinoxalinyl, quinazolinyl, quinolinyl, 8-hydroxy-quinolinyl, and isoquinolinyl.

Any compound herein can be purified. A compound herein can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

### Pharmaceutically Acceptable Salts

The method disclosed herein provides the use of pharmaceutically-acceptable salts of any compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base.

Acid addition salts can arise from the addition of an acid to a compound disclosed herein. In some embodiments, the acid is organic. In some embodiments, the acid is inorganic. In some embodiments, the acid is lactic acid, salicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, maleic acid, hydrochloric acid, hydrobromic acid, hydroiodic acid, nitric acid, nitrous acid, sulfuric acid, sulfurous acid, a phosphoric acid, or isonicotinic acid. In some embodiments, the salt is an acid addition salt with lactic acid. In some embodiments, the salt is an acid addition salt of 8-cyclopentyl-2-((4-(4-methylpiperazin-1-yl)phenyl)amino)-7-oxo-7,8-dihydropyrido[2,3-d]pyrimidine-6-carbonitrile with lactic acid.

In some embodiments, the salt is a lactate salt, a salicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a methanesulfonate salt, an ethanesulfonate salt, a benzenesulfonate salt, a p-toluenesulfonate salt, a citrate salt, an oxalate salt, a maleate salt, hydrochloride salt, a hydrobromide salt, a hydroiodide salt, a nitrate salt, a nitrite salt, a sulfate salt, a sulfite salt, a phosphate salt, isonicotinate salt. In some embodiments, the salt is a lactate salt. In some embodiments, the salt is a monolactate salt. In some embodiments, the compound is 8-cyclopentyl-2-((4-(4-methylpiperazin-1-yl)phenyl)amino)-7-oxo-7,8-dihydropyrido[2,3-d]pyrimidine-6-carbonitrile monolactate.

Metal salts can arise from the addition of an inorganic base to a compound disclosed herein. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

### Therapeutic Methods

Phosphoinositide 3-kinase (PI3 kinase or PI3K), protein kinase B (AKT), and mitogen-activated protein kinase (MAPK) pathways are central to many signaling pathways and are often de-regulated in cancers, including breast cancer. In some embodiments, a compound described herein can down-modulate these kinase pathways, or a portion thereof, for example, cyclin-dependent kinases (CDK). In some embodiments, overexpression of CDK, e.g., CDK 4/6 causes cell-cycle deregulation in cancers. In some embodiments, modulation of kinase pathways can result in the obstruction of proliferation signal receipt in cells, thus arresting tumor growth.

In some embodiments, a compound described herein can be an inhibitor of one or more of cyclin-dependent kinase protein(s), e.g., CDK4, CDK6, CDK4/6. In some embodiments, the cyclin dependent kinase is CDK1, CDK2, CDK3, CDK4, CDK5, CDK6, CDK7, CDK8, CDK9, CDK9, CDK10, CDK 11, CDK12, or CDK13.

In some embodiments, a compound described herein can be an inhibitor of the cyclin-dependent kinase proteins CDK4. In some embodiments, a compound described herein can be an inhibitor of CDK6. In some embodiments, a compound described herein can be an inhibitor of CDK 4/6. In some embodiments, a compound described herein is an inhibitor of the AMPK-related protein kinase 5 (ARK5 or NUAK1) protein. ARK5 regulates Atk-dependent cell survival and migration (e.g., formation of metastases) through inhibition of cellular metabolism. ARK5 overexpression is found in multiple tumors and is associated with poor prognosis in metastatic breast cancer, multiple myeloma, and hepatocellular carcinoma. In some embodiments, inhibition of ARK5 induces cell death through PI3K/AKT/mTOR pathway. In some embodiments, a combination of CDK and ARK5 inhibitors has a synergistic effect on cancer cells by simultaneously inhibiting cell cycle (cytostatic) and cellular metabolism (cytotoxic) through CDK and ARK5, respectively.

In some embodiments, the inclusion of ARK5 in the functional activity profile of a compound described herein overcomes the emergence of resistance to CDK4/6 inhibitors due to the loss of retinoblastoma function and C-Myc overexpression. C-Myc expression is dependent on ARK5 activity and is involved in the pathogenesis of certain tumors. In some embodiments, the dual inhibitory effect of a compound described herein provides a therapeutic strategy to optimize efficacy of CDK 4/6 inhibition and reduce emergence of resistance.

### Diseases/Disorders

The present disclosure provides a method for the use of a compound disclosed herein, for example, for treating cancer.

A method disclosed herein can be used to treat, for example, an infection, e.g., viral infection, e.g., HIV infection, an immune disorder, e.g., autoimmune disorder, an inflammatory disease, e.g., a chronic inflammatory disorder, an infectious disease, a hematological disease, a proliferative disease, e.g., a cancer, a solid tumor, or a liquid tumor.

In some embodiments, the proliferative disease is a carcinoma of the bladder, breast, colon (e.g., colorectal carcinomas such as colon adenocarcinoma and colon adenoma), kidney, epidermis, liver, lung, for example adenocarcinoma, small cell lung cancer and non-small cell lung carcinomas, esophagus, gall bladder, ovary, pancreas e.g., exocrine pancreatic carcinoma, stomach, cervix, thyroid, nose, head and neck, prostate, or skin, for example squamous cell carcinoma; a hematopoietic tumor of lymphoid lineage, for example leukemia, acute lymphocytic leukemia, chronic lymphocytic leukemia, B-cell lymphoma (such as diffuse large B cell lymphoma), T-cell lymphoma, multiple myeloma, mantle cell lymphoma, Hodgkin's lymphoma, non-Hodgkin's lymphoma, hairy cell lymphoma, or Burkett's lymphoma; a hematopoietic tumor of myeloid lineage, for example acute and chronic myelogenous leukemias, myelodysplastic syndrome, or promyelocytic leukemia; thyroid follicular cancer; a tumor of mesenchymal origin, for example fibrosarcoma or rhabdomyosarcoma; a tumor of the central or peripheral nervous system, for example astrocytoma, neuroblastoma, glioma or schwannoma; melanoma; seminoma; teratocarcinoma; osteosarcoma; xeroderma pigmentosum; keratoacanthoma; thyroid follicular cancer; or Kaposi's sarcoma.

In some embodiments, the cancer is a cancer sensitive to inhibition of one or more cyclin dependent kinases, e.g., CDK4 sensitive cancer, CDK6 sensitive cancer, CDK4/6 sensitive cancer.

In some embodiments, the cancer is associated with dysregulation of a gene, e.g., overexpression or underexpression of a gene, e.g., BUB1, BRCA. In some embodiments, the cancer is associated with dysregulation of a transcription factor, e.g., overexpression or underexpression of a transcription factor, e.g., MYC, E2F.

In some embodiments, the cancer is a cancer comprising cancer stem cells (CSCs), e.g., breast cancer. In some embodiments, the cancer is a refractory cancer. In some embodiments, the cancer is a relapsed cancer. In some embodiments, the cancer is resistant and/or non-responsive to a first line of therapy. In some embodiments, the cancer is associated with poor prognosis and/or low survival probability.

In some embodiments, the cancer is a cancer of a reproductive organ. In some embodiments, the reproductive organ cancer is a breast cancer, ovarian cancer, e.g., serous ovarian cancer, e.g., low grade serous ovarian cancer, endometrial cancer. In some embodiments, the breast cancer is a primary breast cancer. In some embodiments, the breast cancer is a secondary breast cancer. In some embodiments, the breast cancer is a metastatic breast cancer. In some embodiments, the breast cancer is hormone receptor positive. In some embodiments, the breast cancer is estrogen receptor positive. In some embodiments, the breast cancer is estrogen receptor negative. In some embodiments, the breast cancer is progesterone receptor positive. In some embodiments, the breast cancer is progesterone receptor negative. In some embodiments, the breast cancer is HER2 receptor negative. In some embodiments, the breast cancer is HER2 receptor positive. In some embodiments, the breast cancer is estrogen receptor positive and progesterone receptor positive. In some embodiments, the breast cancer is estrogen receptor positive and progesterone receptor negative. In some embodiments, the breast cancer is estrogen receptor negative and progesterone receptor positive. In some embodiments, the breast cancer is estrogen receptor negative and progesterone receptor negative. In some embodiments, the breast cancer is an estrogen receptor positive and HER2 receptor positive. In some embodiments, the breast cancer is an estrogen receptor negative and HER2 receptor positive. In some embodiments, the breast cancer is an estrogen receptor positive and HER2 receptor negative. In some embodiments, the breast cancer is an estrogen receptor negative and HER2 receptor negative. In some embodiments, the breast cancer is a progesterone receptor positive and HER2 receptor positive. In some embodiments, the breast cancer is a progesterone receptor negative and HER2 receptor positive. In some embodiments, the breast cancer is a progesterone receptor positive and HER2 receptor negative. In some embodiments, the breast cancer is a progesterone receptor negative and HER2 receptor negative. In some embodiments, the breast cancer is an estrogen receptor positive, progesterone receptor positive, and HER2 receptor positive. In some embodiments, the breast cancer is an estrogen receptor negative, progesterone receptor negative, and HER2 receptor positive. In some embodiments, the breast cancer is an estrogen receptor positive, progesterone receptor negative, and HER2 receptor positive. In some embodiments, the breast cancer is an estrogen receptor negative, progesterone receptor positive, and HER2 receptor positive. In some embodiments, the breast cancer is estrogen receptor positive, progesterone receptor positive, and HER2 receptor negative. In some embodiments, the breast cancer is estrogen receptor positive, progesterone receptor negative, and HER2 receptor negative. In some embodiments, the breast cancer is estrogen receptor negative, progesterone receptor positive, and HER2 receptor negative. In some embodiments, the breast cancer is estrogen receptor negative, progesterone receptor negative, and HER2 receptor negative. In some embodiments, the breast cancer is triple-negative breast cancer (TNBC). In some embodiments, the breast cancer is triple-positive breast cancer. In some embodiments, the breast cancer is a BRCA positive. In some embodiments, the breast cancer shows BUB1 expression, e.g., very high, high, low, or very low expressions of BUB1. In some embodiments, the breast cancer does not show BUB1 expression, e.g., detectable BUB1 expression. In some embodiments, the breast cancer showing high expression of BUB1 is associated with low survival probability. In some embodiments, the breast cancer is a refractory and/or relapsed breast cancer.

Non-limiting examples of tumors that are treatable by a combination of a compound described herein can include solid tumors, solid tumors that are refractory to prior treatment with conventional chemotherapy, and solid tumors that respond to initial chemotherapy but subsequently relapsed. In some embodiments, the tumor is a breast cancer tumor. In some embodiments, the tumor is a metastatic breast cancer tumor. In some embodiments, the breast cancer tumor is hormone receptor positive. In some embodiments, the breast cancer tumor is estrogen receptor positive. In some embodiments, the breast cancer tumor is estrogen receptor negative. In some embodiments, the breast cancer tumor is progesterone receptor positive. In some embodiments, the breast cancer tumor is progesterone receptor negative. In some embodiments, the breast cancer tumor is HER2 receptor negative. In some embodiments, the breast cancer tumor is estrogen receptor positive and progesterone receptor positive. In some embodiments, the breast cancer tumor is estrogen receptor positive and progesterone receptor negative. In some embodiments, the breast cancer tumor is estrogen receptor negative and progesterone receptor positive. In some embodiments, the breast cancer tumor is estrogen receptor negative and progesterone receptor negative. In some embodiments, the breast cancer tumor is estrogen receptor positive, progesterone receptor positive, and HER2 receptor negative. In some embodiments, the breast cancer tumor is estrogen receptor positive, progesterone receptor negative, and HER2 receptor negative. In some embodiments, the breast cancer tumor is estrogen receptor negative, progesterone receptor positive, and HER2 receptor negative. In some embodiments, the breast cancer tumor is estrogen receptor negative, progesterone receptor negative, and HER2 receptor negative. In some embodiments, the breast cancer tumor is TNBC. In some embodiments, the breast cancer tumor is a BRCA positive. In some embodiments, the breast cancer tumor shows BUB1 expression, e.g., very high, high, low, or very low expressions of BUB1. In some embodiments, the breast cancer tumor does not show BUB1 expression, e.g., detectable BUB1 expression. In some embodiments, the breast cancer tumor showing high expression of BUB1 is associated with low survival probability. In some embodiments, the breast cancer tumor is a refractory and/or relapsed.

A tumor response due to a method herein can be measured based on the Response Evaluation Criteria in Solid Tumors (RECIST) classification of responses. To use RECIST requires at least one tumor that can be measured on x-rays, CT scans, or MRI scans. RECIST assigns four categories of response: complete response (CR), a partial response (PR), progressive disease (PD), and stable disease (SD). Key features of the RECIST include definitions of minimum size of measurable lesions, instructions on how many lesions to follow, and the use of unidimensional, rather than bidimensional, measures for overall evaluation.

In some embodiments, the present disclosure provides a method of treating breast cancer in a subject in need thereof, the method comprising administering to the subject a therapeutically-effective amount of a compound of formula (I), or a pharmaceutically-acceptable salt thereof. In some embodiments, the present disclosure provides a method of treating breast cancer in a subject in need thereof, the method comprising administering to the subject a therapeutically-effective amount of 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof. In some embodiments, the present disclosure provides a method of treating breast cancer in a subject in need thereof, the method comprising administering to the subject a therapeutically-effective amount of 8-cyclopentyl-2-((4-(4-methylpiperazin-1-yl)phenyl)amino)-7-oxo-7,8-dihydropyrido[2,3-d]pyrimidine-6-carbonitrile monolactate.

In some embodiments, the breast cancer is a hormone receptor positive breast cancer. In some embodiments, the present disclosure provides a method of treating hormone receptor positive breast cancer in a subject in need thereof, the method comprising administering to the subject a therapeutically-effective amount of a compound of formula (I), or a pharmaceutically-acceptable salt thereof. In some embodiments, the present disclosure provides a method of treating hormone receptor positive breast cancer in a subject in need thereof, the method comprising administering to the subject a therapeutically-effective amount of 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof. In some embodiments, the present disclosure provides a method of treating hormone receptor positive breast cancer in a subject in need thereof, the method comprising administering to the subject a therapeutically-effective amount of 8-cyclopentyl-2-((4-(4-methylpiperazin-1-yl)phenyl)amino)-7-oxo-7,8-dihydropyrido[2,3-d]pyrimidine-6-carbonitrile monolactate.

In some embodiments, the breast cancer is an estrogen receptor positive breast cancer. In some embodiments, the present disclosure provides a method of treating estrogen receptor positive breast cancer in a subject in need thereof, the method comprising administering to the subject a therapeutically-effective amount of a compound of formula (I), or a pharmaceutically-acceptable salt thereof. In some embodiments, the present disclosure provides a method of treating estrogen receptor positive breast cancer in a subject in need thereof, the method comprising administering to the subject a therapeutically-effective amount of 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof. In some embodiments, the present disclosure provides a method of treating estrogen receptor positive breast cancer in a subject in need thereof, the method comprising administering to the subject a therapeutically-effective amount of 8-cyclopentyl-2-((4-(4-methylpiperazin-1-yl)phenyl)amino)-7-oxo-7,8-dihydropyrido[2,3-d]pyrimidine-6-carbonitrile monolactate.

In some embodiments, the breast cancer is a progesterone receptor positive breast cancer. In some embodiments, the present disclosure provides a method of treating progesterone receptor positive breast cancer in a subject in need thereof, the method comprising administering to the subject a therapeutically-effective amount of a compound of formula (I), or a pharmaceutically-acceptable salt thereof. In some embodiments, the present disclosure provides a method of treating progesterone receptor positive breast cancer in a subject in need thereof, the method comprising administering to the subject a therapeutically-effective amount of 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof. In some embodiments, the present disclosure provides a method of treating progesterone receptor positive breast cancer in a subject in need thereof, the method comprising administering to the subject a therapeutically-effective amount of 8-cyclopentyl-2-((4-(4-methylpiperazin-1-yl)phenyl)amino)-7-oxo-7,8-dihydropyrido[2,3-d]pyrimidine-6-carbonitrile monolactate.

In some embodiments, the breast cancer is a human epidermal growth factor receptor 2 (HER2) negative (HER2-) breast cancer. In some embodiments, the present disclosure provides a method of treating human epidermal growth factor receptor 2 (HER2) negative breast cancer in a subject in need thereof, the method comprising administering to the subject a therapeutically-effective amount of a compound of formula (I), or a pharmaceutically-acceptable salt thereof. In some embodiments, the present disclosure provides a method of treating human epidermal growth factor receptor 2 (HER2) positive (HER2+) breast cancer in a subject in need thereof, the method comprising administering to the subject a therapeutically-effective amount of 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof. In some embodiments, the present disclosure provides a method of treating human epidermal growth factor receptor 2 (HER2) positive breast cancer in a subject in need thereof, the method comprising administering to the subject a therapeutically-effective amount of 8-cyclopentyl-2-((4-(4-methylpiperazin-1-yl)phenyl)amino)-7-oxo-7,8-dihydropyrido[2,3-d]pyrimidine-6-carbonitrile monolactate.

In some embodiments, the breast cancer is a human epidermal growth factor receptor 2 (HER2) positive breast cancer. In some embodiments, the present disclosure provides a method of treating human epidermal growth factor receptor 2 (HER2) positive breast cancer in a subject in need thereof, the method comprising administering to the subject a therapeutically-effective amount of a compound of formula (I), or a pharmaceutically-acceptable salt thereof. In some embodiments, the present disclosure provides a method of treating human epidermal growth factor receptor 2 (HER2) positive breast cancer in a subject in need thereof, the method comprising administering to the subject a therapeutically-effective amount of 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof. In some embodiments, the present disclosure provides a method of treating human epidermal growth factor receptor 2 (HER2) positive breast cancer in a subject in need thereof, the method comprising administering to the subject a therapeutically-effective amount of 8-cyclopentyl-2-((4-(4-methylpiperazin-1-yl)phenyl)amino)-7-oxo-7,8-dihydropyrido[2,3-d]pyrimidine-6-carbonitrile monolactate.

In some embodiments, the breast cancer is a hormone receptor positive, human epidermal growth factor receptor 2 (HER2) positive breast cancer. In some embodiments, the present disclosure provides a method of treating hormone receptor positive, human epidermal growth factor receptor 2 (HER2) positive breast cancer in a subject in need thereof, the method comprising administering to the subject a therapeutically-effective amount of a compound of formula (I), or a pharmaceutically-acceptable salt thereof. In some embodiments, the present disclosure provides a method of treating hormone receptor positive, human epidermal growth factor receptor 2 (HER2) positive breast cancer in a subject in need thereof, the method comprising administering to the subject a therapeutically-effective amount of 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof. In some embodiments, the present disclosure provides a method of treating hormone receptor positive, human epidermal growth factor receptor 2 (HER2) positive breast cancer in a subject in need thereof, the method comprising administering to the subject a therapeutically-effective amount of 8-cyclopentyl-2-((4-(4-methylpiperazin-1-yl)phenyl)amino)-7-oxo-7,8-dihydropyrido[2,3-d]pyrimidine-6-carbonitrile monolactate.

In some embodiments, the breast cancer is an estrogen receptor positive, progesterone receptor positive, human epidermal growth factor receptor 2 (HER2) positive breast cancer. In some embodiments, the present disclosure provides a method of treating estrogen receptor positive, progesterone receptor positive, human epidermal growth factor receptor 2 (HER2) positive breast cancer in a subject in need thereof, the method comprising administering to the subject a therapeutically-effective amount of a compound of formula (I), or a pharmaceutically-acceptable salt thereof. In some embodiments, the present disclosure provides a method of treating estrogen receptor positive, progesterone receptor positive, human epidermal growth factor receptor 2 (HER2) positive breast cancer in a subject in need thereof, the method comprising administering to the subject a therapeutically-effective amount of 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof. In some embodiments, the present disclosure provides a method of treating estrogen receptor positive, progesterone receptor positive, human epidermal growth factor receptor 2 (HER2) positive breast cancer in a subject in need thereof, the method comprising administering to the subject a therapeutically-effective amount of 8-cyclopentyl-2-((4-(4-methylpiperazin-1-yl)phenyl)amino)-7-oxo-7,8-dihydropyrido[2,3-d]pyrimidine-6-carbonitrile monolactate.

In some embodiments, the breast cancer is an estrogen receptor negative, progesterone receptor negative, human epidermal growth factor receptor 2 (HER2) negative breast cancer. In some embodiments, the present disclosure provides a method of treating estrogen receptor negative, progesterone receptor negative, human epidermal growth factor receptor 2 (HER2) negative breast cancer in a subject in need thereof, the method comprising administering to the subject a therapeutically-effective amount of a compound of formula (I), or a pharmaceutically-acceptable salt thereof. In some embodiments, the present disclosure provides a method of treating estrogen receptor negative, progesterone receptor negative, human epidermal growth factor receptor 2 (HER2) negative breast cancer in a subject in need thereof, the method comprising administering to the subject a therapeutically-effective amount of 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof. In some embodiments, the present disclosure provides a method of treating estrogen receptor negative, progesterone receptor negative, human epidermal growth factor receptor 2 (HER2) negative breast cancer in a subject in need thereof, the method comprising administering to the subject a therapeutically-effective amount of 8-cyclopentyl-2-((4-(4-methylpiperazin-1-yl)phenyl)amino)-7-oxo-7,8-dihydropyrido[2,3-d]pyrimidine-6-carbonitrile monolactate.

In some embodiments, the breast cancer is a hormone receptor positive breast cancer. In some embodiments, the present disclosure provides a method of treating hormone receptor positive breast cancer in a subject in need thereof, the method comprising administering to the subject a therapeutically-effective amount of a compound of formula (I), or a pharmaceutically-acceptable salt thereof. In some embodiments, the present disclosure provides a method of treating hormone receptor positive breast cancer in a subject in need thereof, the method comprising administering to the subject a therapeutically-effective amount of 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof. In some embodiments, the present disclosure provides a method of treating hormone receptor positive breast cancer in a subject in need thereof, the method comprising administering to the subject a therapeutically-effective amount of 8-cyclopentyl-2-((4-(4-methylpiperazin-1-yl)phenyl)amino)-7-oxo-7,8-dihydropyrido[2,3-d]pyrimidine-6-carbonitrile monolactate.

In some embodiments, the breast cancer is an estrogen receptor positive breast cancer. In some embodiments, the present disclosure provides a method of treating estrogen receptor positive breast cancer in a subject in need thereof, the method comprising administering to the subject a therapeutically-effective amount of a compound of formula (I), or a pharmaceutically-acceptable salt thereof. In some embodiments, the present disclosure provides a method of treating estrogen receptor positive breast cancer in a subject in need thereof, the method comprising administering to the subject a therapeutically-effective amount of 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof. In some embodiments, the present disclosure provides a method of treating estrogen receptor positive breast cancer in a subject in need thereof, the method comprising administering to the subject a therapeutically-effective amount of 8-cyclopentyl-2-((4-(4-methylpiperazin-1-yl)phenyl)amino)-7-oxo-7,8-dihydropyrido[2,3-d]pyrimidine-6-carbonitrile monolactate.

In some embodiments, the breast cancer is a progesterone receptor positive breast cancer. In some embodiments, the present disclosure provides a method of treating progesterone receptor positive breast cancer in a subject in need thereof, the method comprising administering to the subject a therapeutically-effective amount of a compound of formula (I), or a pharmaceutically-acceptable salt thereof. In some embodiments, the present disclosure provides a method of treating progesterone receptor positive breast cancer in a subject in need thereof, the method comprising administering to the subject a therapeutically-effective amount of 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof. In some embodiments, the present disclosure provides a method of treating progesterone receptor positive breast cancer in a subject in need thereof, the method comprising administering to the subject a therapeutically-effective amount of 8-cyclopentyl-2-((4-(4-methylpiperazin-1-yl)phenyl)amino)-7-oxo-7,8-dihydropyrido[2,3-d]pyrimidine-6-carbonitrile monolactate.

In some embodiments, the breast cancer is a hormone receptor negative, human epidermal growth factor receptor 2 (HER2) positive breast cancer. In some embodiments, the present disclosure provides a method of treating hormone receptor negative, human epidermal growth factor receptor 2 (HER2) positive breast cancer in a subject in need thereof, the method comprising administering to the subject a therapeutically-effective amount of a compound of formula (I), or a pharmaceutically-acceptable salt thereof. In some embodiments, the present disclosure provides a method of treating hormone receptor negative, human epidermal growth factor receptor 2 (HER2) positive breast cancer in a subject in need thereof, the method comprising administering to the subject a therapeutically-effective amount of 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof. In some embodiments, the present disclosure provides a method of treating hormone receptor negative, human epidermal growth factor receptor 2 (HER2) positive breast cancer in a subject in need thereof, the method comprising administering to the subject a therapeutically-effective amount of 8-cyclopentyl-2-((4-(4-methylpiperazin-1-yl)phenyl)amino)-7-oxo-7,8-dihydropyrido[2,3-d]pyrimidine-6-carbonitrile monolactate.

In some embodiments, the breast cancer is a triple positive breast cancer. In some embodiments, a triple positive breast cancer is positive for an estrogen receptor overexpression, e.g., the tumor overexpresses an estrogen receptor, or expresses a higher amount of an estrogen receptor. In some embodiments, a triple positive breast cancer is positive for a progesterone receptor, e.g., the tumor overexpresses a progesterone receptor, or expresses a higher amount of a progesterone receptor. In some embodiments, a triple positive breast cancer is positive for human epidermal growth factor receptor 2 (HER2) protein, e.g., the tumor overexpresses human epidermal growth factor receptor 2 (HER2) protein or expresses a higher amount of human epidermal growth factor receptor 2 (HER2) protein. In some embodiments, a triple positive breast cancer overexpresses an estrogen receptor, a progesterone receptor, and a human epidermal growth factor receptor 2 (HER2) protein.

In some embodiments, the breast cancer is a triple negative breast cancer. In some embodiments, a triple negative breast cancer is negative for an estrogen receptor, e.g., the tumor does not express an estrogen receptor, or expresses a reduced amount of an estrogen receptor. In some embodiments, a triple negative breast cancer is negative for a progesterone receptor, e.g., the tumor does not express a progesterone receptor, or expresses a reduced amount of a progesterone receptor. In some embodiments, a triple negative breast cancer is negative for human epidermal growth factor receptor 2 (HER2) protein, e.g., the tumor does not express human epidermal growth factor receptor 2 (HER2) protein, or expresses a reduced amount of human epidermal growth factor receptor 2 (HER2) protein. In some embodiments, a triple negative breast cancer does not express any of an estrogen receptor, a progesterone receptor, or a human epidermal growth factor receptor 2 (HER2) protein.

In some embodiments, a triple negative breast cancer can express a BUB1 (Budding uninhibited by benzimidazoles 1), a mitotic checkpoint serine/threonine kinase. In some embodiments, a BUB1 gene can be upregulated in triple negative breast cancer.

In some embodiments, a breast cancer treated herein is an estrogen receptor negative, progesterone receptor negative, human epidermal growth factor receptor 2 (HER2) positive breast cancer. In some embodiments, a breast cancer treated herein is an estrogen receptor negative, progesterone receptor positive, human epidermal growth factor receptor 2 (HER2) positive breast cancer. In some embodiments, a breast cancer treated herein is an estrogen receptor positive, progesterone receptor negative, human epidermal growth factor receptor 2 (HER2) positive breast cancer. In some embodiments, a breast cancer treated herein is an estrogen receptor positive, progesterone receptor positive, human epidermal growth factor receptor 2 (HER2) positive breast cancer. In some embodiments, a breast cancer treated herein is an estrogen receptor negative, progesterone receptor negative, human epidermal growth factor receptor 2 (HER2) negative breast cancer. In some embodiments, the present disclosure provides a method of treating estrogen receptor negative, progesterone receptor negative, human epidermal growth factor receptor 2 (HER2) positive breast cancer in a subject in need thereof, the method comprising administering to the subject a therapeutically-effective amount of a compound of formula (I), or a pharmaceutically-acceptable salt thereof. In some embodiments, the present disclosure provides a method of treating estrogen receptor negative, progesterone receptor negative, human epidermal growth factor receptor 2 (HER2) positive breast cancer in a subject in need thereof, the method comprising administering to the subject a therapeutically-effective amount of 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof. In some embodiments, the present disclosure provides a method of treating estrogen receptor negative, progesterone receptor negative, human epidermal growth factor receptor 2 (HER2) negative breast cancer in a subject in need thereof, the method comprising administering to the subject a therapeutically-effective amount of 8-cyclopentyl-2-((4-(4-methylpiperazin-1-yl)phenyl)amino)-7-oxo-7,8-dihydropyrido[2,3-d]pyrimidine-6-carbonitrile monolactate.

In some embodiment, the administering comprises a second line of therapy. In some embodiments, the subject received a therapy other than the compound for the breast cancer (e.g., the hormone-receptor positive breast cancer) prior to the administering. In some embodiments, the therapy was received after the subject was diagnosed with the breast cancer. In some embodiments, the therapy was received after the subject was diagnosed with the hormone receptor positive breast cancer. In some embodiments, the therapy was received after the subject was diagnosed with the estrogen receptor positive breast cancer. In some embodiments, the subject did not respond to the therapy. In some embodiments, the subject experienced a relapse of the breast cancer after the therapy. In some embodiments, the subject experienced a relapse of the hormone receptor positive breast cancer after the therapy. In some embodiments, the subject experienced a relapse of the estrogen receptor positive breast cancer after the therapy. In some embodiments, the compounds, e.g., Compound 1 or a pharmaceutically acceptable salt thereof disclosed herein may induce irreversible cell proliferation inhibition. In some embodiments, the compounds, e.g., Compound 1 or a pharmaceutically acceptable salt thereof disclosed herein may inhibit autophagy, e.g., at an early stage, at a late stage. In some embodiments, the compounds, e.g., Compound 1 or a pharmaceutically acceptable salt thereof disclosed herein may engage more potential target compared to a comparator molecule. In some embodiments, Compound 1 treatment may promote anti-tumor immunity, e.g., by influencing the expression of various immune modulators in the tumor cells.

### Combination Therapy

The present disclosure also provides method for using such a compound disclosed herein, for example, 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof with one or more additional therapeutic agents. Methods disclosed herein further include administering one or more additional agents to treat a disease or disorder in a combination therapy. For example, in some embodiments, a combination therapy comprises administering a compound disclosed herein, for example, 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof, with (concurrently, simultaneously, or sequentially) a second agent. The compounds described herein for example, 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof, can be used in combination with agents disclosed herein or other suitable agents, depending on the condition being treated. In some embodiments the one or more compounds of the disclosure are co-administered with a second agent. When used in combination therapy, the compounds described herein can be administered with the second agent simultaneously or separately. This administration in combination can include simultaneous administration of the two agents in the same dosage form, simultaneous administration in separate dosage forms, and separate administration. A compound described herein, and a second agent can be formulated together in the same dosage form and administered simultaneously. In some embodiments, a compound disclosed herein and a second agent can be simultaneously administered, wherein both the agents are present in separate formulations. In some embodiments, a compound of the present disclosure can be administered just followed by a second agent, or vice versa. In some embodiments, a compound disclosed herein and a second agent are administered a one or more minutes apart, one or more hours apart, or one or more days apart.

In some embodiments, the second agent is a biological, pharmaceutical, or chemical compound. Non-limiting examples of a second agent include a simple or complex organic or inorganic molecule, a peptide, a protein, an oligonucleotide, an epigenetic modulator, hormones (steroidal or peptide), fusion molecules, an antibody, an antibody derivative, antibody fragment, a vitamin derivative, a carbohydrate, a toxin, a vaccine, e.g., cancer vaccine, a chemotherapeutic compound, radiotherapies (γ-rays, X-rays, and/or the directed delivery of radioisotopes, microwaves, and UV radiation), gene therapies (e.g., antisense, retroviral therapy) and other immunotherapies. Additional non-limiting examples of a second agent include small molecule inhibitors, monoclonal antibodies (mAbs), sdAbs, chimeric antigen receptors (CARs), CAR T-cell therapy, and antibody-drug conjugates (ADCs), and bispecific antibodies. In some embodiments, a second agent is a biologic. Non-limiting examples of biologics include vaccines, blood, and blood components, allergenics, somatic cells, gene therapy, tissues, and recombinant therapeutic proteins. In some embodiments, the method includes administering a procedure. Non-limiting examples of procedures include surgery, radiation treatments (i.e., beam radiation), chemotherapy, immunotherapy, and ablation.

In some embodiments, a combination therapy includes the combination of one or more compounds of the disclosure with a second agent to provide a synergistic or additive therapeutic effect.

In some embodiments, the second therapeutic agent is an autophagy initiating inhibitor, e.g., a small molecule kinase inhibitor, e.g., ULK1/2 inhibitor, e.g., SBI-0206965 (SBI). In some embodiments, the second therapeutic agent is anAOK5 inhibitor. In some embodiments, the second therapeutic agent is an autophagy inhibitor, e.g., chloroquine, hydroxychloroquine. In some embodiments, the second therapeutic agent is an aromatase inhibitor. In some embodiments, the aromatase inhibitor is letrozole or a pharmaceutically-acceptable salt thereof. In some embodiments, the second therapeutic agent is a selective estrogen receptor degrader. In some embodiments, the second therapeutic agent is a selective estrogen receptor blocker. In some embodiments, the selective estrogen receptor degrader is fulvestrant. In some embodiments, the second therapeutic agent is an autophagy agent, e.g., hydroxychloroquine, chloroquine.

In some embodiments, a compound of the disclosure, e.g., a compound of formula (I), such as 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof, is administered in combination with a second therapeutic agent. In some embodiments, a compound of the disclosure, e.g., a compound of formula (I), such as 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof, is administered in combination with an estrogen receptor modulator. In some embodiments, a compound of the disclosure, e.g., a compound of formula (I), such as 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof, is administered in combination with an estrogen receptor blocker, e.g., an aromatase inhibitor such as letrozole or a pharmaceutically-acceptable salt thereof. In some embodiments, a compound of the disclosure, e.g., a compound of formula (I), such as 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof, is administered in combination with a progestin such as megestrol or esters thereof (e.g., megestrol acetate). In some embodiments, a compound of the disclosure, e.g., a compound of formula (I), such as 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof, is administered in combination with an estrogen receptor degrader such as fulvestrant. In some embodiments, a compound of the disclosure, e.g., a compound of formula (I), such as 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof, is administered in combination with an angiogenesis inhibitor. In some embodiments, a compound of the disclosure, e.g., a compound of formula (I), such as 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof, is administered in combination with a vascular endothelial growth factor (VEGF) inhibitor. In some embodiments, a compound of the disclosure, e.g., a compound of formula (I), such as 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof, is administered in combination with a phosphoinositide 3-kinase (PI3K) inhibitor. In some embodiments, a combination therapy includes administration of two, three, four, or five additional agents in combination with a compound disclosed herein. In some embodiments, the third therapeutic agent can be a therapeutic agent disclosed herein. In some embodiments, the fourth therapeutic agent can be a therapeutic agent disclosed herein. In some embodiments, the fifth therapeutic agent can be a therapeutic agent disclosed herein.

### Estrogen Receptor Blockers

Estrogens stimulate or maintain the growth of some cancers (e.g., breast cancer). Treatment of breast cancer thought to be hormonally responsive (e.g., estrogen and/or progesterone receptor positive) is aimed at decreasing estrogen levels or inhibiting estrogen effects. In some embodiments, these interventions lead to decreased tumor mass or delayed progression of tumor growth.

Estrogen blockers block the production of estrogens (e.g., estradiol) or prevent estrogens (e.g., estradiol) from mediating biological effects in the body. Estrogen blockers act by blocking the estrogen receptor and/or directly inhibiting or suppressing estrogen production. Estrogen blockers are divided into classes by whether the estrogen blockers reduce the production of estrogen (aromatase inhibitors and antigonadotropins) or whether the estrogen blockers reduce the response to estrogen (antiestrogens and estrogen antagonists). By producing less estrogen, or by blocking the response to estrogen, estrogen blockers slow or inhibit the growth of cancer cells that require estrogen to stimulate growth (e.g., breast cells, uterine cells). Non-limiting examples of estrogen blockers include aromatase inhibitors, e.g., letrozole (Femara), anastrozole (Arimidex), exemestane (Aromasin), tamoxifen, testolactone (Teslac), ethamoxytriphetol, clomifene, and raloxifene.

Non-limiting examples of cells whose activity can be modulated by a combination therapy of a compound described herein and an estrogen blocker include secretory cells, cells with cilia, basal cells, red blood cells, mesenchymal cells, pluripotential mesenchymal cells, predecidual cells, epithelial cells, histiocytes, granulocytes, glandular cells, stromal cells, normal endometrial cells (NEMCs), atypical endometrial cells (AEMCs), and endometrial carcinoma cells (EMCCs).

Non-limiting examples of tumors that are treatable by a combination of a compound described herein and an estrogen blocker include solid tumors, solid tumors that are refractory to prior treatment with conventional chemotherapy, and solid tumors that respond to initial chemotherapy and subsequently relapse, as evidenced, e.g., by disease progression.

### Aromatase Inhibitors

In postmenopausal women, estrogens are mainly derived from the action of the aromatase enzyme. This enzyme converts adrenal androgens (e.g., androstenedione and testosterone) to estrone and estradiol. The suppression of estrogen biosynthesis in peripheral tissues and in the cancer tissue is achieved by inhibiting the aromatase enzyme.

Letrozole is a nonsteroidal competitive inhibitor of the aromatase enzyme system. Letrozole inhibits the conversion of androgens to estrogens. Letrozole selectively inhibits gonadal steroidogenesis but has no significant effect on adrenal mineralocorticoid or glucocorticoid synthesis. Letrozole inhibits the aromatase enzyme by competitively binding to the heme of the cytochrome P450 subunit of the enzyme, resulting in a reduction of estrogen biosynthesis. Treatment of women with letrozole significantly lowers serum estrone, estradiol and estrone sulfate.

The structure of letrozole is depicted below.

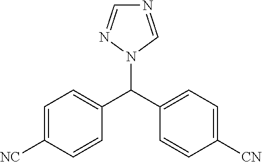 [SMILES: N#Cc1ccc(C(c2ccc(C#N)cc2)n2cncn2)cc1]

4,4′-((1H-1,2,4-triazol-1-yl)methylene)dibenzonitrile. A pharmaceutically-acceptable salt of 4,4′-((1H-1,2,4-triazol-1-yl)methylene)dibenzonitrile can also be used.

### Progestins

Progestins are synthetic forms of the naturally-occurring hormone progesterone. Progestins counteract estrogen effects on the body and function similarly to estrogen blockers. Non-limiting examples of progestins include megestrol acetate, acetomepregenol, chlormadinone acetate, cyproterone acetate, danazol, drospirenone, gestrinone, levonorgestrel, medrogestone, norethisterone, norethisterone acetate, norgestrel, oxendolone, osaterone acetate, trimegestone. norethindrone, norethindrone acetate, norethynodrel, ethynodiol diacetate, a third desogestrel, gestodene, norgestimate, dienogest, nestorone, and nomegestrol acetate.

Megestrol acetate is a synthetic derivative of progesterone. Megestrol binds to progesterone receptors and changes the hormone balance in the body. In some embodiments, this change can inhibit or stop cancers associated with estrogen.

The structure of megestrol acetate is depicted below.

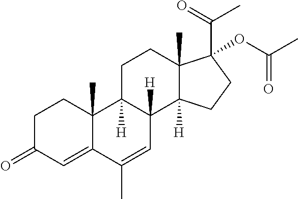

17-Hydroxy-6-methyl pregna-4,6-diene-3,20-dione acetate.

### Selective Estrogen Receptors Degraders

A selective estrogen receptor degrader or down-regulator (SERD) can bind to the estrogen receptor and cause the estrogen receptor to be degraded and thus downregulated. SERDs are used to treat estrogen receptor-sensitive or progesterone receptor-sensitive cancers along with other similar classes of drugs such as estrogen blockers and aromatase inhibitors.

Non-limiting examples of selective estrogen receptor degraders include rulvestrant, Giredestrant, Amcenestrant (SAR439859), AZD9833, Rintodestrant, LSZ102, LY3484356, ZN-c5, D-0502, and SHR9549.

In some embodiments, the selective estrogen receptor degrader is fulvestrant. Fulvestrant binds to and destabilizes the estrogen receptor, causing the cell's protein degradation processes to destroy the estrogen receptor.

The structure of fulvestrant is depicted below.

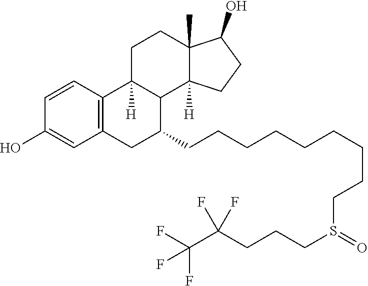

7α-[9-[(4,4,5,5,5-Pentafluoropentyl)-sulfinyl]nonyl]estra-1,3,5(10)-triene-3,17β-diol.

### Angiogenesis Inhibitors and Vascular Endothelial Growth Factor Inhibitors

Angiogenesis inhibitors interfere with blood vessel formation. Normally, the angiogenesis stimulating and inhibiting effects of these chemical signals are balanced so that blood vessels form only when and where vessels are needed. However, these signals can become unbalanced, causing increased blood vessel growth that can lead to abnormal conditions or disease, such as cancer.

Vascular endothelial growth factors (VEGF) are angiogenic factors that are growth factors for vascular endothelial cells. When VEGF and other endothelial growth factors bind to receptors on endothelial cells, signals within these cells are initiated that promote the growth and survival of new blood vessels.

Angiogenesis inhibitors interfere with various steps in blood vessel growth. Some angiogenesis inhibitors are monoclonal antibodies that specifically recognize and bind to VEGF to block the binding of VEGF to the VEGF receptor. Other angiogenesis inhibitors bind to VEGF, the VEGFs receptor, other receptors on the surface of endothelial cells, or to other proteins in the downstream signaling pathways. Some angiogenesis inhibitors are immunomodulatory drugs—agents that stimulate or suppress the immune system—that also have antiangiogenic properties. Non-limiting examples of VEGF inhibitors include bevacizumab (Avastin), sorafenib (Nexavar), sunitinib (Sutent), nilotinib (Tasigna), pazopanib (Votrient), and dasatinib (Sprycel).

### ULK1/2 Inhibitors

Unc-51-like kinase 1/2 (ULK 1/2) inhibitors inhibit the phosphorylation of ULK1 or ULK2, which regulates autophagy and lysosomal fusion, thereby blocking autophagy flux. ULK plays critical role during initial stages of autophagy which is a vital response to nutrient starvation. ULK1/2 is an essential and early autophagy regulator that are frequently activated in many cancers, e.g., KRAS mutant cancers. Non-limiting examples of ULK 1/2 include SBI-0206965 (SBI), MRT68921, DCC-3116, MRT67307, or pharmaceutical salts thereof.

### PI3K Inhibitors

Phosphoinositide 3-kinase (PI3K) inhibitors inhibit one or more of the phosphoinositide 3-kinase enzymes. These enzymes form part of the PI3K/AKT/mTOR pathway, which is a pathway involved in cell growth and survival, and other processes that are frequently activated in many cancers. By inhibiting these enzymes, PI3K inhibitors cause cell death, inhibit the proliferation of malignant cells, and interfere with several signaling pathways. PI3K inhibitors are usually given to treat certain cancers that have relapsed or are unresponsive to other cancer treatments. Non-limiting examples of PI3K inhibitors include alpelisib, copanlisib, duvelisib, and idelalisib.

### Autophagy Inhibitors

Small molecule inhibitors of autophagy can suppress tumor growth both in vitro and in vivo. Inhibition of autophagy sensitizes cancer cells to therapy, enhancing the cytotoxic effects induced by chemotherapeutic agents. Autophagy is a key pathway in the development of endocrine resistance in breast cancer. In some embodiments, targeting autophagy can reverse antiestrogen resistance. Autophagy inhibitors can be weak bases. Non-limiting examples of autophagy inhibitors include hydroxychloroquine (HCQ), chloroquine (CQ). In some embodiments, the unprotonated form of CQ/HCQ can diffuse through cell membranes and enter into organelles such as lysosomes, where the high concentration of H+ induces their protonation and consequently increases lysosomal pH.

### AMPK-Related Protein Kinase 5 Inhibitors

AMPK-related protein kinase 5 regulates Atk-dependent cell survival and migration (e.g., formation of metastases) through inhibition of cellular metabolism. ARK5 overexpression is found in multiple tumors and is associated with poor prognosis in metastatic breast cancer, multiple myeloma, and hepatocellular carcinoma. In some embodiments, inhibition of ARK5 induces cell death through PI3K/AKT/mTOR pathway.

### Administration of Compounds of the Disclosure

### Compound 1

A compound disclosed herein, for example, 8-cyclopentyl-2-((4-(4-methylpiperazin-1-yl)phenyl)amino)-7-oxo-7,8-dihydropyrido[2,3-d]pyrimidine-6-carbonitrile (formula 1), can be formulated as a capsule. A capsule can be a hard capsule. A capsule can be a soft capsule. A capsule can be a soft gelatin capsule. In some embodiments, a compound disclosed herein can be formulated as a hard capsule, the hard capsule comprising an amount of 8-cyclopentyl-2-((4-(4-methylpiperazin-1-yl)phenyl)amino)-7-oxo-7,8-dihydropyrido[2,3-d]pyrimidine-6-carbonitrile monolactate equivalent to 40 mg of 8-cyclopentyl-2-((4-(4-methylpiperazin-1-yl)phenyl)amino)-7-oxo-7,8-dihydropyrido[2,3-d]pyrimidine-6-carbonitrile. Alternatively, a compound, e.g., Compound 1 described herein can be formulated as a tablet.

In some embodiments, the present disclosure provides a pharmaceutical composition comprising, in a unit dosage form, an amount of 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof, equivalent to 40 mg of a compound described herein.

In some embodiments, the present disclosure provides a pharmaceutical composition comprising, in a unit dosage form, an amount of 8-cyclopentyl-2-((4-(4-methylpiperazin-1-yl)phenyl)amino)-7-oxo-7,8-dihydropyrido[2,3-d]pyrimidine-6-carbonitrile monolactate, equivalent to 40 mg of 8-cyclopentyl-2-((4-(4-methylpiperazin-1-yl)phenyl)amino)-7-oxo-7,8-dihydropyrido[2,3-d]pyrimidine-6-carbonitrile.

In some embodiments, the present disclosure provides a pharmaceutical composition comprising, in a unit dosage form, 48.4 mg of 8-cyclopentyl-2-((4-(4-methylpiperazin-1-yl)phenyl)amino)-7-oxo-7,8-dihydropyrido[2,3-d]pyrimidine-6-carbonitrile monolactate.

In some embodiments, 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt is administered in oral capsules, swallowed with water in the morning in a fasted state, at least 1 hour before ingesting food. In some embodiments, a morning dose is taken after an overnight fast an hour before ingesting food. In some embodiments, a compound described herein is administered every day. In some embodiments, a compound described herein is administered every day for 4 weeks. In some embodiments, a compound described herein is on a 4-week cycle of: (i) a continuous, three-week period of once-daily administration; and (ii) immediately following the three-week period, one week of no administration. In some embodiments, a compound described herein is administered every 2 days.

### Letrozole

A compound disclosed herein, for example, letrozole, can be formulated as tablet. In some embodiments, letrozole can be formulated as a tablet, the tablet comprising, in a unit dosage form, a therapeutically-effective amount of letrozole, and a pharmaceutically-acceptable excipient.

In some embodiments, letrozole is provided as 2.5 mg tablets for oral administration. The tablets can be colored, e.g., yellow, and can be uncoated or film-coated. Inactive ingredients can include colloidal silicon dioxide, ferric oxide, hydroxypropyl methylcellulose, lactose monohydrate, magnesium stearate, maize starch, microcrystalline cellulose, polyethylene glycol, sodium starch glycolate, talc, and titanium dioxide. In some embodiments, letrozole is given at a dose of one 2.5 mg tablet administered once a day without regards to food.

### Fulvestrant

A compound disclosed herein, for example, fulvestrant, can be formulated as a solution, for example, a solution for intramuscular injection. In some embodiments, fulvestrant can be supplied as a 50 mg/mL solution comprising fulvestrant and pharmaceutically-acceptable excipients and/or co-solvents. In some embodiments, fulvestrant can be supplied as a 50 mg/mL solution comprising 10% w/v alcohol, 10% w/v benzyl alcohol, and 15% w/v benzyl benzoate as co-solvents, and made up to 100% w/v castor oil as a co-solvent and release-rate modifier.

In some embodiments, a fulvestrant dose of 500 mg can be administered intramuscularly. In some embodiments, a fulvestrant dose of 250 mg can be administered intramuscularly. In some embodiments, fulvestrant can be administered intramuscularly into the gluteal area slowly as two 5 mL injections (one injection per buttock, 1-2 minutes per injection) on days 1, 15, 29, and once monthly thereafter.

### Megestrol Acetate

A compound disclosed herein, for example, megestrol acetate, can be formulated as a suspension, for example a suspension suitable for oral administration. In some embodiments, megestrol acetate oral suspension contains 625 mg of megestrol acetate per 5 mL (125 mg/mL). In some embodiments, megestrol acetate oral suspension contains 800 mg per 20 mL (40 mg/mL). In some embodiments, megestrol acetate oral suspension contains one or more of the following inactive ingredients: alcohol (up to 0.06% v/v), lime flavor, citric acid monohydrate, docusate sodium, hydroxypropyl methylcellulose, natural and artificial lemon flavor, purified water, sodium benzoate, sodium citrate, dihydrate, and sucrose.

In some embodiments, megestrol acetate can be formulated as a tablet. In some embodiments, tablets contain 20 mg megestrol acetate. In some embodiments, tablets contain 40 mg megestrol acetate. In some embodiments, megestrol acetate tablets contain one or more of the following inactive ingredients: acacia spray dried, colloidal silicon dioxide, corn starch, di-calcium phosphate dihydrate powder, lactose hydrous impalpable, magnesium stearate, and pregelatinized starch.

### Pharmaceutical Compositions

A pharmaceutical composition of the disclosure can be a combination of any pharmaceutical compounds described herein with other chemical components, such as carriers, stabilizers, diluents, dispersing agents, suspending agents, thickening agents, and/or excipients. A pharmaceutical composition can comprise Compound 1 or Compound 1 salt and a pharmaceutically acceptable excipient. The pharmaceutical composition facilitates administration of the compound to an organism. Pharmaceutical compositions can be administered in therapeutically-effective amounts as pharmaceutical compositions by various forms and routes including, for example, intravenous, subcutaneous, intramuscular, inhalation, oral, parenteral, ophthalmic, optic, subcutaneous, transdermal, nasal, intravitreal, intratracheal, intrapulmonary, transmucosal, vaginal, and topical administration.

Formulations can be modified depending upon the route of administration chosen. Pharmaceutical compositions comprising a compound described herein can be manufactured, for example, by mixing, dissolving, emulsifying, encapsulating, entrapping, or compression processes.

Non-limiting examples of dosage forms suitable for use in a method disclosed herein include feed, food, pellet, lozenge, liquid, elixir, aerosol, inhalant, spray, powder, tablet, pill, capsule, gel, geltab, nanosuspension, nanoparticle, microgel, suppository troches, aqueous or oily suspensions, ointment, patch, lotion, dentifrice, emulsion, creams, drops, dispersible powders or granules, emulsion in hard or soft gel capsules, syrups, phytoceuticals, nutraceuticals, and any combination thereof.

Pharmaceutical compositions can be formulated by combining the active compounds with pharmaceutically-acceptable carriers or excipients. Non-limiting examples of pharmaceutically-acceptable excipients suitable for use in the method disclosed herein include granulating agents, binding agents, lubricating agents, disintegrating agents, sweetening agents, glidants, anti-adherents, anti-static agents, surfactants, anti-oxidants, gums, coating agents, coloring agents, flavoring agents, coating agents, plasticizers, preservatives, suspending agents, emulsifying agents, anti-microbial agents, plant cellulosic material and spheronization agents, and any combination thereof.

Non-limiting examples of pharmaceutically-acceptable carriers include saline solution, Ringer's solution and dextrose solution. Further carriers include sustained release preparations such as semipermeable matrices of solid hydrophobic polymers containing the compound disclosed herein, where the matrices are in the form of shaped articles, such as films, liposomes, microparticles, and microcapsules.

For oral administration, pharmaceutical compositions can be formulated by combining the active compounds with pharmaceutically-acceptable carriers or excipients into a unit dosage form which can be solid or liquid. Non-limiting examples of oral solid forms include tablets, powders, pills, dragees, capsules, liquids, gels, syrups, elixirs, slurries, or suspensions for oral ingestion by a subject. Pharmaceutical preparations for oral use can be obtained by mixing one or more solid excipients with one or more compounds described herein, optionally grinding the resulting mixture, and processing the mixture of granules, after adding suitable auxiliaries, if desired, to obtain tablets or dragee cores. Cores can be provided with suitable coatings. For this purpose, concentrated sugar solutions can be used. The solutions can contain an excipient such as gum arabic, talc, polyvinylpyrrolidone, carbopol gel, polyethylene glycol, or titanium dioxide, lacquer solutions, and suitable organic solvents or solvent mixtures. Dyestuffs or pigments can be added to the tablets or dragee coatings, for example, for identification or to characterize different combinations of active compound doses.

Pharmaceutical preparations that can be used orally include push-fit capsules made of gelatin and soft, sealed capsules made of gelatin and a plasticizer, such as glycerol or sorbitol. Pharmaceutical preparations that can be used orally include coated and uncoated tablets. In some embodiments, the capsule comprises a hard gelatin capsule, the capsule comprising one or more of pharmaceutical, bovine, and plant gelatins. A gelatin can be alkaline-processed. The capsule or tablet can contain the active ingredients in admixture with filler such as lactose, binders such as starches, or lubricants such as talc or magnesium stearate, and stabilizers. In soft capsules, the active compounds can be dissolved or suspended in suitable liquids, such as fatty oils, liquid paraffin, or liquid polyethylene glycols. Stabilizers can be added. All formulations for oral administration are provided in dosages suitable for such administration.

For oral administration of a liquid unit dosage form, pharmaceutical compositions can be formulated by combining the active compounds with pharmaceutically-acceptable carriers or excipients. Such carriers can be used to formulate liquids, gels, syrups, elixirs, slurries, or suspensions, for oral ingestion by a subject. Non-limiting examples of solvents used in an oral dissolvable formulation can include water, ethanol, isopropanol, saline, physiological saline, DMSO, dimethylformamide, potassium phosphate buffer, phosphate buffer saline (PBS), sodium phosphate buffer, 4-2-hydroxyethyl-1-piperazineethanesulfonic acid buffer (HEPES), 3-(N-morpholino)propanesulfonic acid buffer (MOPS), piperazine-N,N′-bis(2-ethanesulfonic acid) buffer (PIPES), and saline sodium citrate buffer (SSC). Non-limiting examples of co-solvents used in an oral dissolvable formulation can include sucrose, urea, cremaphor, DMSO, and potassium phosphate buffer.

Parenteral injections can be formulated for bolus injection or continuous infusion. The pharmaceutical compositions can be in a form suitable for parenteral injection as a sterile suspension, solution or emulsion in oily or aqueous vehicles such as saline or water for injection, and can contain formulatory agents such as suspending, stabilizing and/or dispersing agents. Pharmaceutical formulations for parenteral administration include aqueous solutions of the active compounds in water-soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. The suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

The active compounds can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives. The compounds of the disclosure can be applied topically to the skin, or a body cavity, for example, oral, vaginal, bladder, cranial, spinal, thoracic, or pelvic cavity of a subject. The compounds of the disclosure can be applied to an accessible body cavity.

The compounds can also be formulated in rectal compositions such as enemas, rectal gels, rectal foams, rectal aerosols, suppositories, jelly suppositories, or retention enemas, containing conventional suppository bases such as cocoa butter or other glycerides, and synthetic polymers such as polyvinylpyrrolidone and PEG. In suppository forms of the compositions, a low-melting wax such as a mixture of fatty acid glycerides, optionally in combination with cocoa butter, can be used.

For buccal or sublingual administration, the compositions can be tablets, lozenges, or gels.

Formulations suitable for transdermal administration of the active compounds can employ transdermal delivery devices and transdermal delivery patches, and can be lipophilic emulsions or buffered aqueous solutions, dissolved or dispersed in a polymer or an adhesive. Such patches can be constructed for continuous, pulsatile, or on demand delivery of pharmaceutical compounds. Transdermal delivery can be accomplished by iontophoretic patches. Transdermal patches can provide controlled delivery. The rate of absorption can be slowed by using rate-controlling membranes or by trapping the compound within a polymer matrix or gel. Absorption enhancers can be used to increase absorption. An absorption enhancer or carrier can include absorbable pharmaceutically-acceptable solvents to assist passage through the skin. For example, transdermal devices can be in the form of a bandage comprising a backing member, a reservoir containing compounds and carriers, a rate controlling barrier to deliver the compounds to the skin of the subject at a controlled and predetermined rate over a prolonged period of time, and adhesives to secure the device to the skin or the eye.

For administration by inhalation, the active compounds can be in a form as an aerosol, a vapor, a mist, or a powder. Inhalation can occur through by nasal delivery, oral delivery, or both.

Nasal or intranasal administration involves insufflation of compounds through the nose, for example, nasal drops and nasal sprays. This route of administration can result in local and/or systemic effects. Inhaler or insufflator devices can be used for nose-to-lung delivery of compounds described herein.

A pharmaceutical composition can be administered in a local or systemic manner, for example, via injection of the compound directly into an organ, optionally in a depot or sustained release formulation or implant. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

In practicing the methods of treatment or use provided herein, therapeutically-effective amounts of the compounds described herein are administered in pharmaceutical compositions to a subject having a disease or condition to be treated. In some embodiments, the subject is a mammal such as a human. A therapeutically-effective amount can vary widely depending on the severity of the disease, the age and relative health of the subject, the potency of the compounds used, and other factors. The compounds can be used singly or in combination with one or more therapeutic agents as components of mixtures.

Methods for the preparation of compositions comprising the compounds described herein include formulating the compounds with one or more inert, pharmaceutically-acceptable excipients or carriers to form a solid, semi-solid, or liquid composition. Solid compositions include, for example, powders, tablets, dispersible granules, capsules, and cachets. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions, and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in the disclosure include liquid, powder, gel, nanosuspension, nanoparticle, microgel, aqueous or oily suspensions, emulsion, and any combination thereof.

Non-limiting examples of pharmaceutically-acceptable excipients suitable for use in the disclosure include binding agents, disintegrating agents, anti-adherents, anti-static agents, surfactants, anti-oxidants, coating agents, coloring agents, plasticizers, preservatives, suspending agents, emulsifying agents, anti-microbial agents, spheronization agents, solubilizers, stabilizers, tonicity enhancing agents, buffers and any combination thereof.

A composition of the disclosure can be, for example, an immediate release form or a controlled release formulation. An immediate release formulation can be formulated to allow the compounds to act rapidly. Non-limiting examples of immediate release formulations include readily dissolvable formulations. A controlled release formulation can be a pharmaceutical formulation that has been adapted such that release rates and release profiles of the active agent can be matched to physiological and chronotherapeutic requirements or, alternatively, has been formulated to effect release of an active agent at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

In some, a controlled release formulation is a delayed release form. A delayed release form can be formulated to delay a compound's action for an extended period of time. A delayed release form can be formulated to delay the release of an effective dose of one or more compounds, for example, for about 4, about 8, about 12, about 16, or about 24 h.

A controlled release formulation can be a sustained release form. A sustained release form can be formulated to sustain, for example, the compound's action over an extended period of time. A sustained release form can be formulated to provide an effective dose of any compound described herein (e.g., provide a physiologically-effective blood profile) over about 4, about 8, about 12, about 16 or about 24 h.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

### Subjects/Patient Population

Subjects can be, humans for example, elderly adults, adults, adolescents, pre-adolescents, children, toddlers, infants, neonates, and non-human animals, e.g., a mouse. In some embodiments, a subject is a patient. In some embodiments, a subject has experienced and/or exhibited at least one symptom of the disease or disorder to be treated and/or reduced in likelihood to occur. In some embodiments, the subject has been identified or diagnosed as having a cancer described herein, e.g., breast cancer. In some embodiments, the subject has a cancer and/or tumor that is positive for BUB1 expression. In some embodiments, the subject has a cancer and/or tumor that is positive for BRCA1 expression. In some embodiments, the subject is predisposed and/or at risk to having a cancer, e.g., breast cancer, based on presence of a mutation in a gene, e.g., BRCA1 mutation. In some embodiments, the subject has received a first line of therapy. In some embodiments, the subject is resistant and/or non-responsive to the first line of therapy.

In some embodiments, a method of treatment disclosed herein comprises, identification of a patient population based on one or more selection criteria, e.g., biomarkers, failure to respond to a primary therapy and administering a compound disclosed herein, e.g., Compound 1 to treat the patient. The patient population selection criteria can include but are not limited to, presence of a biomarker, e.g., marker associate with a particular disease, a marker associated with poor prognosis of a disease, failure to respond to an initial therapy, age, gender, health of the patient. The screening procedure may include but are not limited to blood and/or tissue sample analysis, genetic tests, genetic screening, biopsy, drug sensitivity/resistance test.

A method disclosed herein relates to administering the compound disclosed herein as part of a pharmaceutical composition. In some embodiments, a pharmaceutical composition comprises a compound described herein and a pharmaceutically-acceptable excipient. In some embodiments, compositions of a compound disclosed herein can comprise a liquid comprising an active agent in solution, in suspension, or both. Liquid compositions can include gels. In some embodiments, the liquid composition is aqueous. In some embodiments, the composition is an ointment. In some embodiments, the composition is an in situ gellable aqueous composition. In some embodiments, the composition is an in situ gellable aqueous solution.

A pharmaceutically-acceptable excipient can be present in a pharmaceutical composition at a mass of between about 0.1% and about 99% by mass of the composition. For example, a pharmaceutically-acceptable excipient can be present in a pharmaceutical composition at a mass of between about 0.1% and about 95%, between about 0.1% and about 90%, between about 0.1% and about 85%, between about 0.1% and about 80%, between about 0.1% and about 75%, between about 0.1% and about 70%, between about 0.1% and about 65%, between about 0.1% and about 60%, between about 0.1% and about 55%, between about 0.1% and about 50%, between about 0.1% and about 45%, between about 0.1% and about 40%, between about 0.1% and about 35%, between about 0.1% and about 30%, between about 0.1% and about 25%, between about 0.1% and about 20%, between about 0.1% and about 15%, between about 0.1% and about 10%, between about 0.1% and about 5%, or between about 0.1% and about 1%, by mass of the formulation.

A pharmaceutically-acceptable excipient can be present at about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% by mass of the formulation.

### Dosing

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged injectables, vials, or ampoules. Aqueous suspension compositions can be packaged in single-dose non-reclosable containers. Multiple-dose reclosable containers can be used, for example, in combination with or without a preservative. Formulations for parenteral injection can be presented in unit dosage form, for example, in ampoules, or in multi-dose containers with a preservative.

A compound described herein can be present in a composition in a range of from about 1 mg to about 5 mg, from about 5 mg to about 10 mg, from about 10 mg to about 15 mg, from about 15 mg to about 20 mg, from about 20 mg to about 25 mg, from about 25 mg to about 30 mg, from about 30 mg to about 35 mg, from about 35 mg to about 40 mg, from about 40 mg to about 45 mg, from about 45 mg to about 50 mg, from about 50 mg to about 55 mg, from about 55 mg to about 60 mg, from about 60 mg to about 65 mg, from about 65 mg to about 70 mg, from about 70 mg to about 75 mg, from about 75 mg to about 80 mg, from about 80 mg to about 85 mg, from about 85 mg to about 90 mg, from about 90 mg to about 95 mg, from about 95 mg to about 100 mg, from about 100 mg to about 125 mg, from about 125 mg to about 150 mg, from about 150 mg to about 175 mg, from about 175 mg to about 200 mg, from about 200 mg to about 225 mg, from about 225 mg to about 250 mg, or from about 250 mg to about 300 mg.

A compound described herein can be present in a composition in an amount of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 225 mg, about 250 mg, or about 300 mg.

A compound described herein can be administered to a subject in an amount of about 0.1 mg/kg to about 500 mg/kg, about 1 mg/kg to about 500 mg/kg, about 0.1 mg/kg to about 300 mg/kg, about 1 mg/kg to about 300 mg/kg, or about 0.1 mg/kg to about 30 mg/kg. In some embodiments, the compound disclosed herein is administered to a subject in an amount of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 120 mg/kg, about 150 mg/kg, about 160 mg/kg, about 180 mg/kg, about 200 mg/kg, about 240 mg/kg, about 250 mg/kg, about 300 mg/kg, about 350 mg/kg, about 360 mg/kg, about 400 mg/kg, about 450 mg/kg, about 500 mg/kg, or about 600 mg/kg of the subject.

### Dosing Regimens

A dosing regimen disclosed herein can be, for example, once a day, twice a day, thrice a day, once a week, twice a week, or thrice a week. In some embodiments, a compound disclosed herein is administered once daily. In some embodiments, a compound disclosed herein is administered once daily for 28 days (one cycle). In some embodiments, a compound disclosed herein is administered once daily in one or more 28 day cycles. In some embodiments, a compound disclosed herein is administered in a four-week cycle of consecutive once daily administration for three weeks, followed by one week with no administrations.

In some embodiments, the present disclosure provides a method of treating breast cancer in a subject in need thereof, the method comprising administering to the subject a therapeutically-effective amount of a compound of formula (I)

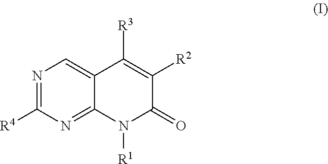

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or —CN, hydrogen, or halogen;
   R⁴ is alkyl, alkenyl, alkynyl, cycloalkyl, aryl, heterocyclyl, heteroaryl, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or —CN, hydrogen, or halogen;
provided that, when R⁴ is —NR⁵R⁶ and one of R⁵ and R⁶ is pyridyl, then R² is not —C(O)CH₃,
or a pharmaceutically-acceptable salt thereof;
wherein the administering is once daily for at least 4 weeks.

In some embodiments, the present disclosure provides a method of treating breast cancer in a subject in need thereof, the method comprising administering to the subject a therapeutically-effective amount of a compound of formula (I)

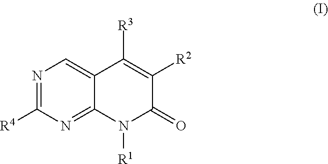

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or —CN, hydrogen, or halogen;
   R⁴ is alkyl, alkenyl, alkynyl, cycloalkyl, aryl, heterocyclyl, heteroaryl, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or —CN, hydrogen, or halogen;
provided that, when R⁴ is —NR⁵R⁶ and one of R⁵ and R⁶ is pyridyl, then R² is not —C(O)CH₃,
or a pharmaceutically-acceptable salt thereof;
   wherein the administering is a 4-week cycle of: (i) a continuous, three-week period of once-daily administration; and ii) immediately following the three-week period, one week of no administration.

A compound described herein can be administered before, during, or after the occurrence of a disease or condition, and the timing of administering the composition containing a compound can vary. For example, a compound can be used as a prophylactic and can be administered continuously to subjects with a propensity to conditions or diseases to lessen or reduce a likelihood of the occurrence of the disease or condition. A compound and composition can be administered to a subject during or as soon as possible after the onset of the symptoms. The administration of a compound can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein.

A compound can be administered as soon as is practical after the onset of a disease or condition is detected or suspected, and for a length of time necessary for the treatment of the disease, such as, for example, from about 1 month to about 3 months. In some embodiments, the length of time a compound can be administered can be about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months, about 22 months about 23 months, about 2 years, about 2.5 years, about 3 years, about 3.5 years, about 4 years, about 4.5 years, about 5 years, about 6 years, about 7 years, about 8 years, about 9 years, about 10 years, about 11 years, about 12 years, about 13 years, about 14 years, about 15 years, about 16 years, about 17 years, about 18 years, about 19 years, about 20 years, about 21 years, about 22 years, about 23 years, about 24 years, or about 25 years. The length of treatment can vary for each subject.

A dosing schedule for administration of a compound described herein can be consistent for the length of the dosing regimen. For example, a compound can be administered daily. Alternatively, or in addition to, a dosing schedule for administration of a compound described herein can include portions of time where dosing is paused. For example, a compound can be administered every day for 3 weeks and then not be administered for one week.

A dosing schedule for administration of a compound described herein can include once daily (QD), twice daily (BID), three times daily (TID), four times daily (QID), once weekly, twice weekly, three times weekly, once monthly, twice monthly, and once every other month. For example, a daily dose can be given in a single dose or divided into multiple doses to be administered in intervals, e.g., twice daily, three times daily, and the like. For example, a daily dose of 100 mg can be given, for example, once daily (100 mg), twice daily (50 mg per dose).

Multiple therapeutic agents can be administered in any order or simultaneously. In some embodiments, a compound of the disclosure is administered in combination with, before, or after treatment with another therapeutic agent, e.g., a drug, such as an aromatase inhibitor. In some embodiments, a compound of the disclosure is administered at regular intervals, such as, for example, once daily, twice daily, thrice daily, etc. and the second therapeutic agent is administered daily or intermittently or on an as-needed basis. If simultaneously, the multiple therapeutic agents can be provided in a single, unified form, or in multiple forms, for example, as multiple separate unit dosage forms. The agents can be packed together or separately, in a single package or in a plurality of packages. One or all the therapeutic agents can be given in multiple doses. If not simultaneous, the timing between the multiple doses can vary to as much as about a month.

### Compound 1 Dosing Regimens

A dosing regimen disclosed herein can be, for example, one dose of 40 mg, one dose of 80 mg, one dose of 120 mg; one dose of 160 mg, or one dose of 200 mg of oral 8-cyclopentyl-2-((4-(4-methylpiperazin-1-yl)phenyl)amino)-7-oxo-7,8-dihydropyrido[2,3-d]pyrimidine-6-carbonitrile per day. Alternatively, the dosing regimen disclosed herein can be, for example, 40 mg twice daily, 60 mg twice daily, 80 mg twice daily, or 100 mg twice daily. In some embodiments, the dosing is oral.

In some embodiments, 8-cyclopentyl-2-((4-(4-methylpiperazin-1-yl)phenyl)amino)-7-oxo-7,8-dihydropyrido[2,3-d]pyrimidine-6-carbonitrile is administered once daily. In some embodiments, 8-cyclopentyl-2-((4-(4-methylpiperazin-1-yl)phenyl)amino)-7-oxo-7,8-dihydropyrido[2,3-d]pyrimidine-6-carbonitrile is administered once daily for 28 days (one cycle). In some embodiments, 8-cyclopentyl-2-((4-(4-methylpiperazin-1-yl)phenyl)amino)-7-oxo-7,8-dihydropyrido[2,3-d]pyrimidine-6-carbonitrile is administered once daily in one or more 28 day cycles. In some embodiments, 8-cyclopentyl-2-((4-(4-methylpiperazin-1-yl)phenyl)amino)-7-oxo-7,8-dihydropyrido[2,3-d]pyrimidine-6-carbonitrile is administered in a four-week cycle of consecutive once daily administration for three weeks, followed by one week with no administrations.

In some embodiments, the present disclosure provides a method of treating estrogen receptor positive breast cancer in a subject in need thereof, the method comprising administering to the subject a therapeutically-effective amount of 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof, wherein the administering is once daily for at least 4 weeks.

In some embodiments, the present disclosure provides a method of treating estrogen receptor positive breast cancer in a subject in need thereof, the method comprising administering to the subject a therapeutically-effective amount of 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof, wherein the administering is a 4-week cycle of: (i) a continuous, three-week period of once-daily administration; and ii) immediately following the three-week period, one week of no administration.

In some embodiments, the present disclosure provides a method of treating estrogen receptor positive breast cancer in a subject in need thereof, the method comprising administering to the subject a therapeutically-effective amount of 8-cyclopentyl-2-((4-(4-methylpiperazin-1-yl)phenyl)amino)-7-oxo-7,8-dihydropyrido[2,3-d]pyrimidine-6-carbonitrile monolactate, wherein the administering is once daily for at least 4 weeks.

In some embodiments, the present disclosure provides a method of treating estrogen receptor positive breast cancer in a subject in need thereof, the method comprising administering to the subject a therapeutically-effective amount of 8-cyclopentyl-2-((4-(4-methylpiperazin-1-yl)phenyl)amino)-7-oxo-7,8-dihydropyrido[2,3-d]pyrimidine-6-carbonitrile monolactate, wherein the administering is a 4-week cycle of: (i) a continuous, three-week period of once-daily administration; and ii) immediately following the three-week period, one week of no administration.

### Letrozole Dosing Regimens

A dosing regimen disclosed herein can be, for example, once a day, twice a day, thrice a day, once a week, twice a week, or thrice a week. In some embodiment, the dosing is oral. In some embodiments, a suitable amount of letrozole can range from about 0.1 mg to about 100 mg per day, for example about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 2.5 mg, about 3 mg, about 3.5 mg, about 4 mg, about 4.5 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, or about 50 mg per day.

### Fulvestrant Dosing Regimens

A dosing regimen disclosed herein can be, for example, once a day, once a week, once every two weeks, once every three weeks, or once a month. In some embodiment, the dosing is intramuscular. In some embodiments, a suitable amount Fulvestrant dose can range from about 100 to about 1,000 mg per dose, for example about 100 mg, about 150 mg, about 200 mg, about 250, about 300, about 350 mg, about 400 mg, about 450 mg, about 500 mg, about 550 mg, about 600 mg, about 650 mg, about 700 mg, about 750 mg, about 800 mg, about 850 mg, about 900 mg, or about 1,000 mg per dose. In some embodiments Fulvestrant is administered intramuscularly. In some embodiments Fulvestrant is administered into the buttocks (gluteal area). In some embodiments Fulvestrant is administered into the buttocks (gluteal area) slowly (e.g., 1-2 minutes per injection) as two 5 mL injections, one in each buttock. In some embodiments, Fulvestrant is administered once every two weeks. In some embodiments, Fulvestrant is administered once a month. In some embodiments, Fulvestrant is administered on Days 1, 15, 29, and once monthly thereafter. In some embodiments, a composition of the disclosure binds to different cellular proteins than a comparator molecule. In some embodiments, a composition of the disclosure binds to different cellular proteins than palbociclib. In some embodiments, a composition of the disclosure binds to one or more of the following cellular proteins: CDC42BPB, CHEK1, DGCR6, MAP-KAPK5, TBK1, UVSSA, ZNF260, AAK1, ATAT1, AURKA, BMP2K, BUB1, CDK2, CDK5, CPQ, EPHA2, FKBP8, GSK3A, GSK3B, LIMK1, MAP11, PER3, SLK, STK17A, STK17B, TFEB, TGFBR2, USF2, ZCRB1, ACAA1, AIFM2, ANP32B, AP2A1, AP2M1, AP2S1, AVEN, BOD1L1, CDK12, CDK7, CDKN1A, CHEK1, COG8, DECR1, EIF4G1, FOS, FOSB, FOXK1, LYN, LYPLA2, MAP2K5, MAP3K20, MARK2, MICALL2, NPM1, PUS7L, RPS6KA3, SART3, SCP2, SHCBP1, SMAD1, SNX30, SNX6, STK3, TLE5, YES1, ZNF608, AAK1, ADAR, AGRN, AHCTF1, APLP2, APP, ARRB2, AXL, BMP2K, CAVINI, CAVIN3, CBX5, CCN1, CDC5L, CDK17, CELF1, CERS2, CLPP, CLUH, COL2A1, CPD, CRAT, CSNK2A2, CST3, DBNDD2, DDX21, DHX9, DNAJC13, ECE1, ELAVL1, ELL, ESYT1, FASTKD5, FBRS, FGFRL1, FUBP3, GAK, GALNT7, GCAT, GSK3A, GSK3B, GSKIP, HNRNPAO, HNRNPA1, HNRNPA2B1, HNRNPA3, HNRNPC, HNRNPL, HNRNPR, HNRNPU, HNRNPU, HNRNPUL2, HSPA13, HSPG2, IFT52, IGFBP4, IGFBP7, ILF3, INCENP, JMY, LAMA5, LAMC1, LAMTOR2, LEO1, LMAN2, LOXL2, LRCH4, LRPPRC, LRRC59, LTBP2, MAMDC2, MAN2A1, MATR3, METTL18, MLEC, MPHOSPH8, MRC2, MRPL55, MTG1, NAP1L1, NAPG, NRP1, PHF8, PIK3C₃, PIK3R⁴, PIP4K2A, PIP4K2B, PIP4P2, PRPF4B, PTX3, RAEl, RBM34, RIPK1, RNF5, RRAS2, RSF1, SCAF1, SGPP1, SIK2, SMU1, SND1, STK10, STX5, SYNCRIP, SYNE2, TAF15, TIMP2, TTK, UFL1, WTAP, and ZFR.

In some embodiments, a composition of the disclosure deregulates cellular phosphopeptide levels. In some embodiments, a composition of the disclosure deregulates a unique cellular phosphopeptide profile compared to a comparator molecule. In some embodiments, a composition of the disclosure deregulates one or more peptides selected from the list consisting of: PRKD1, ULK1, RAF1, MAP2K2, CAMK2D, LYN, PRKD2, AKT2, TLK1, GTF2F1, STK3, CAMK1, LATS1, BRAF, NUAK1, HCK, BUB1, ARAF, MAP2K5, CDK16, and MAPK7.

In some embodiments, a composition of the disclosure binds to BUB1 in a binding conformation different than comparator molecules. In some embodiments, a composition of the disclosure binds to BUB1 in a binding conformation different than palbociclib or abemaciclib. In some embodiments, a composition of the disclosure interacts with different BUB1 active site residues than comparator molecules. In some embodiments, a composition of the disclosure inter-acts with different BUB1 active site residues than palbociclib or abemacilib. In some embodiments, a composition of the disclosure hydrogen bonds with Y869 and D946 of BUB1, and has hydrophobic interactions with V819, K821, M850, G872, T873, N876, D921, 1924, and 1945 of BUB1.

In some embodiments, a composition of the disclosure decreases BUB1 protein levels. In some embodiments, a composition of the disclosure decreases BUB1 protein levels by at least 5%. In some embodiments, a composition of the disclosure decreases BUB1 protein levels by at least 10%. In some embodiments, a composition of the disclosure decreases BUB1 protein levels by at least 20%. In some embodiments, a composition of the disclosure decreases BUB1 protein levels by at least 30%. In some embodiments, a composition of the disclosure decreases BUB1 protein levels by at least 40%. In some embodiments, a composition of the disclosure decreases BUB1 protein levels by at least 50%. In some embodiments, a composition of the disclosure decreases BUB1 protein levels by at least 60%. In some embodiments, a composition of the disclosure decreases BUB1 protein levels by at least 70%. In some embodiments, a composition of the disclosure decreases BUB1 protein levels by at least 80%. In some embodiments, a composition of the disclosure decreases BUB1 protein levels by at least 90%. In some embodiments, a com-position of the disclosure decreases BUB1 protein levels by at least 95%. In some embodiments, a composition of the disclosure decreases BUB1 protein levels by at least 99%.

In some embodiments, a composition of the disclosure decreases BUB1 protein levels to a greater extent than comparator molecules.

In some embodiments, a composition of the disclosure in combination with an autophagy inhibitor decreases BUB1 protein levels. In some embodiments, a composition of the disclosure in combination with an autophagy inhibitor decreases BUB1 protein levels by at least 5%. In some embodiments, a composition of the disclosure in combination with an autophagy inhibitor decreases BUB1 protein levels by at least 10%. In some embodiments, a composition of the dis-closure in combination with an autophagy inhibitor decreases BUB1 protein levels by at least 20%. In some embodiments, a composition of the disclosure in combination with an autophagy inhibitor decreases BUB1 protein levels by at least 30%. In some embodiments, a composition of the disclosure in combination with an autophagy inhibitor decreases BUB1 protein levels by at least 40%. In some embodiments, a composition of the disclosure in combination with an autophagy inhibitor decreases BUB1 protein levels by at least 50%. In some embodiments, a composition of the disclosure in combination with an autophagy inhibitor decreases BUB1 protein levels by at least 60%. In some embodiments, a composition of the disclosure in combination with an autophagy inhibitor decreases BUB1 protein levels by at least 70%. In some embodiments, a composition of the disclosure in combination with an autophagy inhibitor decreases BUB1 protein levels by at least 80%. In some embodiments, a composition of the disclosure in combination with an autophagy inhibitor decreases BUB1 protein levels by at least 90%. In some embodiments, a composition of the disclosure in combination with an autophagy inhibitor decreases BUB1 protein levels by at least 95%. In some embodiments, a composition of the disclosure in combination with an autophagy inhibitor decreases BUB1 protein levels by at least 99%.

In some embodiments, a composition of the disclosure in combination with hydroxychloroquine decreases BUB1 protein levels. In some embodiments, a composition of the disclosure in combination with hydroxychloroquine decreases BUB1 protein levels by at least 5%. In some embodiments, a composition of the disclosure in combination with hydroxychloroquine decreases BUB1 protein levels by at least 10%. In some embodiments, a composition of the disclosure in combination with hydroxychloroquine decreases BUB1 protein levels by at least 20%. In some embodiments, a composition of the disclosure in combination with hydroxychloroquine decreases BUB1 protein levels by at least 30%. In some embodiments, a composition of the disclosure in combination with hydroxychloroquine decreases BUB1 protein levels by at least 40%. In some embodiments, a composition of the disclosure in combination with hydroxychloroquine decreases BUB1 protein levels by at least 50%. In some embodiments, a composition of the disclosure in combination with hydroxychloroquine decreases BUB1 protein levels by at least 60%. In some embodiments, a composition of the disclosure in combination with hydroxychloroquine decreases BUB1 protein levels by at least 70%. In some embodiments, a composition of the disclosure in combination with hydroxychloroquine decreases BUB1 protein levels by at least 80%. In some embodiments, a composition of the disclosure in combination with hydroxychloroquine decreases BUB1 protein levels by at least 90%. In some embodiments, a composition of the disclosure in combination with hydroxychloroquine decreases BUB1 protein levels by at least 95%. In some embodiments, a composition of the disclosure in combination with hydroxychloroquine decreases BUB1 protein levels by at least 99%.

In some embodiments, a composition of the disclosure in combination with hydroxychloroquine decreases BUB1 protein levels to a greater extent than comparator molecules.

In some embodiments, a composition of the disclosure in combination with SBI-0206965 decreases BUB1 protein levels. In some embodiments, a composition of the disclosure in combi-nation with SBI-0206965 decreases BUB1 protein levels by at least 5%. In some embodiments, a composition of the disclosure in combination with SBI-0206965 decreases BUB1 protein levels by at least 10%. In some embodiments, a composition of the disclosure in combination with SBI-0206965 decreases BUB1 protein levels by at least 20%. In some embodiments, a composition of the disclosure in combination with SBI-0206965 decreases BUB1 protein levels by at least 30%. In some embodiments, a composition of the disclosure in combination with SBI-0206965 decreases BUB1 protein levels by at least 40%. In some embodiments, a composition of the disclosure in combination with SBI-0206965 BUB1 protein levels by at least 50%. In some embodiments, a composition of the disclosure in combination with SBI-0206965 decreases BUB1 protein levels by at least 60%. In some embodiments, a composition of the disclosure in combination with SBI-0206965 decreases BUB1 protein levels by at least 70%. In some embodiments, a composition of the disclosure in combination with SBI-0206965 decreases BUB1 protein levels by at least 80%. In some embodiments, a composition of the disclosure in combination with SBI-0206965 decreases BUB1 protein levels by at least 90%. In some embodiments, a composition of the disclosure in combination with SBI-0206965 decreases BUB1 protein levels by at least 95%. In some embodiments, a composition of the disclosure in combination with SBI-0206965 decreases BUB1 protein levels by at least 99%.

In some embodiments, a composition of the disclosure in combination with SBI-0206965 decreases BUB1 protein levels to a greater extent than comparator molecules.

In some embodiments, a composition of the disclosure decreases HER2− breast cancer cell viability in vitro by at least 10%. In some embodiments, a composition of the disclosure decreases HER2− breast cancer cell viability in vitro by at least 20%. In some embodiments, a composition of the disclosure decreases HER2− breast cancer cell viability in vitro by at least 30%. In some embodiments, a composition of the disclosure decreases HER2− breast cancer cell viability in vitro by at least 40%. In some embodiments, a composition of the disclosure decreases HER2− breast cancer cell viability in vitro by at least 50%. In some embodiments, a composition of the disclosure decreases HER2− breast cancer cell viability in vitro by at least 60%. In some embodiments, a composition of the disclosure decreases HER2− breast cancer cell viability in vitro by at least 70%. In some embodiments, a composition of the disclosure decreases HER2− breast cancer cell viability in vitro by at least 80%. In some embodiments, a composition of the disclosure decreases HER2− breast cancer cell viability in vitro by at least 90%. In some embodiments, a composition of the disclosure decreases HER2− breast cancer cell viability in vitro by at least 95%. In some embodiments, a composition of the disclosure decreases HER2− breast cancer cell viability in vitro by at least 99%.

In some embodiments, a composition of the disclosure increases cell death in HER2− breast cancer cells by at least 20%. In some embodiments, a composition of the disclosure increases cell death in HER2− breast cancer cells by at least 30%. In some embodiments, a composition of the disclosure increases cell death in HER2− breast cancer cells by at least 40%. In some embodiments, a composition of the disclosure increases cell death in HER2− breast cancer cells by at least 50%. In some embodiments, a composition of the disclosure increases cell death in HER2− breast cancer cells by at least 60%. In some embodiments, a composition of the disclosure increases cell death in HER2− breast cancer cells by at least 70%. In some embodiments, a composition of the disclosure increases cell death in HER2− breast cancer cells by at least 80%. In some embodiments, a composition of the disclosure increases cell death in HER2− breast cancer cells by at least 90%. In some embodiments, a composition of the disclosure increases cell death in HER2− breast cancer cells by at least 95%. In some embodiments, a composition of the disclosure increases cell death in HER2− breast cancer cells by at least 99%. In some embodiments, treatment with a composition of the disclosure results in PARP cleavage.

In some embodiments, treatment with a composition of the disclosure decreases HER2− breast cancer organoid size. In some embodiments, treatment with a composition of the disclosure decreases HER2− breast cancer organoid size by at least 10%. In some embodiments, treatment with a composition of the disclosure decreases HER2− breast cancer organoid size by at least 20%. In some embodiments, treatment with a composition of the disclosure decreases HER2− breast cancer organoid size by at least 30%. In some embodiments, treatment with a composition of the disclosure decreases HER2− breast cancer organoid size by at least 40%. In some embodiments, treatment with a composition of the disclosure decreases HER2− breast cancer organoid size by at least 50%. In some embodiments, treatment with a composition of the disclosure decreases HER2− breast cancer organoid size by at least 60%. In some embodiments, treatment with a composition of the disclosure decreases HER2− breast cancer organoid size by at least 70%. In some embodiments, treatment with a composition of the disclosure decreases HER2− breast cancer organoid size by at least 80%. In some embodiments, treatment with a composition of the disclosure decreases HER2− breast cancer organoid size by at least 90%. In some embodiments, treatment with a composition of the disclosure decreases HER2− breast cancer organoid size by at least 95%.

In some embodiments, treatment with a composition of the disclosure decreases HER2− breast cancer organoid to a greater extent than the size decrease by a comparator molecule. In some embodiments, treatment with a composition of the disclosure decreases HER2− breast cancer organoid to a greater extent than the size decrease by ribociclib. In some embodiments, treatment with a composition of the disclosure decreases HER2− breast cancer organoid to a greater extent than the size decrease by abemaciclib. In some embodiments, treatment with a composition of the disclosure decreases HER2− breast cancer organoid to a greater extent than the size decrease by palbociclib.

In some embodiments, a composition of the disclosure increases Caspase 3/7 activity compared to an untreated control. In some embodiments, a composition of the disclosure in-creases Caspase 3/7 activity by at least 10% compared to an untreated control. In some embodiments, a composition of the disclosure increases Caspase 3/7 activity by at least 20% compared to an untreated control. In some embodiments, a composition of the disclosure increases Caspase 3/7 activity by at least 30% compared to an untreated control. In some embodiments, a composition of the disclosure increases Caspase 3/7 activity by at least 40% compared to an untreated control. In some embodiments, a composition of the disclosure increases Caspase 3/7 activity by at least 50% compared to an untreated control. In some embodiments, a composition of the dis-closure increases Caspase 3/7 activity by at least 60% compared to an untreated control. In some embodiments, a composition of the disclosure increases Caspase 3/7 activity by at least 70% compared to an untreated control. In some embodiments, a composition of the disclosure in-creases Caspase 3/7 activity by at least 80% compared to an untreated control. In some embodiments, a composition of the disclosure increases Caspase 3/7 activity by at least 90% compared to an untreated control. In some embodiments, a composition of the disclosure increases Caspase 3/7 activity by at least 100% compared to an untreated control. In some embodiments, a composition of the disclosure increases Caspase 3/7 activity by at least 150% compared to an untreated control. In some embodiments, a composition of the disclosure increases Caspase 3/7 activity by at least 200% compared to an untreated control. In some embodiments, a composition of the disclosure increases Caspase 3/7 activity by at least 500% compared to an untreated control. In some embodiments, a composition of the disclosure increases Caspase 3/7 activity by at least 1000% compared to an untreated control.

In some embodiments, treatment with a composition of the disclosure induces apoptotic cell death. In some embodiments, treatment with a composition of the disclosure increases apoptotic cell death relative to an untreated control. In some embodiments, treatment with a composition of the disclosure increases apoptotic cell death by at least 5% relative to an untreated control. In some embodiments, treatment with a composition of the disclosure increases apoptotic cell death by at least 10% relative to an untreated control. In some embodiments, treatment with a composition of the disclosure increases apoptotic cell death by at least 20% relative to an untreated control. In some embodiments, treatment with a composition of the disclosure increases apoptotic cell death by at least 30% relative to an untreated control. In some embodiments, treatment with a composition of the disclosure increases apoptotic cell death by at least 40% relative to an untreated control. In some embodiments, treatment with a composition of the disclosure increases apoptotic cell death by at least 50% relative to an untreated control. In some embodiments, treatment with a composition of the disclosure increases apoptotic cell death by at least 60% relative to an untreated control. In some embodiments, treatment with a composition of the disclosure increases apoptotic cell death by at least 70% relative to an untreated control. In some embodiments, treatment with a composition of the disclosure increases apoptotic cell death by at least 80% relative to an untreated control. In some embodiments, treatment with a composition of the disclosure increases apoptotic cell death by at least 90% relative to an untreated control. In some embodiments, treatment with a composition of the disclosure increases apoptotic cell death by at least 95% relative to an untreated control. In some embodiments, treatment with a composition of the disclosure increases apoptotic cell death by at least 99% relative to an untreated control. In some embodiments, treatment with a composition of the disclosure increases apoptotic cell death by at least 100% relative to an untreated control. In some embodiments, treatment with a composition of the disclosure increases apoptotic cell death by at least 150% relative to an untreated control. In some embodiments, treatment with a composition of the disclosure increases apoptotic cell death by at least 200% relative to an untreated control. In some embodiments, treatment with a composition of the disclosure increases apoptotic cell death by at least 500% relative to an untreated control. In some embodiments, treatment with a composition of the disclosure increases apoptotic cell death by at least 1000% relative to an untreated control.

In some embodiments, treatment with a composition of the disclosure induces non-apoptotic cell death. In some embodiments, treatment with a composition of the disclosure increases non-apoptotic cell death relative to an untreated control. In some embodiments, treatment with a composition of the disclosure increases non-apoptotic cell death by at least 5% relative to an untreated control. In some embodiments, treatment with a composition of the disclosure increases non-apoptotic cell death by at least 10% relative to an untreated control. In some embodiments, treatment with a composition of the disclosure increases non-apoptotic cell death by at least 20% relative to an untreated control. In some embodiments, treatment with a composition of the disclosure increases non-apoptotic cell death by at least 30% relative to an untreated control. In some embodiments, treatment with a composition of the disclosure increases non-apoptotic cell death by at least 40% relative to an untreated control. In some embodiments, treatment with a composition of the disclosure increases non-apoptotic cell death by at least 50% relative to an untreated control. In some embodiments, treatment with a composition of the disclosure increases non-apoptotic cell death by at least 60% relative to an untreated control. In some embodiments, treatment with a composition of the disclosure increases non-apoptotic cell death by at least 70% relative to an untreated control. In some embodiments, treatment with a composition of the disclosure increases non-apoptotic cell death by at least 80% relative to an untreated control. In some embodiments, treatment with a composition of the disclosure increases non-apoptotic cell death by at least 90% relative to an untreated control. In some embodiments, treatment with a composition of the disclosure increases non-apoptotic cell death by at least 95% relative to an untreated control. In some embodiments, treatment with a composition of the disclosure increases non-apoptotic cell death by at least 99% relative to an untreated control. In some embodiments, treatment with a composition of the disclosure increases non-apoptotic cell death by at least 100% relative to an untreated control. In some embodiments, treatment with a composition of the disclosure increases non-apoptotic cell death by at least 150% relative to an untreated control. In some embodiments, treatment with a composition of the disclosure increases non-apoptotic cell death by at least 200% relative to an untreated control. In some embodiments, treatment with a composition of the disclosure increases non-apoptotic cell death by at least 500% relative to an untreated control. In some embodiments, treatment with a composition of the disclosure increases non-apoptotic cell death by at least 1000% relative to an untreated control.

In some embodiments, a composition of the disclosure increases cellular vacuolization. In some embodiments, a composition of the disclosure increases cellular vacuolization by at least 10% relative to an untreated control. In some embodiments, a composition of the disclosure increases cellular vacuolization by at least 20% relative to an untreated control. In some embodiments, a composition of the disclosure increases cellular vacuolization by at least 30% relative to an untreated control. In some embodiments, a composition of the disclosure increases cellular vacuolization by at least 40% relative to an untreated control. In some embodiments, a composition of the disclosure increases cellular vacuolization by at least 50% relative to an untreated control. In some embodiments, a composition of the disclosure increases cellular vacuolization by at least 60% relative to an untreated control. In some embodiments, a composition of the disclosure increases cellular vacuolization by at least 70% relative to an untreated control. In some embodiments, a composition of the disclosure increases cellular vacuolization by at least 80% relative to an untreated control. In some embodiments, a composition of the disclosure increases cellular vacuolization by at least 90% relative to an untreated control. In some embodiments, a composition of the disclosure increases cellular vacuolization by at least 95% relative to an untreated control. In some embodiments, a composition of the disclosure increases cellular vacuolization by at least 99% relative to an untreated control. In some embodiments, a composition of the disclosure in-crease cellular vacuolization by at least 100% relative to an untreated control. In some embodiments, a composition of the disclosure increases cellular vacuolization by at least 150% relative to an untreated control. In some embodiments, a composition of the disclosure increases cellular vacuolization by at least 200% relative to an untreated control. In some embodiments, a composition of the disclosure increases cellular vacuolization by at least 500% relative to an untreated control. In some embodiments, a composition of the disclosure increases cellular vacuolization by at least 1000% relative to an untreated control.

In some embodiments, a composition of the disclosure increases lysosome activity. In some embodiments, a composition of the disclosure increases lysosome activity by at least 5% relative to an untreated control. In some embodiments, a composition of the disclosure increases lysosome activity by at least 10% relative to an untreated control. In some embodiments, a composition of the disclosure increases lysosome activity by at least 20% relative to an untreated control. In some embodiments, a composition of the disclosure increases lysosome activity by at least 30% relative to an untreated control. In some embodiments, a composition of the disclosure increases lysosome activity by at least 40% relative to an untreated control. In some embodiments, a composition of the disclosure increases lysosome activity by at least 50% relative to an untreated control. In some embodiments, a composition of the disclosure increases lysosome activity by at least 60% relative to an untreated control. In some embodiments, a composition of the disclosure increases lysosome activity by at least 70% relative to an untreated control. In some embodiments, a composition of the disclosure increases lysosome activity by at least 80% relative to an untreated control. In some embodiments, a composition of the disclosure increases lysosome activity by at least 90% relative to an untreated control. In some embodiments, a composition of the disclosure increases lysosome activity by at least 95% relative to an untreated control. In some embodiments, a composition of the disclosure increases lysosome activity by at least 99% relative to an untreated control. In some embodiments, a composition of the disclosure increases lysosome activity by at least 100% relative to an untreated control. In some embodiments, a composition of the disclosure increases lysosome activity by at least 150% relative to an untreated control. In some embodiments, a composition of the disclosure increases lysosome activity by at least 200% relative to an untreated control. In some embodiments, a composition of the disclosure increases lysosome activity by at least 500% relative to an untreated control. In some embodiments, a composition of the disclosure increases lysosome activity by at least 1000% relative to an untreated control.

In some embodiments, a composition of the disclosure increases autophagy. In some embodiments, a composition of the disclosure increases autophagy by at least 5% relative to an un-treated control. In some embodiments, a composition of the disclosure increases autophagy by at least 10% relative to an untreated control. In some embodiments, a composition of the disclosure increases autophagy by at least 20% relative to an untreated control. In some embodiments, a composition of the disclosure increases autophagy by at least 30% relative to an untreated control. In some embodiments, a composition of the disclosure increases autophagy by at least 40% relative to an untreated control. In some embodiments, a composition of the disclosure increases autophagy by at least 50% relative to an untreated control. In some embodiments, a composition of the disclosure increases autophagy by at least 60% relative to an untreated control. In some embodiments, a composition of the disclosure increases autophagy by at least 70% relative to an untreated control. In some embodiments, a composition of the disclosure increases autophagy by at least 80% relative to an untreated control. In some embodiments, a composition of the disclosure increases autophagy by at least 90% relative to an untreated control. In some embodiments, a composition of the disclosure increases autophagy by at least 95% relative to an untreated control. In some embodiments, a composition of the disclosure increases autophagy by at least 99% relative to an untreated control. In some embodiments, a composition of the disclosure increases autophagy by at least 100% relative to an untreated control. In some embodiments, a composition of the disclosure increases autophagy by at least 150% relative to an untreated control. In some embodiments, a composition of the disclosure increases autophagy by at least 200% relative to an untreated control. In some embodiments, a composition of the disclosure increases autophagy by at least 500% relative to an untreated control. In some embodiments, a composition of the disclosure increases autophagy by at least 1000% relative to an untreated control.

In some embodiments, the efficacy of a composition of the disclosure is increased following cotreatment with an autophagy inhibitor compared to treatment with the composition alone. In some embodiments, the efficacy of a composition of the disclosure is increased by at least 5% following cotreatment with an autophagy inhibitor compared to treatment with the composition alone. In some embodiments, the efficacy of a composition of the disclosure is increased by at least 10% following cotreatment with an autophagy inhibitor compared to treatment with the composition alone. In some embodiments, the efficacy of a composition of the disclosure is increased by at least 20% following cotreatment with an autophagy inhibitor compared to treatment with the composition alone. In some embodiments, the efficacy of a composition of the disclosure is increased by at least 30% following cotreatment with an autophagy inhibitor compared to treatment with the composition alone. In some embodiments, the efficacy of a composition of the disclosure is increased by at least 40% following cotreatment with an autophagy inhibitor compared to treatment with the composition alone. In some embodiments, the efficacy of a composition of the disclosure is increased by at least 50% following cotreatment with an autophagy inhibitor compared to treatment with the composition alone. In some embodiments, the efficacy of a composition of the disclosure is increased by at least 60% following cotreatment with an autophagy inhibitor compared to treatment with the composition alone. In some embodiments, the efficacy of a composition of the disclosure is increased by at least 70% following cotreatment with an autophagy inhibitor compared to treatment with the composition alone. In some embodiments, the efficacy of a composition of the disclosure is increased by at least 80% following cotreatment with an autophagy inhibitor compared to treatment with the composition alone. In some embodiments, the efficacy of a composition of the disclosure is increased by at least 90% following cotreatment with an autophagy inhibitor compared to treatment with the composition alone. In some embodiments, the efficacy of a composition of the disclosure is increased by at least 95% following cotreatment with an autophagy inhibitor compared to treatment with the composition alone. In some embodiments, the efficacy of a composition of the disclosure is increased by at least 100% following cotreatment with an autophagy inhibitor compared to treatment with the composition alone. In some embodiments, the efficacy of a composition of the disclosure is increased by at least 150% following cotreatment with an autophagy inhibitor compared to treatment with the composition alone. In some embodiments, the efficacy of a composition of the disclosure is increased by at least 200% following cotreatment with an autophagy inhibitor compared to treatment with the composition alone. In some embodiments, the efficacy of a composition of the disclosure is increased by at least 500% following cotreatment with an autophagy inhibitor compared to treatment with the composition alone. In some embodiments, the efficacy of a composition of the disclosure is increased by at least 1000% following cotreatment with an autophagy inhibitor compared to treatment with the composition alone.

In some embodiments, a composition of the disclosure increases cellular senescence. In some embodiments, a composition of the disclosure increases cellular senescence by at least 5% relative to an untreated control. In some embodiments, a composition of the disclosure increases cellular senescence by at least 10% relative to an untreated control. In some embodiments, a composition of the disclosure increases cellular senescence by at least 20% relative to an untreated control. In some embodiments, a composition of the disclosure increases cellular senescence by at least 30% relative to an untreated control. In some embodiments, a composition of the disclosure increases cellular senescence by at least 40% relative to an untreated control. In some embodiments, a composition of the disclosure increases cellular senescence by at least 50% relative to an untreated control. In some embodiments, a composition of the disclosure increases cellular senescence by at least 60% relative to an untreated control. In some embodiments, a composition of the disclosure increases cellular senescence by at least 70% relative to an untreated control. In some embodiments, a composition of the disclosure increases cellular senescence by at least 80% relative to an untreated control. In some embodiments, a composition of the disclosure increases cellular senescence by at least 90% relative to an untreated control. In some embodiments, a composition of the disclosure increases cellular senescence by at least 95% relative to an untreated control. In some embodiments, a composition of the disclosure increases cellular senescence by at least 99% relative to an untreated control. In some embodiments, a composition of the disclosure increases cellular senescence by at least 100% relative to an untreated control. In some embodiments, a composition of the disclosure increases cellular senescence by at least 150% relative to an untreated control. In some embodiments, a composition of the disclosure increases cellular senescence by at least 200% relative to an untreated control. In some embodiments, a composition of the disclosure increases cellular senescence by at least 500% relative to an untreated control. In some embodiments, a composition of the disclosure increases cellular senescence by at least 1000% relative to an untreated control.

In some embodiments, a composition of the disclosure induces T-cell recruiting chemokines. In some embodiments, a composition of the disclosure induces expression of T-cell recruiting chemokines. In some embodiments, a composition of the disclosure induces expression of T-cell recruiting chemokines selected from CCL5, CXCL10, H2D1, and B2M. In some embodiments, a composition of the disclosure induces expression of T-cell recruiting chemokines in a distinct temporal pattern compared to comparator molecules. In some embodiments, a composition of the disclosure induces expression of T-cell recruiting chemokines in a distinct temporal pattern compared to palbociclib. In some embodiments, a composition of the disclosure induces expression of T-cell recruiting chemokines in a distinct temporal pattern compared to abemaciclib. In some embodiments, a composition of the disclosure induces expression of T-cell recruiting chemokines in a distinct temporal pattern compared to ribociclib. In some embodiments, a composition of the disclosure induces expression of T-cell recruiting chemokines in a distinct temporal pattern compared to CDK4/6 inhibitors.

In some embodiments, a composition of the disclosure synergizes with an autophagy inhibitor to induce cell death. In some embodiments, a composition of the disclosure synergizes with an autophagy inhibitor to increase cell death by 5% compared to treatment with the compound alone. In some embodiments, a composition of the disclosure synergizes with an autophagy inhibitor to increase cell death by 10% compared to treatment with the compound alone. In some embodiments, a composition of the disclosure synergizes with an autophagy inhibitor to increase cell death by 20% compared to treatment with the compound alone. In some embodiments, a composition of the disclosure synergizes with an autophagy inhibitor to increase cell death by 30% compared to treatment with the compound alone. In some embodiments, a composition of the disclosure synergizes with an autophagy inhibitor to increase cell death by 40% compared to treatment with the compound alone. In some embodiments, a composition of the disclosure synergizes with an autophagy inhibitor to increase cell death by 50% compared to treatment with the compound alone. In some embodiments, a composition of the disclosure synergizes with an autophagy inhibitor to increase cell death by 60% compared to treatment with the compound alone. In some embodiments, a composition of the disclosure synergizes with an autophagy inhibitor to increase cell death by 70% compared to treatment with the compound alone. In some embodiments, a composition of the disclosure synergizes with an autophagy inhibitor to increase cell death by 80% compared to treatment with the compound alone. In some embodiments, a composition of the disclosure synergizes with an autophagy inhibitor to increase cell death by 90% compared to treatment with the compound alone. In some embodiments, a composition of the disclosure synergizes with an autophagy inhibitor to increase cell death by 95% compared to treatment with the compound alone. In some embodiments, a composition of the disclosure synergizes with an autophagy inhibitor to increase cell death by 99% compared to treatment with the compound alone. In some embodiments, a composition of the disclosure synergizes with an autophagy inhibitor to increase cell death by 100% compared to treatment with the compound alone. In some embodiments, a composition of the disclosure synergizes with an autophagy inhibitor to increase cell death by 150% compared to treatment with the compound alone. In some embodiments, a composition of the disclosure synergizes with an autophagy inhibitor to increase cell death by 200% compared to treatment with the compound alone. In some embodiments, a composition of the disclosure synergizes with an autophagy inhibitor to increase cell death by 500% compared to treatment with the compound alone. In some embodiments, a composition of the disclosure synergizes with an autophagy inhibitor to increase cell death by 1000% compared to treatment with the compound alone.

### EXAMPLES

### Example 1: Study to Evaluate an Oral Pharmaceutical Composition Disclosed Herein for Treating a Disease in a Subject

Summary: This study is a dose escalation study to investigate the safety, tolerability, and PK characteristics of 8-cyclopentyl-2-((4-(4-methylpiperazin-1-yl)phenyl)amino)-7-oxo-7,8-dihydropyrido[2,3-d]pyrimidine-6-carbonitrile (Compound 1) in patients with advanced cancers who have received and failed at least one prior treatment. The primary objective of this study is to assess the safety and tolerability of repeated daily dosing of Compound 1 in patients with relapsed and/or refractory advanced cancers. The secondary objective of this study is to establish a maximum tolerated dose (MTD) and a recommend phase 2 dose (RP2D) of orally administered Compound 1. In addition, the study explores efficacy of Compound 1 in cancer patients.

Study Design: This study is a dose finding study using 3+3 design for dose escalation. Three to six patients are enrolled per dose cohort, followed by up to 12 additional patients at the RP2D. Approximately 36 patients with advanced cancers are enrolled in the study, based on 4 dose levels and an expansion cohort. If additional dose escalations are required to establish the MTD/RP2D, then 3-6 additional patients are added per dose level.

Compound 1 is given in the form of a hard capsule comprising 48.4 mg 8-cyclopentyl-2-((4-(4-methylpiperazin-1-yl)phenyl)amino)-7-oxo-7,8-dihydropyrido[2,3-d]pyrimidine-6-carbonitrile monolactate (Compound 1 salt), equivalent to 40 mg of 8-cyclopentyl-2-((4-(4-methylpiperazin-1-yl)phenyl)amino)-7-oxo-7,8-dihydropyrido[2,3-d]pyrimidine-6-carbonitrile. The initial dose is 40 mg (one capsule) taken once daily for 28 days (one cycle). The dose is taken in the morning, on an empty stomach. Dose increments in the dose escalation 3+3 study are 40 mg of Compound 1 per cycle. Dose levels are 40 mg, 80 mg, 120 mg, 160 mg, etc., until a RP2D/MTD is reached. Each of the first three patients in the first and subsequent cohorts is assessed for dose limiting toxicities (DLT) during the first 28 days of treatment. If no patients experience a DLT, then enrollment to the next cohort begins at the next dose level. If one patient of the first three patients in a cohort experiences a DLT in the first 28 days, then an additional three patients are enrolled to that cohort for a total of six patients. If only one of the six patients in the cohort experiences a DLT in the first 28 days, then enrollment to the next cohort begins at the next dose level. If at any time two or more patients in a cohort experience a DLT in the first 28 days, the cohort is closed. The dose of that cohort is considered non-tolerable and the prior dose level is defined as the MTD. Patients continue in the study until disease progression or intolerance or a decision to discontinue is reached.

Study Objectives and Endpoints: The primary objective of this study is to assess the safety and tolerability of repeated daily dosing of Compound lin patients with relapsed and/or refractory advanced cancers. The primary endpoints include (DLTs, adverse events (AEs), deaths, and other serious AEs.

The secondary objectives of this study are to establish a MTD of Compound 1 salt and a RP2D of orally administered Compound 1 salt and to characterize pharmacokinetics of compound (1) following oral administration in patients with relapsed and/or refractory advanced cancers. Secondary endpoints include maximum plasma concentration (Cmax), area under the plasma concentration time curve (AUC), and half-life (t_{1/2}).

Exploratory objectives of this study are to assess the efficacy of compound (1), by objective responses per RECIST, wherever appropriate for applicable tumors. Assessment of non-Hodgkin's Lymphoma and CNS tumors is by imaging techniques (CT, PET, MRI).

Pharmacokinetics: Blood samples are collected pre- and post-dose on days 1 and 8 of the first cycle and pre-dose on day 1 of cycles 2 and 3 for pharmacokinetic (PK) analysis. Compound (1) concentrations are determined in plasma samples by a validated liquid chromatography-tandem mass spectrometry (LC-MS/MS) assay. Levels of Compound lare determined at specified time points in the PK profile.

The following PK parameters are derived using model-independent analysis: time to reach Cmax (Tmax), Cmax, t_{1/2}, AUC0-t, AUC0-α, CL, and Vss. Descriptive statistics (mean, median, range, standard deviation) for these parameters are provided and summarized by each dose group.

Cmax and Tmax are determined from the plasma concentration-time profile, and t½β is calculated as 0.693/k (where k is the terminal elimination rate constant, calculated by log-linear regression of the terminal portion of the concentration-time profile). AUC0-t is calculated by the linear trapezoidal rule and extrapolated to infinity using k to obtain AUC0-∞.

Pharmacokinetic parameters are calculated from Compound 1 concentration-time data using standard non-compartmental methods as implemented in WinNonlin. The maximum plasma concentration (Cmax) and time to reach Cmax (Tmax) are the observed values. The area under the plasma concentration-time curve (AUC) value is calculated to the last quantifiable sample (AUClast) by use of the linear trapezoidal rule. The AUC values are extrapolated to infinity (AUCinf) by dividing the last quantifiable concentration by the terminal disposition rate constant (λz), which is determined from the slope of the terminal phase of the concentration-time profile. The terminal half-life (T½) is calculated as 0.693 divided by λz. The apparent oral clearance (Cl/F) is calculated by dividing the dose administered by AUCinf.4. Pharmacokinetic data are analyzed by cohort.

Efficacy analysis. The efficacy variable is best overall response (ORR), using RECIST criteria, version 1.1. Objective tumor response is tabulated and summarized by the primary tumor type. If warranted, additional efficacy endpoints, like duration of response or time to progression, are analyzed.

### Example 2: Study to Evaluate PK/PD Results for a Pharmaceutical Composition Disclosed Herein for Treating a Disease in a Subject

Summary: This study is a dose escalation study to investigate the safety, tolerability, and PK characteristics of in-patients with advanced solid tumors who have received and failed at least one prior treatment. The primary objective of this study is to assess the safety and tolerability of repeated daily dosing of 8-cyclopentyl-2-((4-(4-methylpiperazin-1-yl)phenyl)amino)-7-oxo-7,8-dihydropyrido[2,3-d]pyrimidine-6-carbonitrile (Compound 1) in patients with relapsed and/or refractory advanced cancers. The secondary objective of this study is to establish a MT of Compound 1 and RP2D of orally administered Compound 1. In addition, the study explores efficacy of Compound 1 in cancer patients.

Study design: The study includes a treatment period (1 year) and a follow-up period (90 days after the last dose). Subjects are pathologically confirmed to have malignant solid tumors, or advanced (metastatic or unresectable) malignant solid tumors and have previously failed standard treatment (e.g., targeted therapy, chemotherapy, biotherapy, immunotherapy, etc.), as evidenced by disease progression or intolerance toxicity.

The study is divided into two stages, including a dosage escalation and a dose expansion cohort. The first phase is a dose escalation, using 3+3 design to determine MTD and/or RP2D. Three to six patients are enrolled per dose cohort, followed by up to 12 additional patients at the RP2D. Approximately 9-30 patients are enrolled in the first phase. If additional dose escalations are required to establish the MTD/RP2D, then 3-6 additional patients are added per dose level.

Patients receive study drug orally under fasted conditions before breakfast. Compound 1 is administered once daily continuously for 3 weeks with one week break. Safety, tolerability, and dose-limiting toxicity are evaluated after 4 weeks (28 days) of dosing.

Compound 1 is given in the form of a hard capsule comprising 48.4 mg 8-cyclopentyl-2-((4-(4-methylpiperazin-1-yl)phenyl)amino)-7-oxo-7,8-dihydropyrido[2,3-d]pyrimidine-6-carbonitrile monolactate salt, equivalent to 40 mg of 8-cyclopentyl-2-((4-(4-methylpiperazin-1-yl)phenyl)amino)-7-oxo-7,8-dihydropyrido[2,3-d]pyrimidine-6-carbonitrile. The dose (one capsule) is taken in the morning, on an empty stomach. Dose increments in the dose escalation 3+3 study are 40 mg of Compound 1 per cycle. Dose levels are 40 mg, 80 mg, 120 mg, 160 mg, and 200 mg, or until a RP2D/MTD is reached. The highest escalation dose in the study is set at 200 mg. Dose escalation is performed as described in Example 1.

The second stage of the study is a dose expansion stage. The dose expansion stage enrolls 9-12 cancer patients (primarily advanced breast cancer and non-small cell lung cancer patients with HR(+) and HER2(−)). Test procedures are the same as in the dose expansion phase.

Study Objectives and Endpoints: The primary objective of this study is to evaluate the tolerance, safety, and the anti-tumor efficacy of Compound 1 in patients having advanced solid tumors.

The secondary objective of this study is to characterize pharmacokinetics of Compound 1 following oral administration of single and multiple doses of Compound 1 in patients with relapsed and/or refractory advanced cancer. Secondary endpoints include maximum plasma concentration (Cmax), area under the plasma concentration time curve (AUC), and half-life (t½). The study evaluates the efficacy of Compound 1 in patients with solid tumors, including objective response rate ORR, progression-free survival PFS, duration of remission DOR, disease control rate DCR, etc.

Pharmacokinetics: Blood samples are collected pre- and post-dose on days 1 and 8 of the first cycle and pre-dose on day 4 of the first cycle for pharmacokinetic (PK) analysis. Compound 1 PK is determined as described in Example 1.

Efficacy analysis. The efficacy variable of this study is best overall response (ORR), using RECIST criteria, version 1.1. Efficacy analysis includes: (1) Objective Remission Rate (ORR), defined as the proportion of subjects with complete remission (CR) and partial remission (PR) after treatment. (2) Disease Control Rate (DCR), defined as the proportion of subjects with complete remission (CR), partial remission (PR), and disease stabilization (SD) after treatment. (3) Time to remission (DOR), defined as the time from the initial recording of objective remission to the first occurrence of tumor progression, or death from any cause. And (4) Progression free survival (PFS), defined as treatment from initiation to tumor progression or death from any cause.

### Example 3: Dosing Regimen to Evaluate an Oral Pharmaceutical Composition Disclosed Herein with an Aromatase Inhibitor in a Subject

The study is a randomized, double-blind, placebo-controlled, study of 8-cyclopentyl-2-((4-(4-methylpiperazin-1-yl)phenyl)amino)-7-oxo-7,8-dihydropyrido[2,3-d]pyrimidine-6-carbonitrile (Compound 1) in combination with an aromatase inhibitor (e.g., letrozole) versus placebo in combination with an aromatase inhibitor (e.g., letrozole) for patients with estrogen receptor positive advanced or recurrent breast cancer.

Study Design: This study is a treatment response study using 1:1 randomized double-blind study. Patients are randomized into one of two treatment arms: Arm A: (placebo): letrozole-placebo combination therapy; and Arm B (experimental): letrozole-Compound 1 combination therapy. Each cycle in the study is 28 days of treatment with tumors assessed every 12 weeks.

Compound 1 is given in the form of a hard capsule comprising 48.4 mg 8-cyclopentyl-2-((4-(4-methylpiperazin-1-yl)phenyl)amino)-7-oxo-7,8-dihydropyrido[2,3-d]pyrimidine-6-carbonitrile monolactate salt, equivalent to 40 mg of 8-cyclopentyl-2-((4-(4-methylpiperazin-1-yl)phenyl)amino)-7-oxo-7,8-dihydropyrido[2,3-d]pyrimidine-6-carbonitrile. Compound 1 is taken once daily for 28 days (one cycle). Alternatively, Compound 1 is taken once daily continuously for 3 weeks with one week of no administration for a total of 28 days. Treatment continues until progression of disease or unacceptable toxicity. Compound 1 is taken in the morning, on an empty stomach.

Letrozole is given in the form of a 2.5 mg tablet. One 2.5 mg letrozole tablet is taken once on days 1-28 (one cycle). Treatment continues until progression of disease or unacceptable toxicity. Letrozole is taken in the morning, at the same time as either Compound 1 or the placebo.

Primary outcome measures Primary outcome measures are progression-free survival. Progression-free survival is defined as the time from random assignment in a clinical trial to disease progression or death from any cause. The time frame is 24 months.

Secondary Outcome Measures are:
1. Overall survival (OS): time from registration to death due to any cause.
2. Functional Assessment of Cancer Therapy-Breast Cancer (FACT-B). The FACT-B scale is used for evaluating the survival of patients with breast cancer. The scale consists of 36 items covering physical well-being, social/family well-being, emotional well-being, and functional well-being domains. Higher FACT-B score indicates better health.
3. EuroQol five-dimension scale (ED-5Q). The ED-5Q is a widely used multidimensional measure of health-related quality of life. Higher ED-5Q score indicates better health.
4. Overall Response Rate (ORR) according to RECIST.
5. Disease Control Rate (DCR) for at least 12 weeks.
6. Time to First Subsequent Therapy (TFST): time from randomization to first subsequent therapy or death.
7. Progression-Free Survival 2 (PFS2): time from randomization to second objective disease progression or death.
8. Time to Second Subsequent Therapy (TSST): time from randomization to second subsequent therapy or death.
9. Patient Reported Outcomes (PROs) like Quality of Life questionnaire EORTC-QLQ-C30 & EORTC-QLQ-EN24. These are the validated questionnaires to be answered by patients. Results are reported as descriptive and on a scale of 1-10.
10. Number of participants with treatment-related adverse events as assessed by CTCAE v4.0.
11. Compliance in the two treatment arms.
12. Dose reductions/interruptions in the two treatment arms.

Toxicity/efficacy of the various compounds of the disclosure are analyzed and compared.

### Example 4: Dosing Regimen to Evaluate an Oral Pharmaceutical Composition Disclosed Herein with a Progestin in a Subject

The study is a randomized, double-blind, placebo-controlled, study of 8-cyclopentyl-2-((4-(4-methylpiperazin-1-yl)phenyl)amino)-7-oxo-7,8-dihydropyrido[2,3-d]pyrimidine-6-carbonitrile (Compound 1) in combination with a progestin (e.g., megestrol acetate) versus placebo in combination with a progestin (e.g., megestrol acetate) for patients with estrogen receptor positive advanced or recurrent breast cancer.

Study Design: This study is a treatment response study using 1:1 randomized double-blind study. Patients are randomized into one of two treatment arms: Arm A: (placebo): megestrol acetate—placebo combination therapy; and Arm B (experimental): megestrol acetate—Compound 1 combination therapy. Each cycle in the study is 28 days of treatment with tumors assessed every 12 weeks.

Compound 1 is given in the form of a hard capsule comprising 48.4 mg 8-cyclopentyl-2-((4-(4-methylpiperazin-1-yl)phenyl)amino)-7-oxo-7,8-dihydropyrido[2,3-d]pyrimidine-6-carbonitrilemonolactate salt, equivalent to 40 mg of 8-cyclopentyl-2-((4-(4-methylpiperazin-1-yl)phenyl)amino)-7-oxo-7,8-dihydropyrido[2,3-d]pyrimidine-6-carbonitrile. Compound 1 is taken once daily for 28 days (one cycle). Alternatively, Compound 1 is taken once daily continuously for 3 weeks with one week of no administration, for a total of 28 days. Treatment continues until progression of disease or unacceptable toxicity. Compound 1 is taken in the morning, on an empty stomach.

Megestrol acetate is given in the form of 625 mg of an oral suspension per day (5 mL of a 125 mg/mL suspension per day or one teaspoon daily). One 625 mg megestrol acetate dose is taken once on days 1-28 (one cycle). Treatment continues until progression of disease or unacceptable toxicity. Megestrol acetate is taken in the morning, at the same time as either Compound 1 or the placebo.

Primary outcome measures: Primary outcome measures are progression-free survival. Progression-free survival is defined as the time from random assignment in a clinical trial to disease progression or death from any cause. The time frame is 24 months.

Secondary outcome measures: Secondary outcome measures are: 1) overall survival time, which is the length of time from either the date of diagnosis or the start of treatment for cancer, that patients diagnosed with the disease are still alive over a time frame of 24 months; 2) functional assessment of cancer therapy-Breast cancer (FACT-B), which is used to evaluate the survival of patients with breast cancer over a time frame of 24 months. The scale consists of 36 items covering physical well-being, social/family well-being, emotional well-being, and functional well-being domains. Higher FACT-B score indicates better heath; and 3) EuroQol five-dimension scale (ED-5Q) over a time frame of 24 months, which is a multidimensional measure of health-related quality of life. Higher ED-5Q score indicates better health.

Toxicity/efficacy of the various compounds of the disclosure are also analyzed and compared.

### Example 5: Dosing Regimen to Evaluate an Oral Pharmaceutical Composition Disclosed Herein with Fulvestrant in a Subject

The study is a randomized, double-blind, placebo-controlled, study of 8-cyclopentyl-2-((4-(4-methylpiperazin-1-yl)phenyl)amino)-7-oxo-7,8-dihydropyrido[2,3-d]pyrimidine-6-carbonitrile (Compound 1) in combination with a selective estrogen receptor degrader, for example fulvestrant, versus placebo in combination with a selective estrogen receptor degrader, for example fulvestrant for patients with estrogen receptor positive advanced or metastatic breast cancer.

Study Design: This study is a treatment response study using 1:1 randomized double-blind study. Patients with breast cancer are randomized into one of two treatment arms: Arm A: (placebo): fulvestrant-placebo combination therapy; and Arm B (experimental): fulvestrant-Compound 1 combination therapy. Each cycle in the study is 28 days of treatment with tumors assessed every 12 weeks.

Compound 1 is given in the form of a hard capsule comprising 48.4 mg 8-cyclopentyl-2-((4-(4-methylpiperazin-1-yl)phenyl)amino)-7-oxo-7,8-dihydropyrido[2,3-d]pyrimidine-6-carbonitrilemonolactate salt, equivalent to 40 mg of 8-cyclopentyl-2-((4-(4-methylpiperazin-1-yl)phenyl)amino)-7-oxo-7,8-dihydropyrido[2,3-d]pyrimidine-6-carbonitrile. Compound 1 is taken once daily for 28 days (one cycle). Alternatively, Compound 1 is taken once daily continuously for 3 weeks with one week of no administration, for a total of 28 days. Treatment continues until progression of disease or unacceptable toxicity. Compound 1 is taken in the morning, on an empty stomach.

Fulvestrant is given in the form of a prefilled syringe containing 5 mL of an injection solution of 250 mg of fulvestrant for intramuscular injection. The recommended dose is once every 28 days, once at a dose of 500 mg, and on the 15^{th} day after the initial injection, a loading dose of fulvestrant 500 mg is added. Treatment continues until progression of disease or unacceptable toxicity.

Primary outcome measures Primary outcome measures are increase in Progression-Free Survival (PFS) in experimental arm versus comparator arm.

Secondary outcome measures: Secondary outcome measures were: 1) overall survival time, which is the length of time from either the date of diagnosis or the start of treatment for cancer, that patients diagnosed with the disease are still alive over a time frame of 24 months; 2) functional assessment of cancer therapy-Breast cancer (FACT-B), which is used to evaluate the survival of patients with breast cancer over a time frame of 24 months. The scale consists of 36 items covering physical well-being, social/family well-being, emotional well-being, and functional well-being domains. Higher FACT-B score indicates better heath; and 3) EuroQol five-dimension scale (ED-5Q) over a time frame of 24 months, which is a multidimensional measure of health-related quality of life. Higher ED-5Q score indicates better health.

Toxicity/efficacy of the various compounds of the disclosure are analyzed and compared.

### Example 6: Compound 1 is More Effective in Preventing Cancer Cell Growth and Cancer Cell Migration Compared to Comparator 1(Palbociclib)

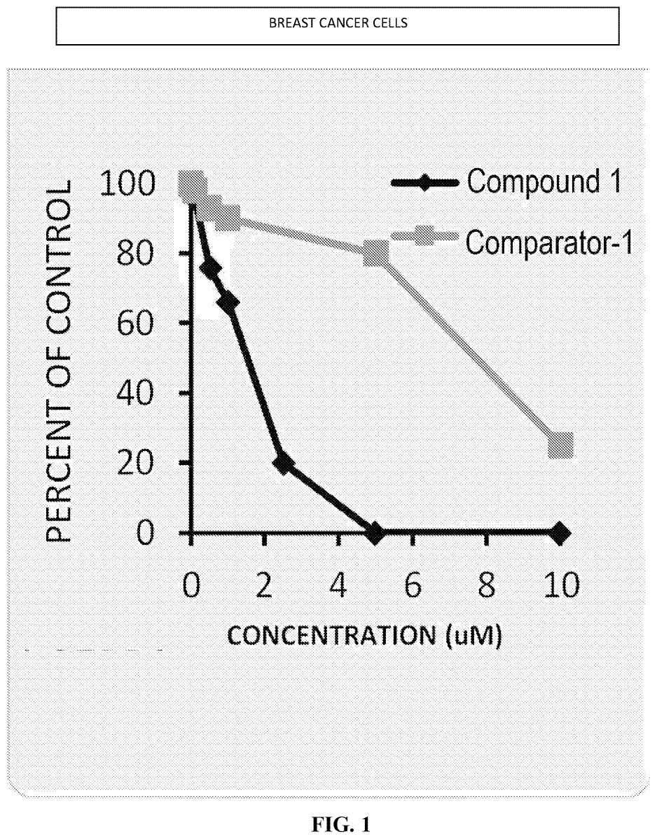

Hs578t cells were treated with 5 μM concentration or 10 μM concentration of Comparator 1 and were grown at 37 degree Celsius for 48 hours. At the end of 48 hours, growth of cells was measured using standard techniques. Results showed, the growth of cells treated with Compound 1 was significantly inhibited compared to cells treated with Comparator 1 (FIG. 1).

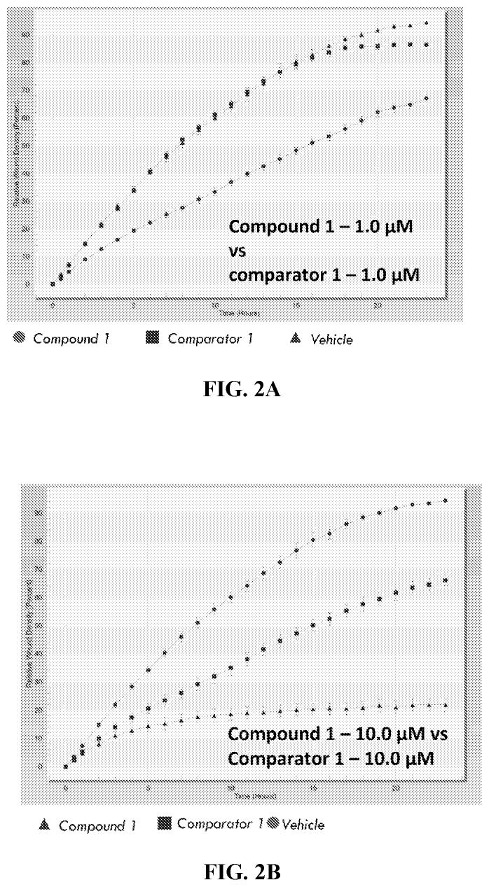

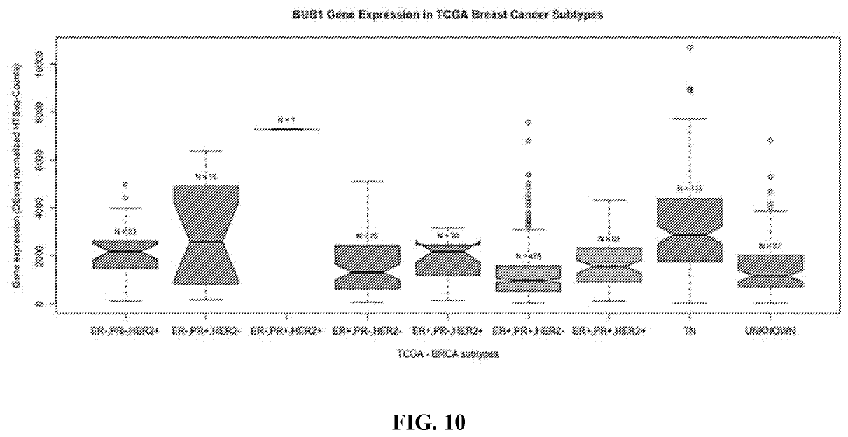

Hs578t and MDA-MB-231 cells were grown on a thin layer on a plastic or glass surface and were treated with Compound 1 or Comparator 1 at 1.0 μM and 10.0 μM concentrations and were allowed to grow for 24 hours. At the end of 24 hours, wound healing assay was performed to study the effect of Compound 1 and Comparator 1 on the coordinated movement of the Hs578t and MDA-MB-231 cell population migration. A cell-free area was created in a confluent monolayer by physical exclusion or by removing the cells from the area through mechanical, thermal or chemical damage. The exposure to the cell-free area induced the cells to migrate into the gap. The rate of gap closure was measured and plotted. FIGS. 2A and 2B showed Compound 1 inhibited cell migration significantly more than Comparator 1 at both at 1.0 μM (FIG. 2A) and 10.0 μM (FIG. 2B) concentrations.

### Example 7: Cellular Thermal Shift Assay (CETSA) Profiling ofMDA-MB-2341 Cells Treated with Compound 1 Compared to Comparator 1

A study was designed to identify cellular targets specific to Compound 1 over a c Comparator 1).

Triple negative breast cancer (TNBC) MDA-MB-231 lysates were treated with 2 μM Compound 1, 2 μM Comparator 1, 20 μM Compound 1, or 20 μM Comparator 1. Following treatment, cell lysates were thermally treated in a thermal gradient profile across a temperature range. Following the thermal shift, cell lysates were spun by centrifuge to pellet aggregate protein and the supernatant withdrawn. The supernatant was then denatured in buffer and heated to denature, reduce, and alkylate the supernatant proteins. Following heating and cooling to room temperature, cold acetone was added to the supernatant samples and proteins precipitated.

TNBC MDA-MB-231 cells were grown at 37° C. in 5% CO₂ and suspended in warm media and split into 4 equal volumes. Cell volumes were treated with 2 μM Compound 1, 2 μM Comparator 1, 20 μM Compound 1, or 20 μM Comparator 1. Cells were incubated and then pelleted. Following pelleting, the cells were washed, resuspended, and heated in a thermal gradient. Following heating, the samples were flash frozen and lysed in lysis buffer, agitated by vortex, and sonicated prior to centrifugation. The supernatant was collected and denaturation buffer added. Cold acetone was then added to the cell samples, and protein collected by centrifugation. Pellets were then washed twice with cold acetone and the washed pellets prior to labeling.

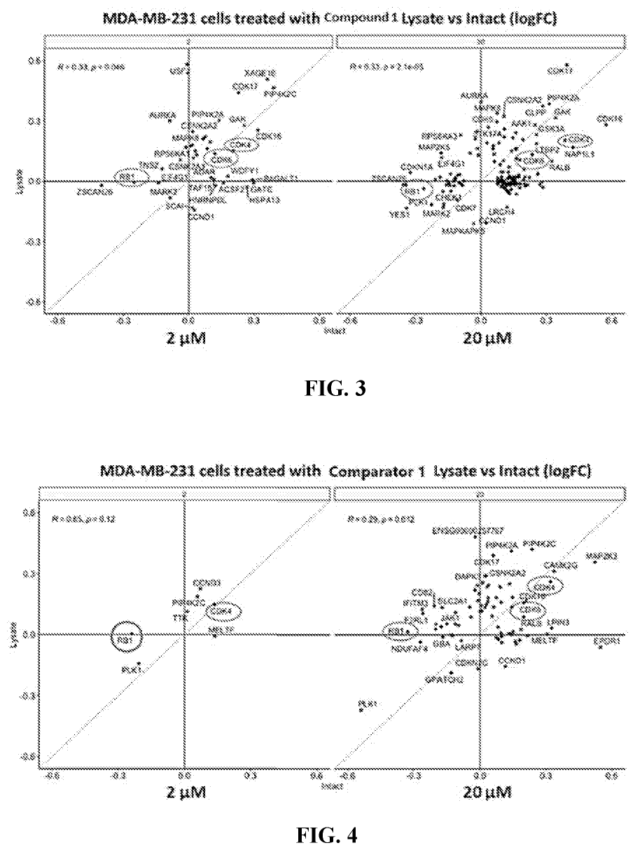

The intact cell protein samples and lysate protein samples were then digested and labeled for mass spectrometry analysis according to the manufacturer's recommended protocols. Digested and labeled protein samples were analyzed by mass spectrometry proteomic analysis and the resulting protein targets were analyzed and plotted. FIG. 3 shows the cellular thermal shift assay data of MDA-MB-231 cell lysates treated with Compound 1 compared to MDA-MB-231 intact cells treated with Compound, with the log(fold chain) change in the MDA-MB-231 cell lysate targets compared to the intact MDA-MB-231 cells treated with 2 μM and 20 μM Compound 1. FIG. 4 shows the cellular thermal shift assay data of MDA-MB-231 cell lysates treated with Comparator 1 compared to MDA-MB-231 intact cells treated with Compound, with the log(fold chain) change in the MDA-MB-231 cell lysate targets compared to the intact MDA-MB-231 cells treated with 2 μM and 20 μM Comparator 1.

TABLE 1 shows a list of 29 targets unique to Compound 1 in MDA-MB-231 cell lysates compared to comparator 1. TABLE 2 shows a list of 148 targets unique to Compound 1 in MDA-MB-231 intact cells compared to comparator 1, and TABLE 3 shows a list of 5 targets that are present in both MDA-MB-231 cell lysates and intact cells and unique to Compound 1. Comparator 1 was not found to affect any of the targets identified in Tables 1-3 (FIG. 4).

TABLE 1
Targets unique to Compound 1 in MDA-MB-231
cell lysates compared to comparator 1

| Gene | Group |
|---|---|
| CDC42BPB | Destabilization |
| CHEK1 | Destabilization |
| DGCR6 | Destabilization |
| MAPKAPK5 | Destabilization |
| TBK1 | Destabilization |
| UVSSA | Destabilization |
| ZNF260 | Destabilization |
| AAK1 | Stabilization |
| ATAT1 | Stabilization |
| AURKA | Stabilization |
| BMP2K | Stabilization |
| BUB1 | Stabilization |
| CDK2 | Stabilization |
| CDK5 | Stabilization |
| CPQ | Stabilization |
| EPHA2 | Stabilization |
| FKBP8 | Stabilization |
| GSK3A | Stabilization |
| GSK3B | Stabilization |
| LIMK1 | Stabilization |
| MAP11 | Stabilization |
| PER3 | Stabilization |
| SLK | Stabilization |
| STK17A | Stabilization |
| STK17B | Stabilization |
| TFEB | Stabilization |
| TGFBR2 | Stabilization |
| USF2 | Stabilization |
| ZCRB1 | Stabilization |

TABLE 2
Targets unique to Compound 1 in MDA-MB-231
intact cells compared to comparator 1

| Gene | Group |
|---|---|
| ACAA1 | Destabilization |
| AIFM2 | Destabilization |
| ANP32B | Destabilization |
| AP2A1 | Destabilization |
| AP2M1 | Destabilization |
| AP2S1 | Destabilization |
| AVEN | Destabilization |
| BOD1L1 | Destabilization |
| CDK12 | Destabilization |
| CDK7 | Destabilization |
| CDKN1A | Destabilization |
| CHEK1 | Destabilization |
| COG8 | Destabilization |
| DECR1 | Destabilization |
| EIF4G1 | Destabilization |
| FOS | Destabilization |
| FOSB | Destabilization |
| FOXK1 | Destabilization |
| LYN | Destabilization |
| LYPLA2 | Destabilization |
| MAP2K5 | Destabilization |
| MAP3K20 | Destabilization |
| MARK2 | Destabilization |
| MICALL2 | Destabilization |
| NPM1 | Destabilization |
| PUS7L | Destabilization |
| RPS6KA3 | Destabilization |
| SART3 | Destabilization |
| SCP2 | Destabilization |
| SHCBP1 | Destabilization |
| SMAD1 | Destabilization |
| SNX30 | Destabilization |
| SNX6 | Destabilization |
| STK3 | Destabilization |
| TLE5 | Destabilization |
| YES1 | Destabilization |
| ZNF608 | Destabilization |
| AAK1 | Stabilization |
| ADAR | Stabilization |
| AGRN | Stabilization |
| AHCTF1 | Stabilization |
| APLP2 | Stabilization |
| APP | Stabilization |
| ARRB2 | Stabilization |
| AXL | Stabilization |
| BMP2K | Stabilization |
| CAVIN1 | Stabilization |
| CAVIN3 | Stabilization |
| CBX5 | Stabilization |
| CCN1 | Stabilization |
| CDC5L | Stabilization |
| CDK17 | Stabilization |
| CELF1 | Stabilization |
| CERS2 | Stabilization |
| CLPP | Stabilization |
| CLUH | Stabilization |
| COL2A1 | Stabilization |
| CPD | Stabilization |
| CRAT | Stabilization |
| CSNK2A2 | Stabilization |
| CST3 | Stabilization |
| DBNDD2 | Stabilization |
| DDX21 | Stabilization |
| DHX9 | Stabilization |
| DNAJC13 | Stabilization |
| ECE1 | Stabilization |
| ELAVL1 | Stabilization |
| ELL | Stabilization |
| ESYT1 | Stabilization |
| FASTKD5 | Stabilization |
| FBRS | Stabilization |
| FGFRL1 | Stabilization |
| FUBP3 | Stabilization |
| GAK | Stabilization |
| GALNT7 | Stabilization |
| GCAT | Stabilization |
| GSK3A | Stabilization |
| GSK3B | Stabilization |
| GSKIP | Stabilization |
| HNRNPA0 | Stabilization |
| HNRNPA1 | Stabilization |
| HNRNPA2B1 | Stabilization |
| HNRNPA3 | Stabilization |
| HNRNPC | Stabilization |
| HNRNPL | Stabilization |
| HNRNPR | Stabilization |
| HNRNPU | Stabilization |
| HNRNPU | Stabilization |
| HNRNPUL2 | Stabilization |
| HSPA13 | Stabilization |
| HSPG2 | Stabilization |
| IFT52 | Stabilization |
| IGFBP4 | Stabilization |
| IGFBP7 | Stabilization |
| ILF3 | Stabilization |
| INCENP | Stabilization |
| JMY | Stabilization |
| LAMA5 | Stabilization |
| LAMC1 | Stabilization |
| LAMTOR2 | Stabilization |
| LEO1 | Stabilization |
| LMAN2 | Stabilization |
| LOXL2 | Stabilization |
| LRCH4 | Stabilization |
| LRPPRC | Stabilization |
| LRRC59 | Stabilization |
| LTBP2 | Stabilization |
| MAMDC2 | Stabilization |
| MAN2A1 | Stabilization |
| MATR3 | Stabilization |
| METTL18 | Stabilization |
| MLEC | Stabilization |
| MPHOSPH8 | Stabilization |
| MRC2 | Stabilization |
| MRPL55 | Stabilization |
| MTG1 | Stabilization |
| NAP1L1 | Stabilization |
| NAPG | Stabilization |
| NRP1 | Stabilization |
| PHF8 | Stabilization |
| PIK3C3 | Stabilization |
| PIK3R4 | Stabilization |
| PIP4K2A | Stabilization |
| PIP4K2B | Stabilization |
| PIP4P2 | Stabilization |
| PRPF4B | Stabilization |
| PTX3 | Stabilization |
| RAE1 | Stabilization |
| RBM34 | Stabilization |
| RIPK 1 | Stabilization |
| RNF5 | Stabilization |
| RRAS2 | Stabilization |
| RSF1 | Stabilization |
| SCAF1 | Stabilization |
| SGPP1 | Stabilization |
| SIK2 | Stabilization |
| SMU1 | Stabilization |
| SND1 | Stabilization |
| STK10 | Stabilization |
| STX5 | Stabilization |
| SYNCRIP | Stabilization |
| SYNE2 | Stabilization |
| TAF15 | Stabilization |
| TIMP2 | Stabilization |
| TTK | Stabilization |
| UFL1 | Stabilization |
| WTAP | Stabilization |
| ZFR | Stabilization |

TABLE 3
Targets that are present in both MDA-MB-231 cell
lysates and intact cells and unique to Compound 1

| Gene | Group |
|---|---|
| CHEK1 | Destabilization |
| AAK1 | Stabilization |
| BMP2K | Stabilization |
| GSK3A | Stabilization |
| GSK3B | Stabilization |

Overall, the data identify protein targets unique to Compound 1 in comparison to Comparator 1.

### Example 8: Phosphoproteomic Analysis ofMDA-MB-231 Cells Treated with Compound 1 Compared to Comparator 1

A series of experiments was designed to identify kinase targets unique to Compound 1 in comparison to Comparator 1.

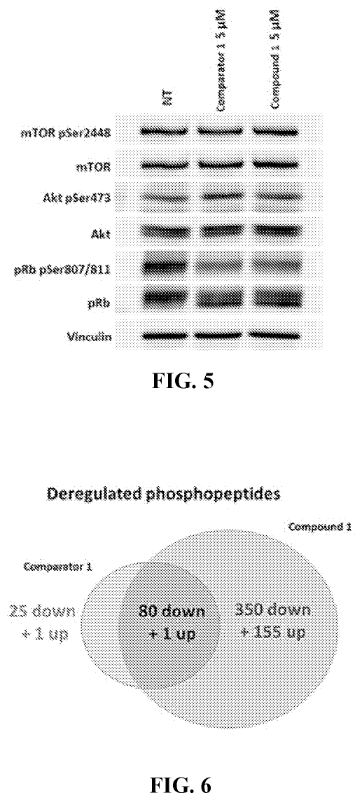

TNBC MD-MDA-231 cell lysates were treated with DMSO, 5 μM Compound 1, or 5 μM Comparator 1 for 2 hours. Following treatment, a sample of each of the three treatments was analyzed by Western Blot for expression of phosphoprotein targets. FIG. 5 shows the western blot for mTOR, mTOR pSer2448, Akt, Akt pSer473, pRb, and pRb pSer807/811 with vinculin used as a loading control. Treatment with Compound 1 or Comparator 1 resulted in decreased phosphorylation of pRb at Ser807/811 and decreased pRb levels relative to the non-treated DMSO control.

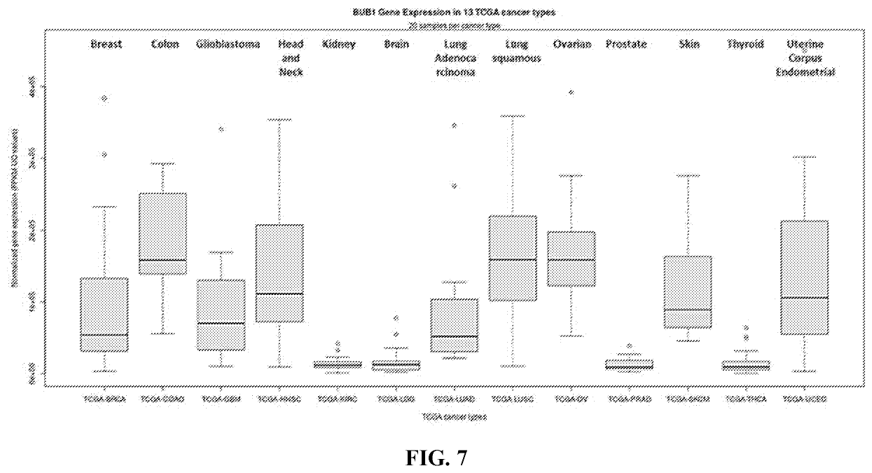

In addition to western blot analysis, the cell lysate samples underwent phosphoproteomic analysis. FIG. 6 shows a summary of the deregulated phosphopeptides following Compound 1 or Comparator 1 treatment. Following Compound 1 treatment, a total of 350 phosphopeptides were downregulated and 155 phosphopeptides were upregulated. 25 phosphopeptides were downregulated and 1 phosphopeptide was upregulated following Comparator 1 treatment. A total of 80 phosphopeptides were downregulated when treated with Compound 1 or Comparator 1. 1 phosphopeptide was upregulated when treated with Compound 1 or Comparator 1. 350 phosphopeptides were downregulated when treated with Compound 1 but were unaffected by Comparator 1 and 155 phosphopeptides were upregulated when treated with Compound 1 but were unaffected by Comparator 1. A summary of Compound 1 and Comparator 1 specific phosphopeptides targets following Compound 1 and Comparator 1 treatment, based on T-test statistic test (p<0,05) is listed in TABLE 4.

TABLE 4
Compound 1 and Comparator 1 specific phosphopeptides targets

| Phosphopeptide | |
|---|---|
| Targets | Significance |
| CSNK1A1 | Statistically |
| MAP3K11 | significant |
| TESK1 | both for |
| ATR | Compound |
| | 1 and |
| | Comparator |
| | 1 |
| PRKD1 | Statistically |
| ULK1 | significant |
| RAF1 | only for |
| MAP2K2 | Compound |
| CAMK2D | 1 treated |
| LYN | cells and |
| PRKD2 | between the |
| AKT2 | two |
| TLK1 | treatments |
| GTF2F1 | |
| STK3 | |
| CAMK1 | |
| LATS1 | |
| BRAF | |
| NUAK1 | |
| HCK | |
| BUB1 | |
| ARAF | |
| MAP2K5 | |
| CDK16 | |
| MAPK7 | |
| CDK7 | Statistically |
| | significant |
| | only for |
| | Comparator |
| | 1 treated |
| | cells but not |
| | between |
| | treatments |

Overall, the data indicate that Compound 1 treatment results in differential regulation of phosphopeptides in MDA-MB-231 cell lysates compared to Comparator 1.

### Example 9: Analysis of BUBI Expression in HER2− Breast Cancer and Uterine Corpus Endometrial Carcinomas (UCEC)

A series of bioinformatic analyses on human breast cancer and uterine corpus endometrial carcinoma (UCEC) patient data was performed to analyze BUB1 expression levels and survival.

Human breast cancer and UCEC data were obtained from The Cancer Genome Atlas (TCGA) and The National Cancer Institute's Clinical Proteomic Tumor Analysis Consortium (CPTAC). From the obtained data, the normalized gene expression for BUB1 from 13 cancer types was determined with 20 samples per cancer type analyzed. FIG. 7 shows a plot of the normalized BUB1 gene expression (FPKM-UQ values) for each cancer type. Of the cancer types assessed, colon, head and neck, lung squamous, ovarian, skin, and uterine corpus endometrial cancers had among the highest normalized BUB1 gene expression relative to the other cancer types. Breast, glioblastoma, and lung adenocarcinoma cancers had normalized BUB1 gene expression values less than the highest cancers, but had higher BUB1 expression than kidney, brain, prostate, and thyroid cancers.

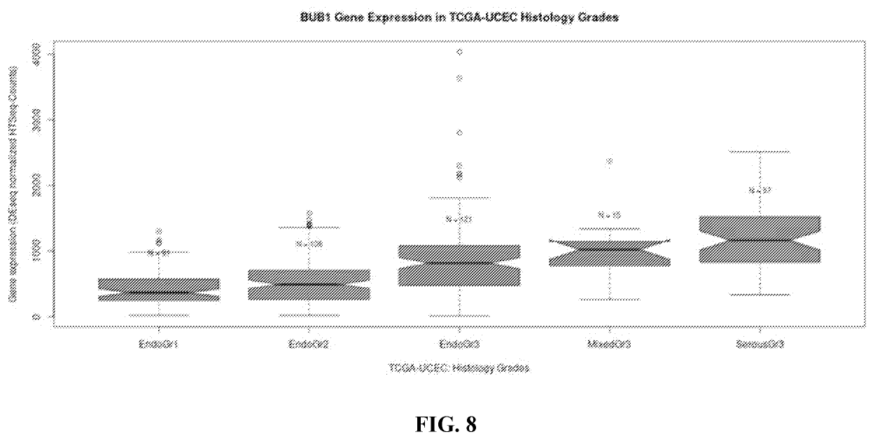

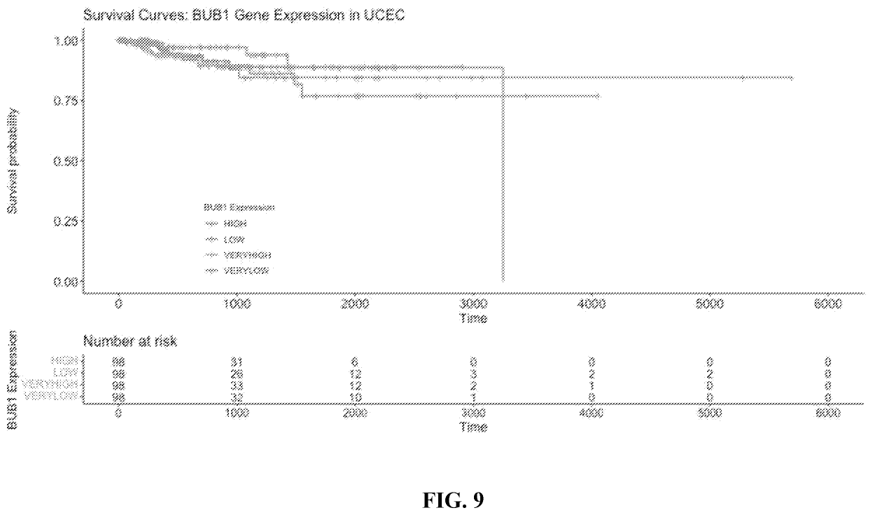

UCEC cancers had among the highest normalized BUB1 gene expression across the 13 cancer types analyzed. To determine whether BUB1 expression was associated with severity, the normalized expression of BUB1 was determined across 5 TCGA-UCEC histology grades. FIG. 8 shows the normalized expression of BUB1 across the different histology grades, with increased BUB1 gene expression observed in EndoGr3, MixedGr3, and SeriousGR3 histologically graded cancers relative to EndoGr1 and EndoGr2 cancers. To determine whether BUB1 gene expression affects survival, the survival probability over time was determined for patients with UCEC with high, very high, low, and very low expression of BUB1. FIG. 9 shows the survival probability over time for UCEC stratified based on BUB1 expression. UCEC patients with very high expression of BUB1 exhibited a lower survival probability compared to those having other BUB1 expression levels.

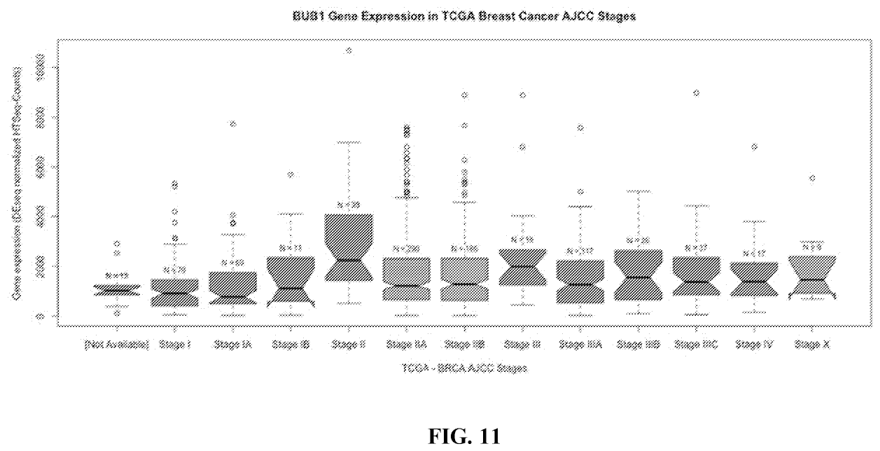

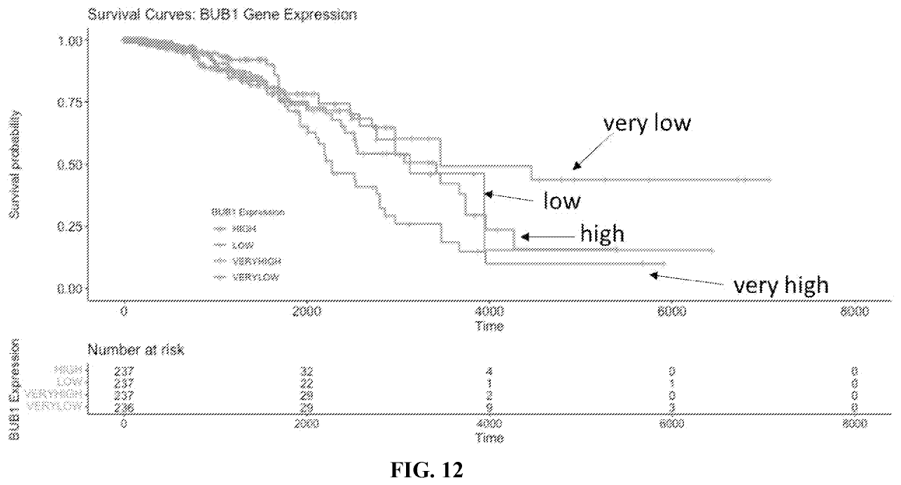

To determine whether BUB1 expression varied across TCGA breast cancer subtypes, the normalized gene expression of BUB1 was determined for ER−, PR+, HER2−; ER+, PR−, HER2−, ER+, PR+, HER2−, Triple Negative (TN), and unknown breast cancer subtypes. FIG. 10 shows the normalized BUB1 gene expression across the different breast cancer subtypes. ER−, PR+, HER2−; and TN subtypes had the highest relative expression of BUB1 compared to the other subtypes, with high intrasample variability observed. Following ER−, PR+, HER2− and TN cancers, ER+, PR−, HER2− had the next highest BUB1 expression followed by ER+PR+HER2− and unknown subtypes. To determine whether BUB1 expression varied across the breast cancer stage, the normalized BUB1 gene expression was determined across American Joint Committee on Cancer (AJCC) stages: Stage I, Stage IA, Stage IB, Stage II, Stage IIA, Stage IIB, Stage III, Stage IIIA, Stage IIIB, Stage IIIC, Stage IV, and Stage X. FIG. 11 shows the normalized BUB1 gene expression level across the different AJCC stages. Stage II breast cancer had the highest normalized BUB1 expression across the stages. BUB1 expression was observed to be relatively equal across the different stages, with high intrasample variability. To determine whether BUB1 expression affects survival, the survival probability over time was determined for breast cancer patients with high, very high, low, and very low expression of BUB1. FIG. 12 shows the survival probability for breast cancer stratified based on BUB1 expression. Patients with breast cancer with very high levels of BUB1 exhibited a lower survival probability compared to those having other BUB1 expression levels.

Overall, the data indicate that BUB1 expression is elevated in certain cancers, including UCEC and breast cancer, with increased BUB1 expression observed in UCEC cases with higher grade histology, and very high expression of BUB1 is associated with a lower survival probability for UCEC and breast cancers.

### Example 10: Prediction of Binding-Site Conformation of Compound 1 in BUB1 and Effect of Compound 1 Treatment on BUB1 Protein Levels

A series of studies was designed to assess the effect of Compound 1 treatment on BUB1.

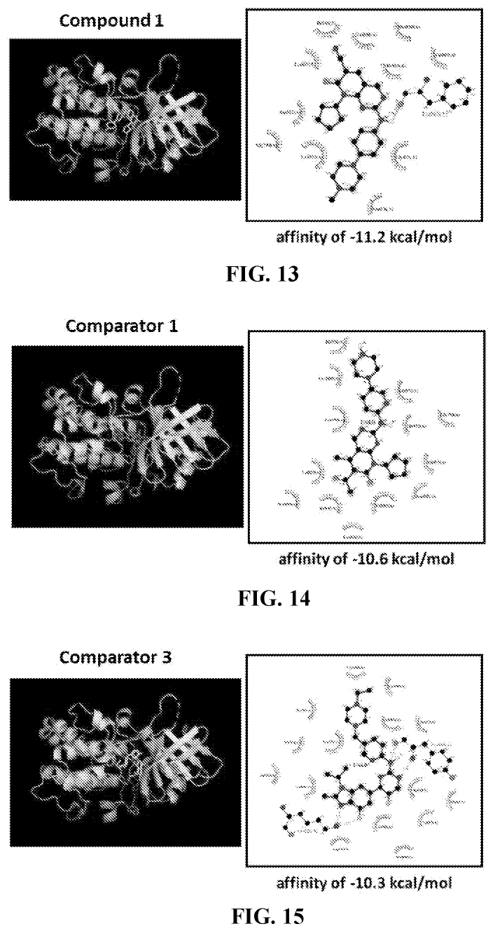

To determine whether Compound 1 binds to BUB1, molecular docking experiments were performed to predict the binding conformations, binding site, and binding affinity of Compound 1, Comparator 1, and Comparator 3 (abemaciclib) with BUB1. FIGS. 13-15 show the predicted binding site confirmations of the three different molecules. The result suggests that Compound 1 binds in BUB1 with a different confirmation and with different binding affinity than that of Comparator 1 or Comparator 3. FIG. 13 shows the predicted binding-conformation (left panel) of and binding site (right panel) of Compound 1 to BUB1. FIG. 14 shows the predicted binding-conformation (left panel) of and binding site (right panel) of Comparator 1 to BUB1. FIG. 15 shows the predicted binding-conformation (left panel) of and binding site (right panel) of Comparator 3 to BUB1.

TABLE 5 lists the binding site interactions for Compound 1, Comparator 1, and Comparator 3. The results predict that Compound 1 exhibits a greater degree of hydrogen bonding interactions with BUB1 residues than does either of Comparator 1 or Comparator 3, and has an overall different hydrophobic interaction profile than both Comparators exhibit.

TABLE 5
Binding site interactions for Compound 1,
Comparator 1, and Comparator 3

| | | | Com- | Com- | Com- |
|---|---|---|---|---|---|
| Residue | Restype | Interaction | pound 1 | parator 3 | parator 1 |
| 821* | LYS | Hydrogen bond | # | # | |
| 869 | TYR | Hydrogen bond | # | # | |
| 869 | TYR | Hydrogen bond | # | | |
| 946 | ASP | Hydrogen bond | # | | |
| 791 | HIS | Hydrophobic | | # | # |
| 793* | LEU | Hydrophobic | # | # | # |
| 794* | GLY | Hydrophobic | # | # | # |
| 801* | VAL | Hydrophobic | # | # | # |
| 819 | VAL | Hydrophobic | # | # | # |
| 821 | LYS | Hydrophobic | # | | # |
| 850 | MET | Hydrophobic | # | # | # |
| 867 | GLU | Hydrophobic | | # | |
| 872 | GLY | Hydrophobic | # | # | # |
| 873 | THR | Hydrophobic | # | # | # |
| 876 | ASN | Hydrophobic | # | | |
| 921 | ASP | Hydrophobic | # | # | # |
| 922 | ASN | Hydrophobic | | | # |
| 924 | ILE | Hydrophobic | # | # | |
| 945 | ILE | Hydrophobic | # | # | # |
| 946 | ASP | Hydrophobic | | # | |
| 1080 | CYS | Hydrophobic | | # | |
| 1081 | LYS | Hydrophobic | | # | # |

*Involved in the stabilization of the ATP molecule

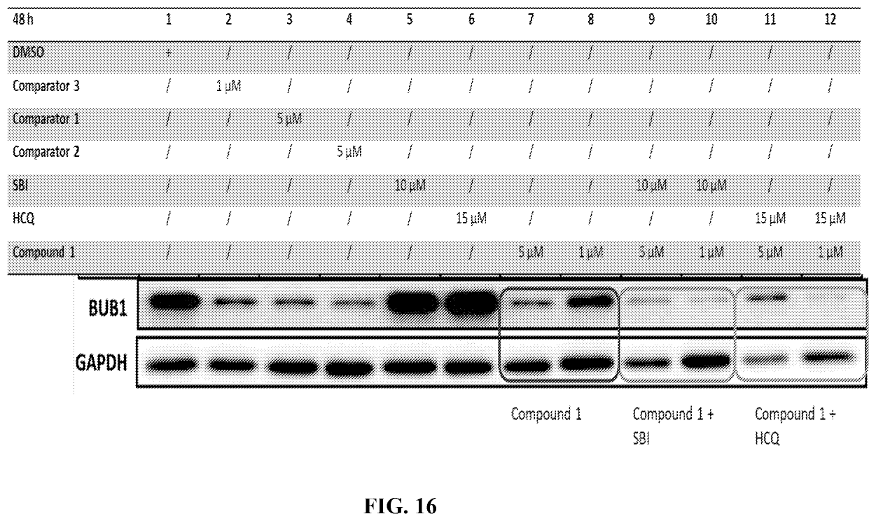

To determine the effect of Compound 1 treatment on BUB1 protein levels, MDA-MB-231 cells were treated with varying concentrations of, Comparator 1, Comparator 2 (ribociclib), Comparator 3, or Compound 1 alone or in combination with the autophagy inhibitors hydroxychloroquine (HCQ) and SBI-0206965 (SBI) for 48 hours and the total cell lysates analyzed for BUB1 protein levels by western blot. FIG. 16 shows the treatment conditions and western blot for BUB1 levels. Treatment with Comparator 1, Comparator 2, Comparator 3, or Compound 1 decreased BUB1 protein levels relative to the DMSO control. Treatment of MDA-MB-231 cells with Compound 1 and HCQ or Compound 1 and SBI decreased BUB1 levels to a greater extent than Compound 1 treatment did alone and to a greater extent than did the Comparators. The results suggest that autophagy inhibition could enhance the effects of Compound 1 treatment on BUB1 levels.

Overall, the data indicate that Compound 1 binds to BUB1 via a different conformation than comparators did and that autophagy inhibition can enhance the BUB1-lowering effects of Compound 1.

### Example 11: Analysis of In Vitro Efficacy of Compound 1 in HER2− Breast Cancer Cell Lines

A series of experiments was designed to assess the in vitro efficacy of Compound 1 in HER2− breast cancer cells.

A panel of HER2− breast cancer cell lines: T-47D, BT-474, MDA-MB-453, MDA-MB-468, HCC-1937, Hs578T, and MDA-MB-231, was treated for 48 hours with 5 μM Compound 1 and the cell viability was measured using the MTT assay. TABLE 6 summarizes the cell lines, and the change in viability after 48 hours of treatment with Compound 1. Among the 6 cell lines assessed, only HCC-1937 and Hs578T cells had a decrease in cell viability less than 50%, while the other cell lines all had viability decreases at or greater than 50%.

TABLE 6: Cell lines and the change in viability after 48 h of treatment with Compound 1

To the in vitro efficacy of Compound 1 on inducing cell death in HER2− breast cancer

| | | | 48 h-cell | |
|---|---|---|---|---|
| | Cancer | | viability | |
| Cell line | Type | Treatment | decrease | Known mutations |
| T-47D | HR+ | Compound 1 | 60% | PIK3CA H1047R; TP53 |
| | HER2− | | | L194F |
| BT-474 | HR+ | Compound 1 | 50% | MAPK1 H61Q; PIK3CA |
| | HER2+ | | | K111N; TP53 E285K |
| MDA-MB- | TNBC | Compound 1 | 50% | AR Q868H; CDH1 W638*; |
| 453 | | | | KRAS G13D; PIK3CA |
| | | | | H1047R; PTEN Q307K |
| MDA-MB- | TNBC | Compound 1 | 65% | RB1c.265_2787del2523; |
| 468 | | | | TP53 R273H; PTEN |
| | | | | c.253 + 1G > T |
| HCC-1937 | TNBC | Compound 1 | 20% | BRCA1 Q1756Pfs*74; TP53 |
| | | | | R306*; RB1 delT738-775; |
| | | | | PTEN deletion |
| Hs578T | TNBC | Compound 1 | 40% | CDKN2A deletion; HRAS |
| | | | | G12D; MT-CYB A125T; |
| | | | | TP53 V157F |
| MDA-MB- | TNBC | Compound 1 | 60% | BRAF G464V; CDKN2A |
| 231 | | | | deletion; KRAS G13D; TP53 |
| | | | | R280K; TERT c.1-124C > T |

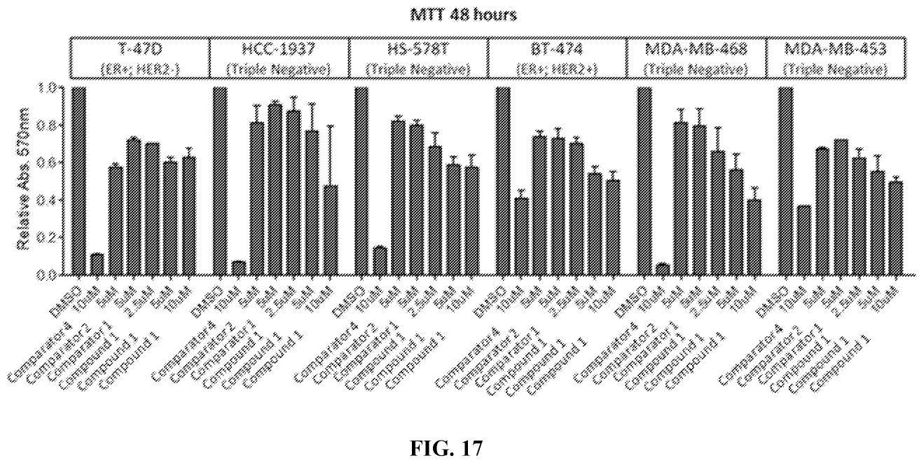

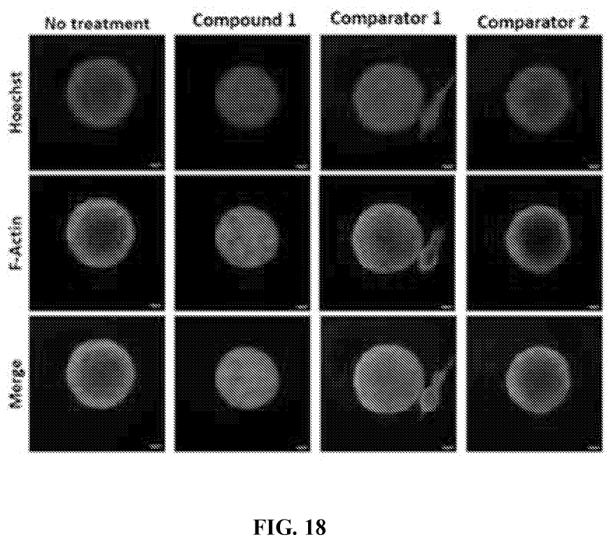

cell lines was compared to Comparator 1, Comparator 2, and Comparator 4 (bortezomib). T-47D, HCC-1937, Hs-578T, MDA-MVB-463, and MDA-MB-453 cells were treated with: DMSO, Comparator 4 (10 μM) Comparator 2 (5 μM), Comparator 1 (5 μM), Compound 1 (2.5 μM, 5.0 μM, and 10 μM) for 48 hrs and the cell viability assessed using the MTT assay. FIG. 17 shows the viabilities of the different cell lines. 10 μM Comparator 4 induced cell death to the greatest extent compared to Compound 1 and Comparators 1 and 2 for all the cell lines assessed. A dose-dependent effect was observed with Compound 1 for HC-1937, Hs-578T, BT-474, MDA-MB-468, and MIDA-MB-453 cell lines with increasing doses of Compound 1 resulting in increased cell death. Compound 1 exhibited greater potency in inducing cell death compared to both Comparator 1 and Comparator 2 for the same cell lines. To determine whether the addition of autophagy inhibitors increased cell death, MDA-MD-231, MDA-MB-468, and HCC-1937 cells were treated with Compound 1, Compound 1+hydroxychloroquine (HCQ), Compound 1+SB+-0206965 (SBI); Comparator 3, Comparator 3+HCQ, Comparator 3+SBI; and Comparator 1, Comparator 1+HCQ, and Comparator 1+SBI for 72 hours. The effects on the different treatment conditions on BUB 1 levels and PARP cleavage were also assessed. TABLE 7 summarizes the effects of the different treatments. For both MNA-MB-231 cells, Compound 1 alone or with HCQ or SBI decreased BUB1 levels and cleaved PARP with over 7500 decreases in cell viability observed for all treatments except Compound 1+SBI in MMA-MB-231 cells. The effects of Compound 1 with or without autophagy inhibitors was less pronounced in HCC-1937 cells. For all three cell lines assessed, Compound 1 treatment decreased cell viability to a greater extent than both Comparator 1 and Comparator 3. The combination of autophagy inhibitors with Compound 1 provided slightly enhanced activity compared to Compound 1 alone. Greater magnitudes of cell viability decrease were observed with the +HCQ and +SB conditions compared to the Compound 1 treatment alone.

TABLE 7

| | | | | | 72 h-cell |
|---|---|---|---|---|---|
| | Cancer | | | PARP | viability |
| Cell line | Type | Treatment | BUB1 | Cleavage | decrease |
| MDA- | TNBC | Compound 1 | ↓ | Yes | 75% |
| MB-231 | | Compound 1 + HCQ | ↓ | Yes | 80% |
| | | Compound 1 + SBI | ↓ | Yes | — |
| | | Comparator 3 | ↓ | Yes | — |
| | | Comparator 1 | ↓ | No | — |
| MDA- | TNBC | Compound 1 | ↓ | Yes | 80% |
| MB-468 | | Compound 1 + HCQ | ↓ | Yes | 80% |
| | | Compound 1 + SBI | ↓ | Yes | 90% |
| | | Comparator 3 | — | No | 50% |
| | | Comparator 3 + HCQ | ↓ | Yes | 45% |
| | | Comparator 3 + SBI | ↓ | Yes | 80% |
| | | Comparator 1 | — | No | 10% |
| | | Comparator 1 + HCQ | ↓ | No | 20% |
| | | Comparator 1 + SBI | ↓ | Yes | 75% |
| HCC-1937 | TNBC | Compound 1 | — | No | 25% |
| | | Compound 1 + HCQ | — | No | 40% |
| | | Compound 1 + SBI | — | No | 50% |
| | | Comparator 3 | — | No | 10% |
| | | Comparator 3 + HCQ | — | No | 25% |
| | | Comparator 3 + SBI | — | No | 30% |
| | | Comparator 1 | — | No | 15% |
| | | Comparator 1 + HCQ | — | No | 25% |
| | | Comparator 1 + SBI | — | No | 30% |

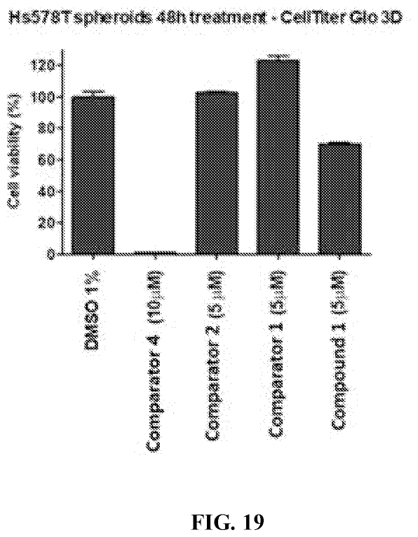

The effect of Compound 1 treatment on breast cancer spheroids was assessed and compared to that of Comparator 1, Comparator 2, and Comparator 4. Hs578T spheroids were grown and treated with Compound 1 (5 μM), Comparator 1 (5 μM), Comparator 2 (5 μM), or Comparator 4 (10 μM) for 48 hours and cell viability assessed. FIG. 18 shows representative images of Hs578T spheroids stained with Hoechst dye and stained for F-actin. Treatment with Compound 1 resulted in a small decrease in spheroid size compared to DMSO, Comparator 1, and Comparator 2. Viability of the spheroids was assessed using CellTiter Glo 3D. FIG. 19 shows the calculated cell viability of the organoids. Treatment with Comparator 4 resulted in significant cell death compared to Compound 1 and Comparators 1 and 2. Treatment with Compound 1 resulted in an ˜40% decrease in organoid viability compared to DMSO, while treatment with Comparator 1 and Comparator 2 had no effect on the organoid viability.

Overall, the data above suggest that Compound 1 has differential effects on inducing cell death and decreasing cell viability compared to the Comparators. This effect appeared to be increased in combination with the autophagy inhibitors HCQ and SBI.

### Example 12: Analysis of Compound 1 Treatment on Cell Death Pathway Activity in Breast Cancer Cells

A series of experiments was designed to determine the effect of Compound 1 treatment on cell death pathway activity in MDA-MB-231 cells.

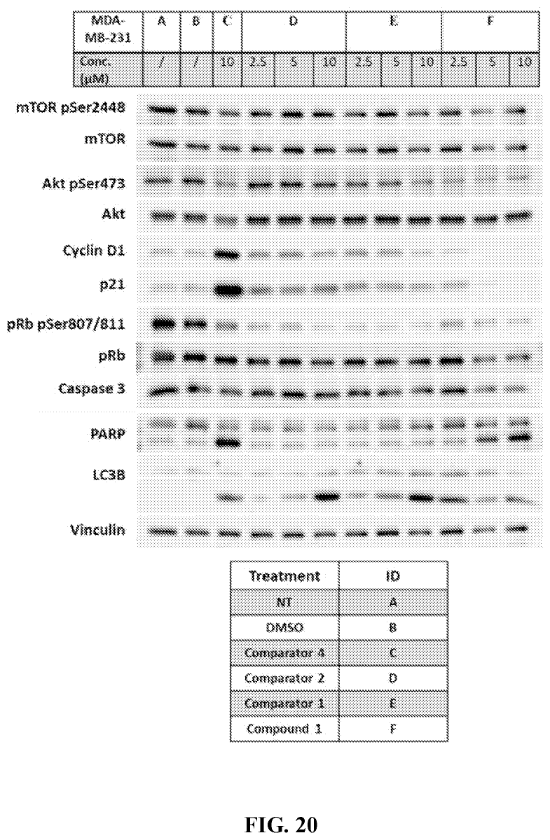

To determine the effect of Compound 1 treatment on protein levels, MDA-MB-231 cells were treated with Comparator 4 (10 μM), Comparator 2 (2.5 μM, 5 μM, and 10 μM), Comparator 1 (2.5 μM, 5 μM, and 10 μM), and Compound 1 (2.5 μM, 5 μM, and 10 μM) for 24 hours and protein expression measured by western blot. FIG. 20 shows the resulting western blot and levels of: mTOR pSer2448, mTOR, Akt pSer473, Akt, Cyclin D1, p21, pRb pSer807/811, pRb, Caspase 3, PARP, LC3B, and Vinculin levels. Compound 1 has differential effects on protein levels compared to the comparators, with Compound 1 treatment decreasing Akt pSer473, Cyclin D1, p21, pRb, and Caspase 3 levels to a greater extent than did Comparator 1, Comparator 2, and Comparator 4 at the concentrations assessed.

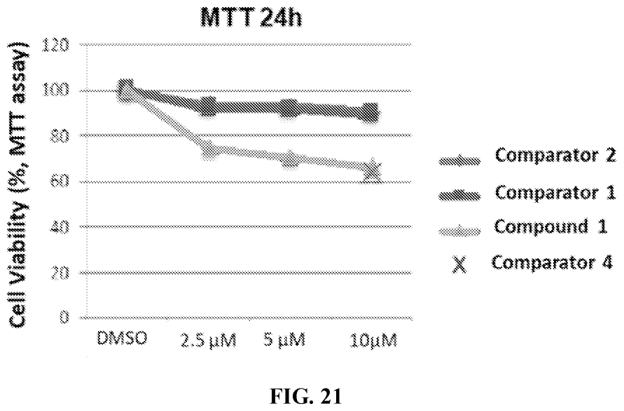

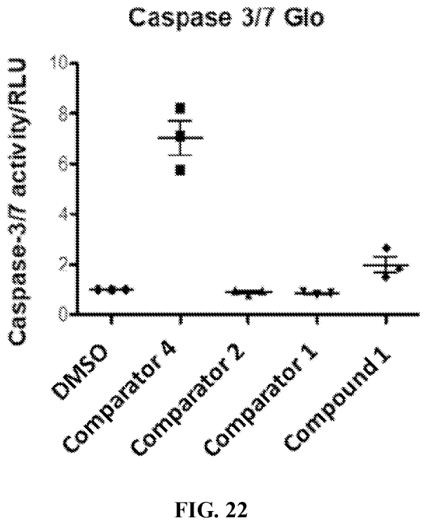

The effect of each of Compound 1, Comparator 1, Comparator 2, and Comparator 4 on MDA-MB-231 cell viability was assessed following 24 hours of treatment using the MTT assay. FIG. 21 shows that compared to both Comparator 1 and Comparator 2, Compound 1 decreases cell viability to a greater extent at 2.5 μM, 5 μM, and 10 μM. 10 μM treatment with Compound 1 resulted in a similar decrease in cell viability as did 10 μM Comparator 4. To determine how Compound 1 induces cell death, Caspase 3/7 activity was measured using the Caspase 3/7 Glo assay. FIG. 22 shows the effect of Compound 1 on Caspase 3/7 activity compared to Comparators 1, 2, and 4. Comparator 4 treatment resulted in the greatest increase in Caspase 3/7 activity among the treatments analyzed. Compound 1 treatment resulted in a modest increase in Caspase 3/7 activity, while Comparator 1 treatment and Comparator 2 treatment each had no effect on Caspase 3/7 activity.

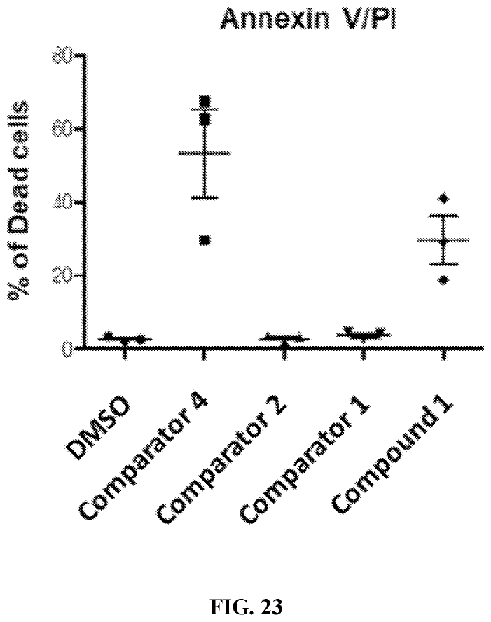

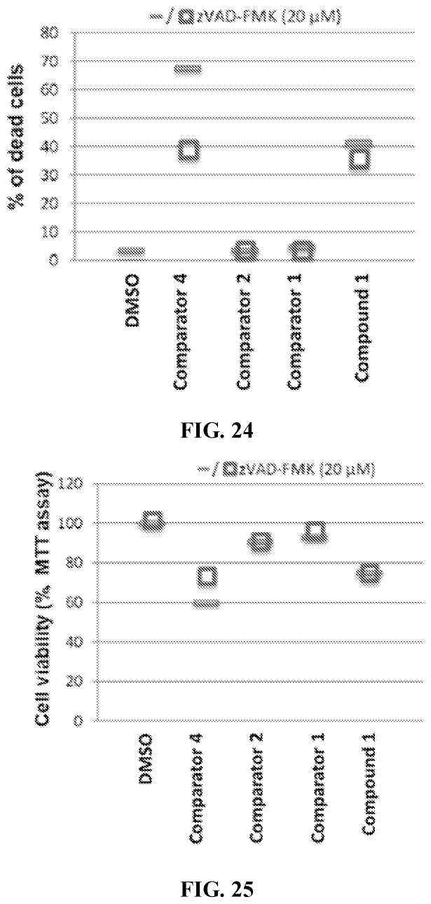

To determine the effect of the different treatment conditions on cell death, Annexin V/PI staining was performed. FIG. 23 shows the effect of Compound 1 treatment on Annexin V/PI staining. Treatment with Compound 1 resulted in ˜40% cell death after 24 hours of treatment. Comparator 4 treatment resulted in ˜55% cell death after 24 hours of treatment, while neither Comparator 1 nor Comparator 2 treatment induced cell death after 24 hours of treatment at the concentrations assessed. To determine the relative contribution of apoptosis to the cell death induced by Compound 1, cells were treated for 24 hours with Compound 1 or Comparator 1, 2, or 4 in the presence or absence of the apoptosis inhibitor zVAD-FMK (20 μM). FIG. 24 shows the effects of zVAD-FMK on cell death as assessed by Annexin V/PI staining, while FIG. 25 shows the effects of zVAD-FMK on cell death as assessed by the MTT assay. With both Annexin V/PI staining and in the MTT assay, treatment with Compound 1 resulted in cell death that was unaffected by zVAD-FMK treatment. Cell death induced by Comparator 1 or Comparator 2 was similarly unaffected by zVAD-FMK treatment. However, Comparator 4 treatment resulted in increased cell death that was partially blocked with zVAD-FMK cotreatment.

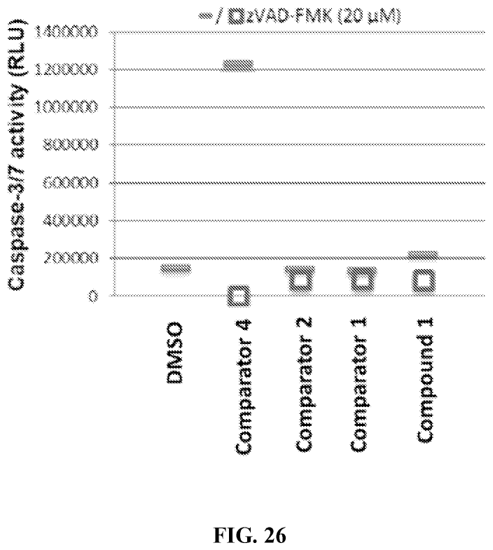

The effect of zVAD-FMK treatment on Caspase 3/7 activity was assessed. FIG. 26 shows the effect of zVAD-FMK treatment on Caspase 3/7 activity induced by Compound 1, Comparator 1, Comparator 2, or Comparator 4. Compound 1 treatment resulted in a small increase in Caspase 3/7 activity that was inhibited by zVAD-FMK treatment. Caspase 3/7 activity was not increased by treatment with Comparator 1 or Comparator 2. Comparator 4 treatment resulted in a large increase in Caspase 3/7 activity that was completely inhibited by zVAD-FMK treatment.

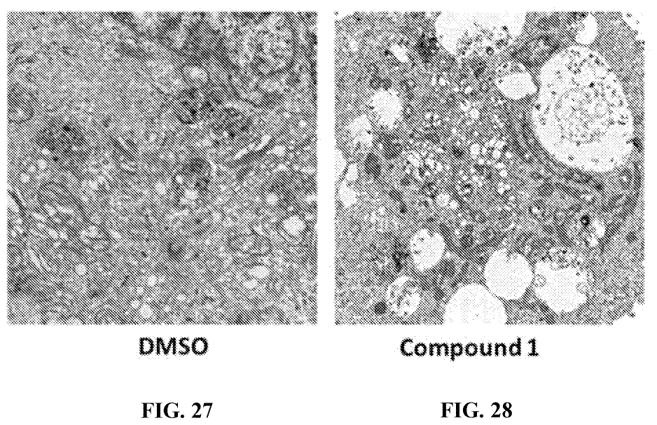

The effect of Compound 1 (5 μM) treatment for 24 hours on cellular morphology was assessed using transmission electron microscopy (TEM) and compared to DMSO. FIG. 27 and FIG. 28 show representative TEM images of MDA-MB-231 cells treated with DMSO or Compound 1. Compound 1 treatment resulted in extensive vacuolization of the cell compared to DMSO.

Overall, the data suggest that Compound 1 treatment results in cell death by a pathway different than those modulated by Comparator 1, Comparator 2, and Comparator 4. Cell death induced by Compound 1 was observed to have a smaller apoptotic contribution than was the cell death induced by Comparator 4, as indicated by the reduced Caspase 3/7 activity and the minimal effects of apoptosis inhibition on the cell death induced by Compound 1.

### Example 13: Effect of Autophagy Induction of Compound 1-Induced Apoptosis in MDA-MB-231 Cells

EXAMPLE 11 indicated that Compound 1 treatment results in vacuolization of the cell and cell death with an apoptotic component. A series of experiments was designed to assess the effect of autophagy on the apoptotic cell death induced by Compound 1 treatment.

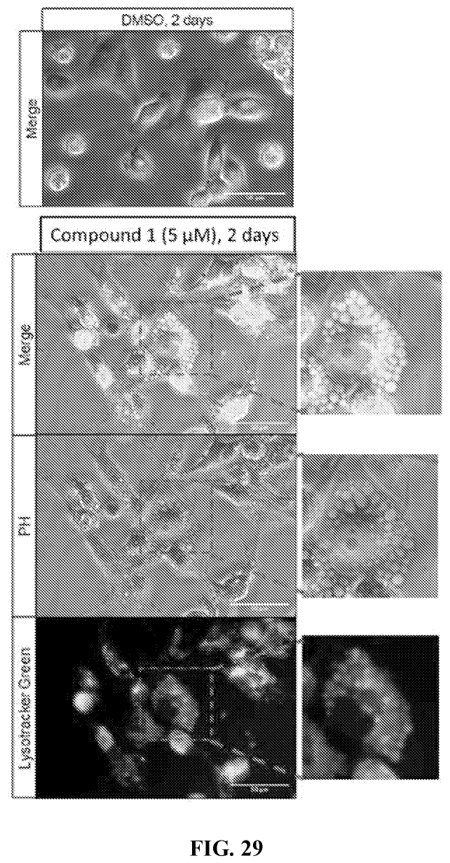

MDA-MB-231 cells were treated with Compound 1 (5 μM) for 2 hours and stained with Lysotracker Green and imaged using phase contrast microscopy to examine the effect of Compound 1 treatment on vacuolization. FIG. 29 shows phase contrast images of MDA-MB-231 cells with Lysotracker Green staining. Compound 1 treatment resulted in extensive cytoplasmic vacuolization that colocalized with Lysotracker green staining. This result suggested that Compound 1 treatment resulted in increased autophagy activity.

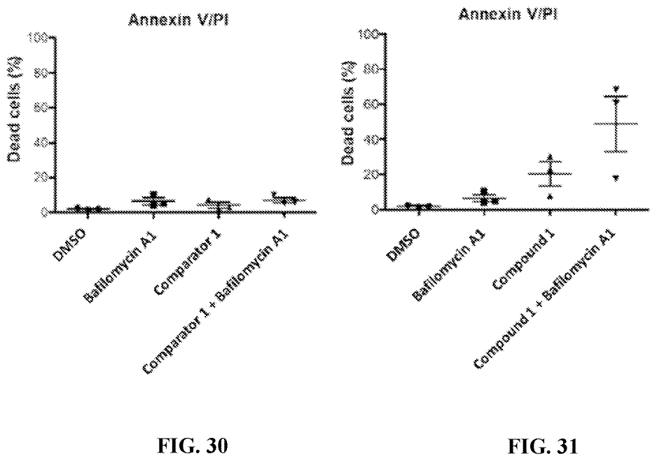

To determine the effect of autophagy on Compound 1-mediated cell death, MDA-MB-231 cells were treated with Compound 1 alone and in combination with the autophagy inhibitor, bafilomycin A1, and compared to Comparator 1 alone and in combination with bafilomycin A1. FIGS. 30 and 31 show cell death induced by Compound 1 and Comparator 1, respectively, as measured by Annexin V/PI staining. Comparator 1 did not induce cell death and bafilomycin A1 cotreatment did not increase death in Comparator 1 treated cells. Compound 1 treatment induced cell death. The effect was increased with bafilomycin A1 cotreatment.

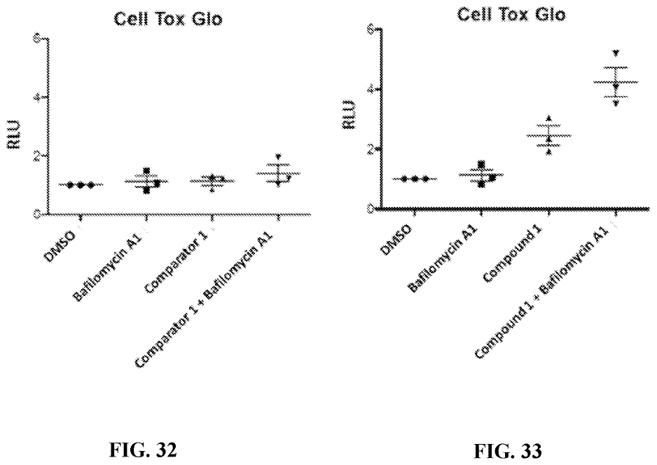

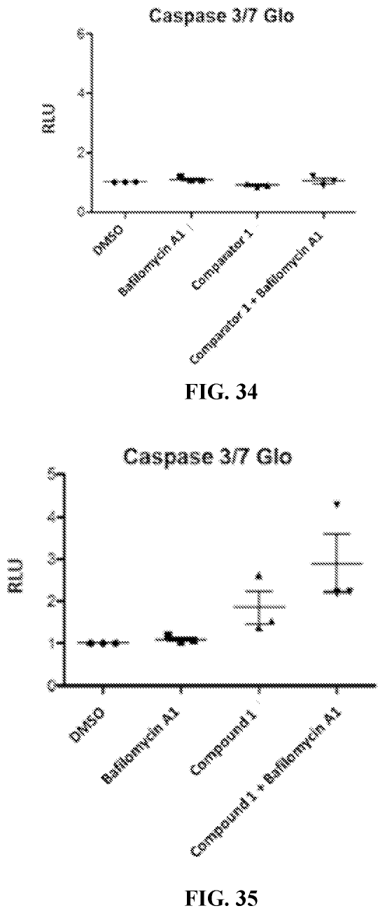

FIGS. 32 and 33 show cell death induced by Compound 1 and Comparator 1 alone or in combination with bafilomycin as measured by Cell Tox Glo cytotoxicity assay. Comparator 1 treatment did not increase cell death. Bafilomycin A1 cotreatment slightly increased cell death induced by Comparator 1. Compound 1 treatment induced cell death that was enhanced by bafilomycin A1 cotreatment. FIGS. 34 and 35 show Caspase 3/7 activity as measured by the Caspase 3/7 Glo assay. Comparator 1 treatment had no effect on Caspase 3/7 activity. Cotreatment with bafilomycin A1 did not increase Caspase 3/7 activity in Comparator 1-treated cells. Compound 1 treatment resulted in increased Caspase 3/7 activity. The effect was enhanced by co-treatment with bafilomycin A1 cotreatment.

Overall, the data above suggest that Compound 1 treatment resulted in increased autophagy and that inhibition of autophagy in Compound 1-treated cells can enhance apoptotic cell death.

### Example 14: Effect of Compound 1 Treatment on PYMT Mouse Breast Cancer Cells

A series of experiments was designed to assess the effect of Compound 1 treatment in mouse mammary tumor virus-polyoma middle tumor-antigen (MMTV-PyMT) cells.

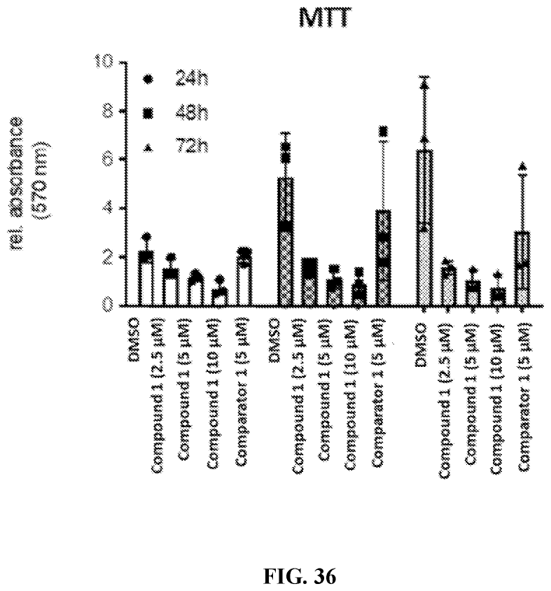

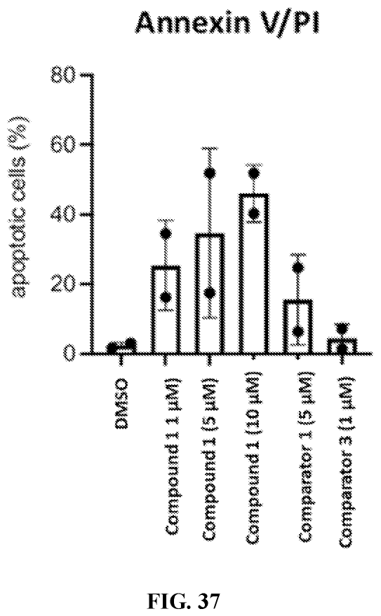

To determine the effect of Compound 1 treatment on MMTV-PyMT cell viability, PyMT cells were treated with increasing concentrations of Compound 1 and 5 μM Comparator 1. FIG. 36 shows MTT cell viability results of PyMT cells treated with Compound 1 and Comparator 1 for 24, 48, and 72 hours. Treatment with increasing concentrations of Compound 1 resulted in increased cell death relative to DMSO for all time-points assessed. Comparator 1 treatment resulted in cell death at 48 and 72 hours that was less than cell death induced by Compound 1 at all concentrations assessed.

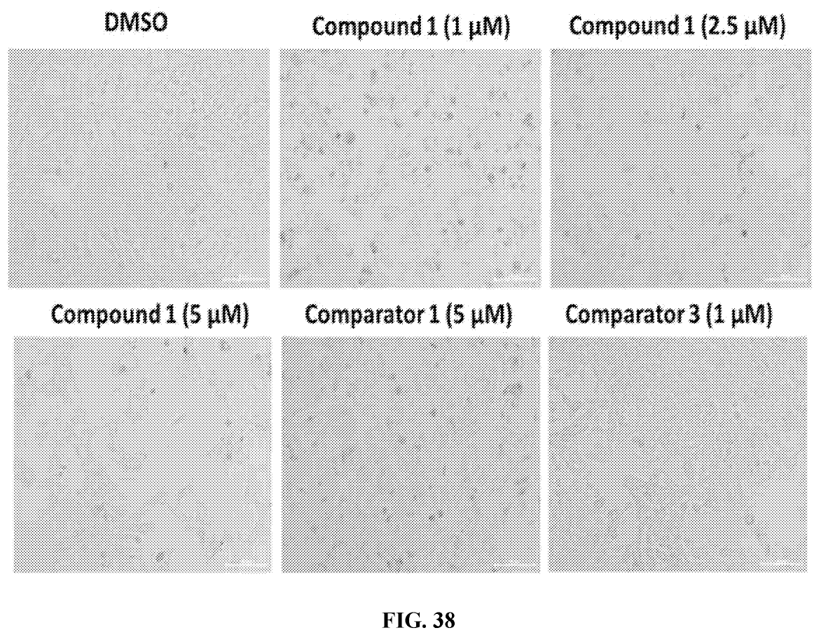

The effect of Compound 1 treatment on the number of apoptotic cells was measured by Annexin V/PI staining and compared to Comparator 1 (5 μM) and Comparator 3 (1 μM). FIG. 37 shows the result of Annexin V/PI staining following 48 hours of treatment with Compound 1, Comparator 1, and Comparator 3. Compound 1 treatment resulted in increased numbers of apoptotic cells that was greater in extent than those of both Comparator 1 and Comparator 3 treatment. The effect of Compound 1, Comparator 1, and Comparator 3 treatment on cellular senescence was assessed using phase contrast imagery. Following 48 hours of treatment, cells were stained for the senescence marker β-galactosidase. FIG. 38 shows representative phase contrast images of cells treated with Compound 1, Comparator 1, or Comparator 3. Compound 1 increased β-galactosidase staining compared to DMSO, while Comparator 1, and to a lesser extent Comparator 3, treatment had no large effect on β-galactosidase staining, indicating that Compound 1 induces senescence.

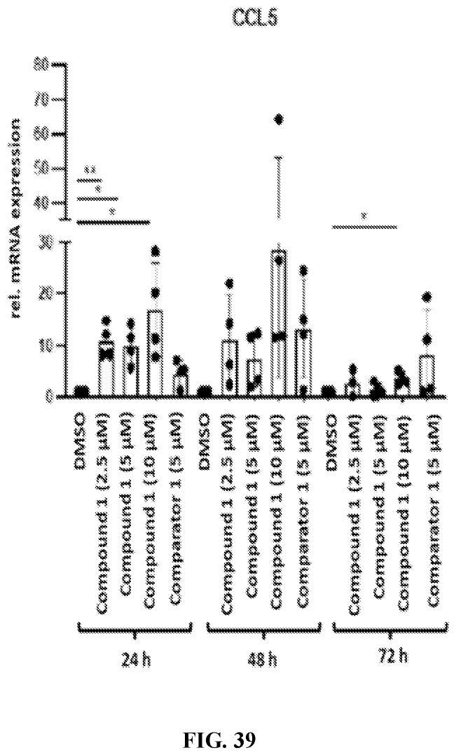

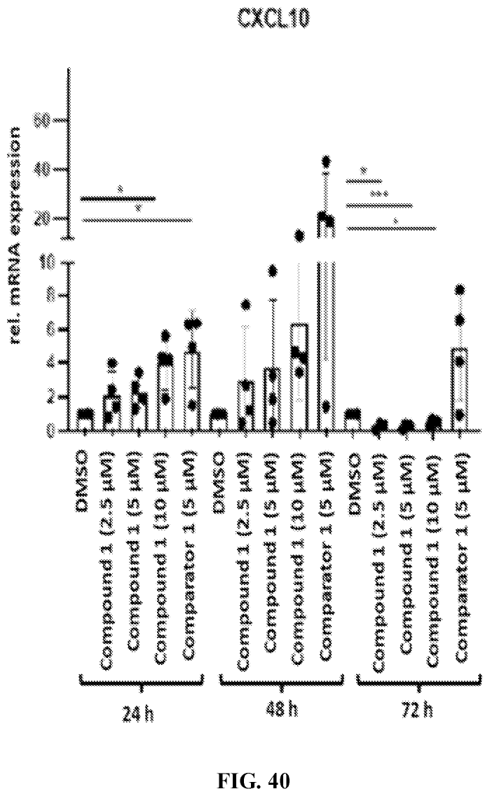

Quantitative polymerase chain reaction (qPCR) was used to assess the effects of Compound 1 treatment and Comparator 1 treatment on T-cell recruiting chemokine levels following 24, 48, and 72 hours of treatment. FIG. 39 shows relative mRNA expression of CCL5. FIG. 40 shows relative mRNA expression of CXCL10. For both chemokines, Compound 1 treatment resulted in a large increase in CCL5 and CXCL10 mRNA levels that were observed to peak at ˜48 hours and then decrease thereafter. CCL5 mRNA levels at 24 and 48 hours following Compound 1 treatment were higher than CCL5 mRNA levels were following Comparator 1. CXCL10 mRNA levels following Comparator 1 treatment were higher than levels were following Compound 1 treatment for all time-points assessed.

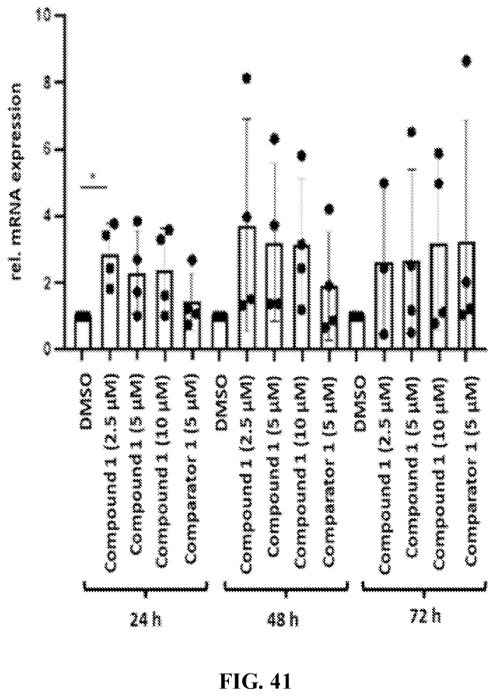

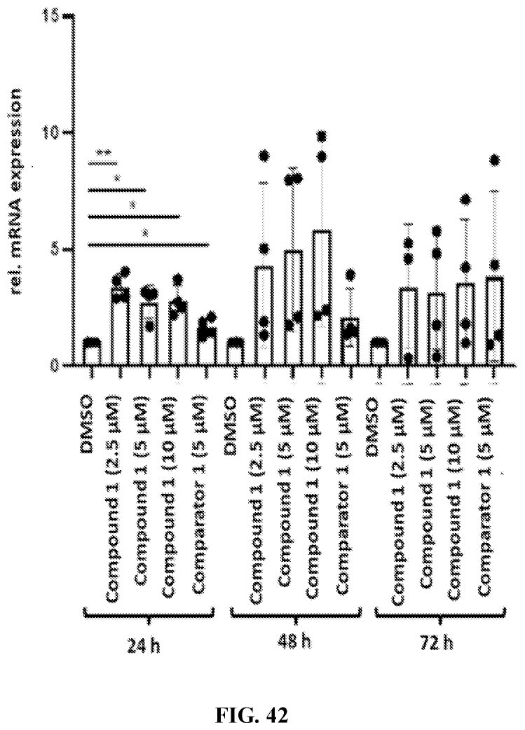

FIG. 41 shows relative mRNA expression of H2D1. FIG. 42 shows relative mRNA expression of B2M. At 24 and 48 hours, Compound 1 treatment increased H2D1 and B2M mRNA levels to a greater extent than Comparator 1 did. At 72 hours, H2D1 mRNA levels were comparable for Compound 1- and Comparator 1-treated cells.

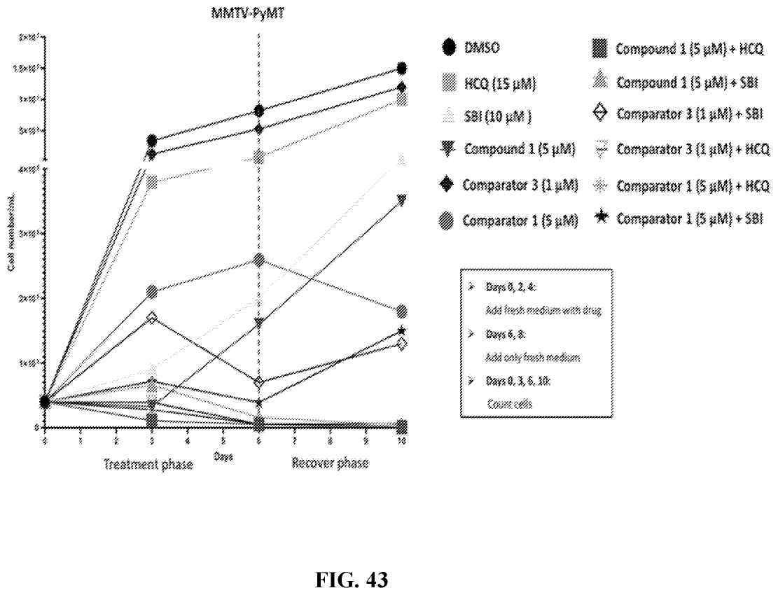

To determine the effects of prolonged treatment with Compound 1, PyMT cells were treated with Compound 1, Comparator 3, or Comparator 1 alone or in the presence of HCQ and SBI for 6 days. Following 6 days of treatment, the drug treatments were stopped and the cells allowed to recover for 4 days. Cell numbers were counted on days 0, 3, 6, and 10, and the results plotted. FIG. 43 shows the results of this experiment, showing the effect of the 6-day treatment phase and the recovery phase of PyMT cell numbers. Compound 1 treatment resulted in a small decrease in cell number at day 3 that then increased throughout the rest of the experiment. Cotreatment with the autophagy inhibitors HCQ and SBI resulted in cell death that persisted after removal of the drugs. Comparator 3 treatment alone resulted in increased cell proliferation. Cotreatment with SBI and Comparator 3 resulted in decreased cell proliferation related to Comparator 3 treatment alone, while cotreatment with HCQ and Comparator 3 resulted in cell death that persisted throughout the duration of the study. Similarly, treatment with Comparator 1 alone resulted in cell proliferation, while cotreatment with SBI resulted in decreased proliferation relative to Comparator 1 alone. Cotreatment with HCQ resulted in cell death that persisted through the duration of the study. This result showed that SBI treatment sensitizes cells only to Compound 1.

The above data suggest that Compound 1 has an activity profile in PyMT cells that is different from those of comparators. Compound 1 treatment induces cell death and T-cell recruiting chemokines in temporal patterns distinct from those of comparators. Compound 1 treatment induces senescence in PyMT cells and SBI treatment sensitizes cells only to Compound 1 and not to comparators.

### Example 15: Compound I is a Potent Inhibitor of Different Targets

This example is directed to testing the inhibitory effect of Compound 1 on different targets.

### NanoBRET Assay:

HEK293 cells were transfected with 1 ug of NanoLuc-CDK16 Fusion vector and 9 ug of either carrier DNA or cyclin Y DAN. The HEK293 cells transiently expressing the vectors were then seeded into the wells of 384-well plates. The cells were pre-treated with the NanoBRET Tracer K-10 (0.5 uM) and then treated with 3-fold serial dilutions (10 uM-0.5 nM), of Compound 1 or the reference compound APY-69 for 1 hour. The BRET signal was measured on an Envision2104 Multilabel Reader. IC50 value was calculated and IC50 curve was plotted using the GraphPad Prism 4 program based on a sigmoidal dose response equation.

### HotSpot Assay:

Reagents: Base reaction buffer: 20 mM Hepes (pH 7.5), 10 mM MgCl2, 1 mM EGTA, 0.01% Brij35, 0.02 mg/ml BSA, 0.1 mM Na3VO4, 2 mM DTT, 1% DMSO. Required cofactors were added individually to each kinase reaction (Compound 1, Comparator 1, Comparator 2, and Comparator 3). Substrate was prepared in freshly prepared base reaction buffer. Any required cofactors were delivered to the substrate solution above. Indicated kinase was delivered into the substrate solution and the solution was gently mixed. Compounds were delivered in 100% DMSO into the kinase reaction mixture by Acoustic technology (Echo550; nanoliter range) and incubated for 20 minutes at room temperature. 33P-ATP was delivered into the reaction mixture to initiate the reaction. The kinase reaction was incubated for 2 hours at room temperature. Kinase activity was detected by P81 filter-binding method.

### Kinase Assay:

Kinase-tagged T7 phage strains were prepared in an E. coli host derived from the BL21 strain. E. coli were grown to log-phase and infected with T7 phage and incubated with shaking at 32° C. until lysis. The lysates were spun by centrifuge and filtered to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific binding. Binding reactions were assembled by combining kinases, liganded affinity beads, and test compounds in 1× binding buffer (20% SeaBlock, 0.17×PBS, 0.05% Tween 20, 6 mM DTT). Test compounds were prepared as 111× stocks in 100% DMSO. Kds were determined using an 11-point 3-fold compound dilution series with three DMSO control points.

All compounds (Compound 1) for Kd measurements were distributed by acoustic transfer (non-contact dispensing) in 100% DMSO. The compounds were then diluted directly into the assays such that the final concentration of DMSO was 0.9%. All reactions performed in polypropylene 384-well plate. Each was a final volume of 0.02 ml. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μM non-biotinylated affinity ligand) and incubated at room temperature with shaking for 30 minutes. The kinase concentration in the eluates was measured by qPCR. Binding constants (Kds) were calculated with a standard dose-response curve using the Hill equation: Response=Background+Signal−Background 1+(KdHill Slope/DoseHill Slope) The Hill Slope was set to −1. Curves were fitted using a non-linear least square fit with the Levenberg-Marquardt algorithm.

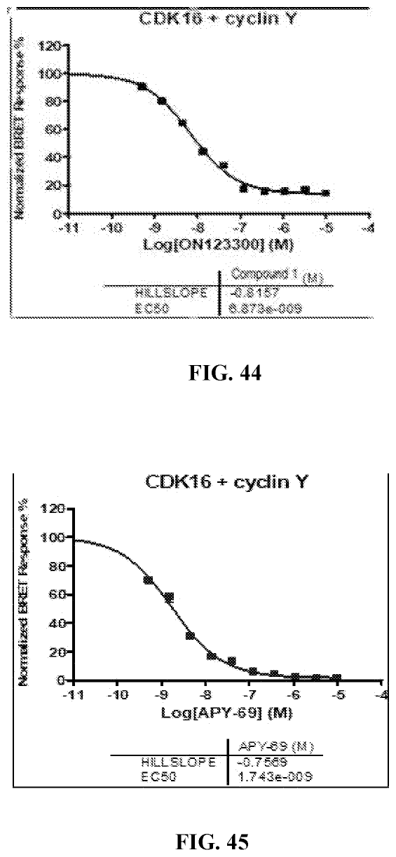

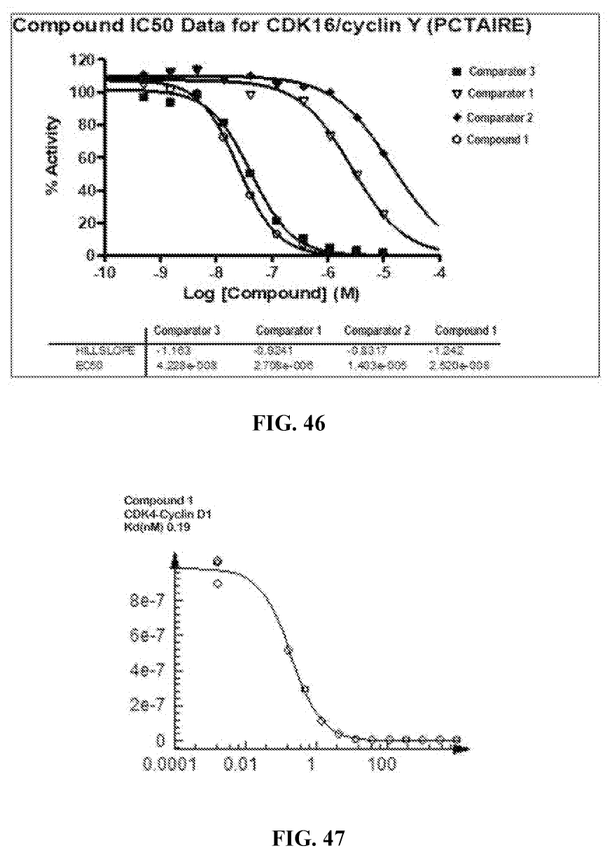

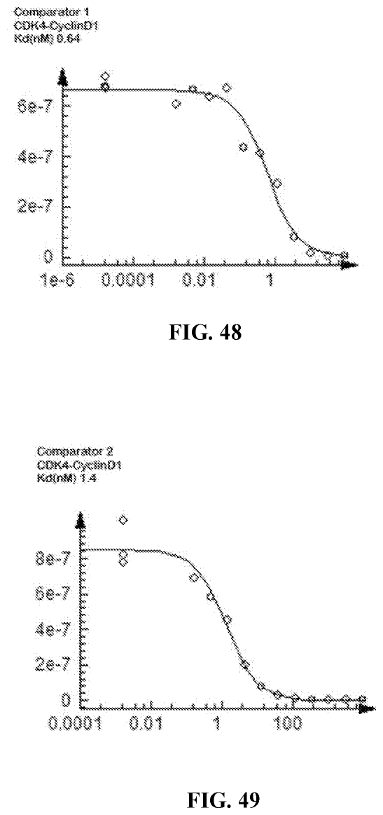

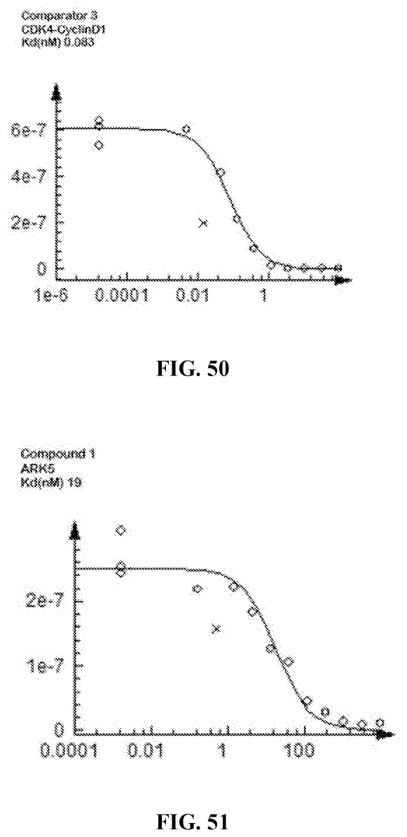

The results are shown in Tables 8-10 and FIGS. 44-51. FIG. 44 shows the IC50 curve of Compound 1 as determined by Cellular NanoBret Assay. FIG. 45 shows the IC50 curve of Reference compound APY-69 (29B) as determined by Cellular NanoBret Assay. FIG. 46 shows comparison of IC50 data of Compound 1, Comparator 1, Comparator 2, and Comparator 3 for CDK16/Cyclin Y as calculated by HotSpot™ Kinase Assay. FIG. 47 shows curve images obtained from Kinase assays for Compound 1 for CDk4-CyclinD1. The amount of kinase measured by qPCR (Signal; y-axis) was plotted against the corresponding compound concentration in nM in log 10 scale (x-axis). Data points marked with an “x” were not used for Kd determination. FIG. 48 shows curve images obtained from Kinase assays for Comparator 1 for CDk4-CyclinD1. The amount of kinase measured by qPCR (Signal; y-axis) was plotted against the corresponding compound concentration in nM in log 10 scale (x-axis). Data points marked with an “x” were not used for Kd determination. FIG. 49 shows curve images obtained from Kinase assays for Comparator 2 for CDk4-CyclinD1. The amount of kinase measured by qPCR (Signal; y-axis) was plotted against the corresponding compound concentration in nM in log 10 scale (x-axis). Data points marked with an “x” were not used for Kd determination. FIG. 50 shows curve images obtained from Kinase assays for Compound 1 for Compound 1 for Ark5. The amount of kinase measured by qPCR (Signal; y-axis) was plotted against the corresponding compound concentration in nM in log 10 scale (x-axis). Data points marked with an “x” were not used for Kd determination. FIG. 51 shows curve images obtained from Kinase assays for Compound 1 for Ark5. The amount of kinase measured by qPCR (Signal; y-axis) was plotted against the corresponding compound concentration in nM in log 10 scale (x-axis). Data points marked with an “x” were not used for Kd determination.

Results show that Compound 1 is a potent inhibitor with nanomolar Kd values against CD4/cyclin D1. Compound 1 is a potent kinase inhibitor with nanomolar Kd values against NUAK 1.

TABLE 8
Compound 1 is a potent kinase inhibitor with nanomolar
Kd values against NUAK1

| | NUAK1 | NUAK1 | NUAK1 | |
|---|---|---|---|---|
| | In Vitro | NanoBret | In Vitro | CETSA |
| Compound 1 | 5 | 42 | 19 | NEG |

TABLE 9
Compound 1 is a potent inhibitor with nanomolar Kd values
against CD4/cyclin D1

| | IC50 (nM) | | Kd (nM) | |
|---|---|---|---|---|
| | CDK4/cyclin | CDK4/cyclin | CDK4/cyclin | |
| | D1 | D1 | D1 | |
| Compound | In Vitro | NanoBret | In Vitro | CETSA |
| Compound 1 | 2.0 | 1.0 | 0.18 | POS |
| Comparator 3 | 0.8 | 0.8 | 0.08 | ND |
| Comparator 1 | 2.0 | ND | 0.75 | POS |
| Comparator 2 | 3.0 | ND | 1.3 | ND |

TABLE 10
Comparison of raw data ratio of Compound 1 and
reference compound APY-69 at 600 nm/460 nm

| | | | Compound 1 | | APY-69 | |
|---|---|---|---|---|---|---|
| Conc. (μM) | Conc. (M) | Log10 (M) | Data 1 | Data 2 | Data 1 | Data 2 |
| 10.0000 | 1.00E−05 | −5.00 | 0.02733113 | 0.028313157 | 0.019692099 | 0.020322028 |
| 3.3333 | 3.33E−06 | −5.48 | 0.029961181 | 0.029081261 | 0.019344185 | 0.020322591 |
| 1.1111 | 1.11E−06 | −5.95 | 0.028358997 | 0.028993845 | 0.01993942 | 0.020630997 |
| 0.3704 | 3.70E−07 | −6.43 | 0.028372276 | 0.029418654 | 0.022005858 | 0.021780768 |
| 0.1235 | 1.23E−07 | −6.91 | 0.029631125 | 0.030048434 | 0.022515473 | 0.023021775 |
| 0.0412 | 4.12E−08 | −7.39 | 0.040360411 | 0.040478788 | 0.026873636 | 0.027914372 |
| 0.0137 | 1.37E−08 | −7.86 | 0.046263485 | 0.046829832 | 0.028987492 | 0.030014845 |
| 0.0046 | 4.57E−09 | −8.34 | 0.058828464 | 0.059842491 | 0.037698321 | 0.039252129 |
| 0.0015 | 1.52E−09 | −8.82 | 0.069082071 | 0.069193035 | 0.053675757 | 0.057637521 |
| 0.0005 | 5.08E−10 | −9.29 | 0.075573696 | 0.076100228 | 0.061511424 | 0.064370937 |
| DMSO | DMSO | DMSO | 0.081794223 | 0.081686793 | 0.082701009 | 0.081078979 |

| | | | | |
|---|---|---|---|---|
| Background | 0.019054726 | 0.019008031 | 0.019002798 | 0.019145368 |

| | | |
|---|---|---|
| Average of background | 0.01903 | 0.01907 |

What is claimed is:

1. A method of treating breast cancer in a subject in need thereof, the method comprising administering to the subject a therapeutically-effective amount of a compound of formula (I) 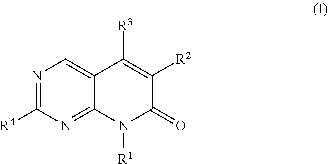
wherein:
R¹ is alkyl, alkenyl, alkynyl, cycloalkyl, aryl, heteroaryl or heterocyclyl, each of which is unsubstituted or substituted, or hydrogen;
R² is alkyl, alkenyl, alkynyl, cycloalkyl, aryl, heteroaryl, heterocyclyl, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
R³ is alkyl, alkenyl, alkynyl, cycloalkyl, aryl, heterocyclyl or heteroaryl, each of which is unsubstituted or substituted, or —CN, hydrogen, or halogen;
R⁴ is alkyl, alkenyl, alkynyl, cycloalkyl, aryl, heterocyclyl, heteroaryl, C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
each R⁵ and R⁶ is independently alkyl, alkenyl, alkynyl, cycloalkyl, aryl, heterocyclyl, or heteroaryl, each of which is independently substituted or unsubstituted, or —CN, hydrogen, or halogen;
provided that, when R⁴ is —NR⁵R⁶ and one of R⁵ and R⁶ is pyridyl, then R² is not —C(O)CH₃,
or a pharmaceutically-acceptable salt thereof,
wherein the administering is once daily.

2. The method of claim 1, wherein R¹ is cycloalkyl.

3. The method of claim 1, wherein R¹ is cyclopentyl.

4. The method of claim 1, wherein R² is CN.

5. The method of claim 1, wherein R³ is hydrogen.

6. The method of claim 1, wherein R⁴ is —NR⁵R⁶.

7. The method of claim 6, wherein one of R⁵ and R⁶ is hydrogen.

8. The method of claim 6, wherein one of R⁵ and R⁶ is phenyl.

9. The method of claim 6, wherein one of R⁵ and R⁶ is phenyl substituted with heterocyclyl.

10. The method of claim 6, wherein one of R⁵ and R⁶ is phenyl substituted with piperazinyl.

11. The method of claim 6, wherein one of R⁵ and R⁶ is phenyl substituted with 4-methyl piperazinyl.

12. The method of claim 1, wherein R⁴ is 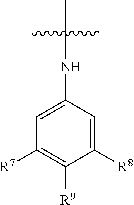
wherein:
R⁷ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen;
R⁸ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen; and
R⁹ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen.

13. The method of claim 12, wherein R⁷ is hydrogen.

14. The method of claim 12, wherein R⁸ is hydrogen.

15. The method of claim 12, wherein R⁹ is unsubstituted or substituted heterocyclyl.

16. The method of claim 12, wherein R⁹ is unsubstituted or substituted piperazinyl.

17. The method of claim 12, wherein R⁹ is 4-methyl piperazinyl.

18. The method of claim 12, wherein the compound is a compound of formula (II)

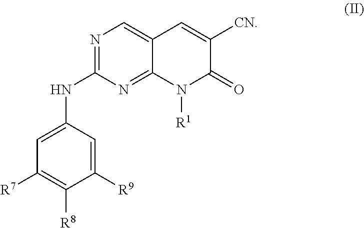

19. The method of claim 12, wherein the compound is a compound of formula (III) 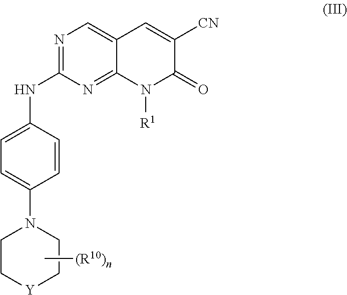
wherein:
Y is O, S, or NR¹¹;
each R¹⁰ is independently alkyl, alkenyl, alkynyl, cycloalkyl, aryl, heterocyclyl, heteroaryl, —OR⁵, —SR⁵, or —NR⁵R⁶, each of which is unsubstituted or substituted;
R¹¹ is alkyl, alkenyl, alkynyl, cycloalkyl, aryl, heterocyclyl, or heteroaryl, each of which is independently substituted or unsubstituted, or hydrogen or halogen; and
n is 0, 1, 2, 3, 4, 5, 6, 7, or 8.

20. The method of claim 19, wherein R¹ is cycloalkyl.

21. The method of claim 19, wherein R¹ is cyclopentyl.

22. The method of claim 19, wherein Y is NR¹¹.

23. The method of claim 22, wherein R¹¹ is alkyl.

24. The method of claim 22, wherein R¹¹ is methyl.

25. The method of claim 19, wherein n is 0.

26. The method of claim 1, wherein the compound is 8-cyclopentyl-2-((4-(4-methylpiperazin-1-yl)phenyl)amino)-7-oxo-7,8-dihydropyrido[2,3-d]pyrimidine-6-carbonitrile, or a pharmaceutically-acceptable salt thereof.

27. The method of claim 26, wherein the compound is the pharmaceutically-acceptable salt and the pharmaceutically-acceptable salt is monolactate.

28. The method of claim 1, wherein the therapeutically-effective amount is about 80 mg to about 500 mg per day.

29. The method of claim 1, wherein the breast cancer is a hormone receptor positive breast cancer.

30. The method of claim 1, wherein the breast cancer is an estrogen receptor positive breast cancer.

31. The method of claim 1, wherein the breast cancer is a progesterone receptor positive breast cancer.

32. The method of claim 1, wherein the breast cancer is an estrogen receptor positive, progesterone receptor positive breast cancer.

33. The method of claim 1, wherein the breast cancer is a human epidermal growth factor receptor 2 (HER2) negative breast cancer.

34. The method of claim 1, wherein the breast cancer is a hormone receptor positive, human epidermal growth factor receptor 2 (HER2) negative breast cancer.

35. The method of claim 1, wherein the breast cancer is an estrogen receptor positive, progesterone receptor positive, human epidermal growth factor receptor 2 (HER2) negative breast cancer.

36. The method of claim 1, wherein the breast cancer is a hormone receptor negative breast cancer.

37. The method of claim 1, wherein the breast cancer is an estrogen receptor negative breast cancer.

38. The method of claim 1, wherein the breast cancer is a progesterone receptor negative breast cancer.

39. The method of claim 1, wherein breast cancer is a hormone receptor negative, human epidermal growth factor receptor 2 (HER2) negative breast cancer.

40. The method of claim 1, wherein breast cancer is an estrogen receptor negative, progesterone receptor negative, human epidermal growth factor receptor 2 (HER2) negative breast cancer.

41. The method of claim 40, wherein the breast cancer expresses Mitotic checkpoint serine/threonine-protein kinase BUB1.

42. The method of claim 1, wherein the breast cancer is advanced breast cancer.

43. The method of claim 1, wherein the breast cancer is metastatic breast cancer.

44. The method of claim 1, wherein the breast cancer is a hormone receptor positive, human epidermal growth factor receptor 2 (HER2) positive metastatic breast cancer.

45. The method of claim 1, wherein the breast cancer is an estrogen receptor positive, progesterone receptor positive, human epidermal growth factor receptor 2 (HER2) positive metastatic breast cancer.

46. The method of claim 1, wherein the breast cancer is hormone receptor negative, human epidermal growth factor receptor 2 (HER2) positive metastatic breast cancer.

47. The method of claim 1, wherein the breast cancer is an estrogen receptor negative, progesterone receptor negative, human epidermal growth factor receptor 2 (HER2) positive metastatic breast cancer.

48. The method of claim 1, wherein the subject is a woman.

49. The method of claim 1, wherein the subject is a postmenopausal woman.

50. The method of claim 1, wherein the subject is a man.

51. The method of claim 1, wherein the subject received a therapy other than the compound for the breast cancer prior to the administering.

52. The method of claim 51, wherein the therapy was received after the subject was diagnosed with the breast cancer.

53. The method of claim 51, wherein the subject has not responded to the therapy.

54. The method of claim 51, wherein the subject experienced a relapse of the breast cancer after the therapy.

55. The method of claim 1, wherein the administering is oral.

56. The method of claim 1, wherein the administering is intravenous.

57. The method of claim 1, wherein the administering is once daily for at least 1 week.

58. The method of claim 1, wherein the administering is once daily for at least 2 weeks.

59. The method of claim 1, wherein the administering is once daily for at least 4 weeks.

60. The method of claim 1, wherein the administering is oral on a 4-week cycle of:
(i) a continuous, three-week period of once-daily administration; and
(ii) immediately following the three-week period, one week of no administration.

61. The method of claim 1, wherein the administering is oral on a 4-week cycle of:
(i) a continuous, three-week period of once-daily, morning administration, wherein the therapeutically-effective amount is from about 80 mg to about 500 mg; and
(ii) immediately following the three-week period, one week of no administration.

62. The method of claim 1, wherein the compound is administered in a pharmaceutical composition, wherein the pharmaceutical composition is in a unit dosage form, the unit dosage form further comprising a pharmaceutically-acceptable excipient.

63. The method of claim 62, wherein the unit dosage form comprises about 40 mg of 8-cyclopentyl-2-((4-(4-methylpiperazin-1-yl)phenyl)amino)-7-oxo-7,8-dihydropyrido[2,3-d]pyrimidine-6-carbonitrile.

64. The method of claim 62, wherein the unit dosage form comprises about 48.4 mg of 8-cyclopentyl-2-((4-(4-methylpiperazin-1-yl)phenyl)amino)-7-oxo-7,8-dihydropyrido[2,3-d]pyrimidine-6-carbonitrile monolactate.

65. The method of claim 62, wherein the unit dosage form is a capsule.

66. The method of claim 62, wherein the unit dosage form is a tablet.

67. The method of claim 1, wherein the administering occurs in a morning of a day.

68. The method of claim 1, wherein the subject is in a fasted state.

69. The method of claim 1, further comprising administering to the subject a therapeutically-effective amount of a second therapeutic agent.

70. The method of claim 69, wherein the second therapeutic agent is an estrogen receptor modulator.

71. The method of claim 69, wherein the second therapeutic agent is an estrogen receptor blocker.

72. The method of claim 69, wherein the second therapeutic agent is an aromatase inhibitor.

73. The method of claim 69, wherein the second therapeutic agent is an aromatase inhibitor, wherein the aromatase inhibitor is administered as an initial endocrine-based therapy.

74. The method of claim 69, wherein the second therapeutic agent is letrozole or a pharmaceutically-acceptable salt thereof.

75. The method of claim 74, wherein the letrozole or pharmaceutically-acceptable salt thereof is administered in a pharmaceutical composition, wherein the pharmaceutical composition is in a unit dosage form, the unit dosage form further comprising a pharmaceutically-acceptable excipient.

76. The method of claim 75, wherein the unit dosage form comprises about 2.5 mg of letrozole.

77. The method of claim 75, wherein the unit dosage form is a tablet.

78. The method of claim 69, wherein the second therapeutic agent is a selective estrogen receptor degrader.

79. The method of claim 78, wherein the selective estrogen receptor degrader is fulvestrant.

80. The method of claim 78, wherein the selective estrogen receptor degrader is fulvestrant, wherein the fulvestrant is administered following an endocrine therapy.

81. The method of claim 69, wherein the second therapeutic agent is a progestin.

82. The method of claim 81, wherein the progestin is megestrol acetate.

83. The method of claim 82, wherein megestrol acetate is administered in a pharmaceutical composition, wherein the pharmaceutical composition is in a unit dosage form, the unit dosage form further comprising a pharmaceutically-acceptable excipient.

84. The method of claim 83, wherein the unit dosage form comprises about 125 mg/mL of megestrol acetate.

85. The method of claim 83, wherein the unit dosage form is an oral suspension.

86. The method of claim 83, wherein the megestrol acetate is administered at a dose of about 625 mg per day.

87. The method of claim 69, wherein the administering of the second therapeutic agent is once daily.

88. The method of claim 69, wherein the second therapeutic agent is a vascular endothelial growth factor inhibitor.

89. The method of claim 69, wherein the second therapeutic agent is a phosphoinositide 3-kinase inhibitor.

90. The method of claim 69, wherein the administering of the second therapeutic agent is oral.

91. The method of claim 69, wherein the administering of the second therapeutic agent is intravenous.

92. The method of claim 69, wherein the administering of the second therapeutic agent is intramuscular.

93. The method of claim 69, wherein the administering of the compound of formula (I) is oral, and the administering of the second therapeutic agent is oral.

94. The method of claim 69, wherein the administering of the compound of formula (I) is oral, and the administering of the second therapeutic agent is intravenous.

95. The method of claim 69, wherein the administering of the compound of formula (I) is oral, and the administering of the second therapeutic agent is intramuscular.

96. The method of claim 69, wherein the administering of the compound of formula (I) is prior to the administering of the second therapeutic agent.

97. The method of claim 69, wherein the administering of the second therapeutic agent is prior to the administering of the compound of formula (I).

98. The method of claim 69, wherein the administering of the compound of formula (I) is concurrently with the administering of the second therapeutic agent.

99. A method of treating breast cancer in a subject in need thereof, the method comprising administering to the subject a therapeutically-effective amount of a compound of formula (I): 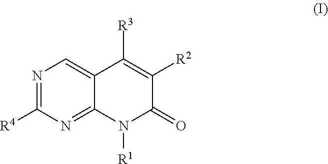
wherein:
R¹ is alkyl, alkenyl, alkynyl, cycloalkyl, aryl, heteroaryl or heterocyclyl, each of which is unsubstituted or substituted, or hydrogen;
R² is alkyl, alkenyl, alkynyl, cycloalkyl, aryl, heteroaryl, heterocyclyl, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
R³ is alkyl, alkenyl, alkynyl, cycloalkyl, aryl, heterocyclyl or heteroaryl, each of which is unsubstituted or substituted, or —CN, hydrogen, or halogen;
R⁴ is alkyl, alkenyl, alkynyl, cycloalkyl, aryl, heterocyclyl, heteroaryl, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
each R⁵ and R⁶ is independently alkyl, alkenyl, alkynyl, cycloalkyl, aryl, heterocyclyl, or heteroaryl, each of which is independently substituted or unsubstituted, or —CN, hydrogen, or halogen;
provided that, when R⁴ is —NR⁵R⁶ and one of R⁵ and R⁶ is pyridyl, then R² is not —C(O)CH₃,
or a pharmaceutically-acceptable salt thereof;
wherein the administering is a 4-week cycle of:
(i) a continuous, three-week period of once-daily administration; and
(ii) immediately following the three-week period, one week of no administration.

100. The method of claim 99, wherein R¹ is cycloalkyl.

101. The method of claim 99, wherein R¹ is cyclopentyl.

102. The method of claim 99, wherein R² is CN.

103. The method of claim 99, wherein R³ is hydrogen.

104. The method of claim 99, wherein R⁴ is —NR⁵R⁶.

105. The method of claim 104, wherein one of R⁵ and R⁶ is hydrogen.

106. The method of claim 104, wherein one of R⁵ and R⁶ is phenyl.

107. The method of claim 104, wherein one of R⁵ and R⁶ is phenyl substituted with heterocyclyl.

108. The method of claim 104, wherein one of R⁵ and R⁶ is phenyl substituted with piperazinyl.

109. The method of claim 104, wherein one of R⁵ and R⁶ is phenyl substituted with 4-methyl piperazinyl.

110. The method of claim 99, wherein R⁴ is 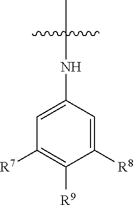
wherein:
R⁷ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen;
R⁸ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen; and
R⁹ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen.

111. The method of claim 110, wherein R⁷ is hydrogen.

112. The method of claim 110, wherein R⁸ is hydrogen.

113. The method of claim 110, wherein R⁹ is unsubstituted or substituted heterocyclyl.

114. The method of claim 110, wherein R⁹ is unsubstituted or substituted piperazinyl.

115. The method of claim 110, wherein R⁹ is 4-methyl piperazinyl.

116. The method of claim 110, wherein the compound is a compound of formula (II)

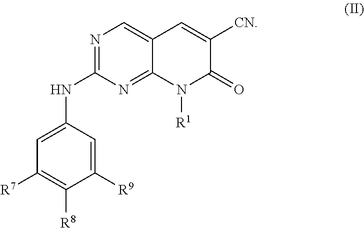

117. The method of claim 110, wherein the compound is a compound of formula (III) 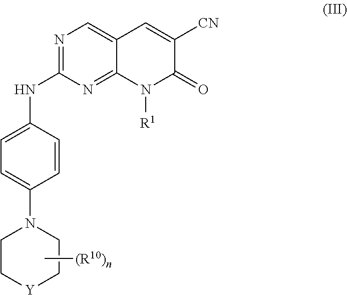
wherein:
Y is O, S, or NR¹¹;
each R¹⁰ is independently alkyl, alkenyl, alkynyl, cycloalkyl, aryl, heterocyclyl, heteroaryl, —OR⁵, —SR⁵, or —NR⁵R⁶, each of which is unsubstituted or substituted;
R¹¹ is alkyl, alkenyl, alkynyl, cycloalkyl, aryl, heterocyclyl, or heteroaryl, each of which is independently substituted or unsubstituted, or hydrogen or halogen; and
n is 0, 1, 2, 3, 4, 5, 6, 7, or 8.

118. The method of claim 117, wherein R¹ is cycloalkyl.

119. The method of claim 117, wherein R¹ is cyclopentyl.

120. The method of claim 117, wherein Y is NR¹¹.

121. The method of claim 120, wherein R¹¹ is alkyl.

122. The method of claim 120, wherein R¹¹ is methyl.

123. The method of claim 117, wherein n is 0.

124. The method of claim 99, wherein the compound is 8-cyclopentyl-2-((4-(4-methylpiperazin-1-yl)phenyl)amino)-7-oxo-7,8-dihydropyrido[2,3-d]pyrimidine-6-carbonitrile, or a pharmaceutically-acceptable salt thereof.

125. The method of claim 124, wherein the compound is the pharmaceutically-acceptable salt and the pharmaceutically-acceptable salt is monolactate.

126. The method of claim 99, wherein the therapeutically-effective amount is about 40 mg to about 500 mg per day.

127. The method of claim 99, wherein the breast cancer is a hormone receptor positive breast cancer.

128. The method of claim 99, wherein the breast cancer is an estrogen receptor positive breast cancer.

129. The method of claim 99, wherein the breast cancer is a progesterone receptor positive breast cancer.

130. The method of claim 99, wherein the breast cancer is an estrogen receptor positive, progesterone receptor positive breast cancer.

131. The method of claim 99, wherein the breast cancer is a human epidermal growth factor receptor 2 (HER2) positive breast cancer.

132. The method of claim 99, wherein the breast cancer is a hormone receptor positive, human epidermal growth factor receptor 2 (HER2) positive breast cancer.

133. The method of claim 99, wherein the breast cancer is an estrogen receptor positive, progesterone receptor positive, human epidermal growth factor receptor 2 (HER2) positive breast cancer.

134. The method of claim 99, wherein the breast cancer is a hormone receptor positive breast cancer.

135. The method of claim 99, wherein the breast cancer is an estrogen receptor positive breast cancer.

136. The method of claim 99, wherein the breast cancer is a progesterone receptor positive breast cancer.

137. The method of claim 99, wherein breast cancer is a hormone receptor negative, human epidermal growth factor receptor 2 (HER2) positive breast cancer.

138. The method of claim 99, wherein breast cancer is an estrogen receptor negative, progesterone receptor negative, human epidermal growth factor receptor 2 (HER2) positive breast cancer.

139. The method of claim 138, wherein the breast cancer expresses Mitotic checkpoint serine/threonine-protein kinase BUB1.

140. The method of claim 99, wherein the breast cancer is advanced breast cancer.

141. The method of claim 99, wherein the breast cancer is metastatic breast cancer.

142. The method of claim 99, wherein the breast cancer is a hormone receptor positive, human epidermal growth factor receptor 2 (HER2) positive metastatic breast cancer.

143. The method of claim 99, wherein the breast cancer is an estrogen receptor positive, progesterone receptor positive, human epidermal growth factor receptor 2 (HER2) positive metastatic breast cancer.

144. The method of claim 99, wherein the breast cancer is hormone receptor negative, human epidermal growth factor receptor 2 (HER2) positive metastatic breast cancer.

145. The method of claim 99, wherein the breast cancer is an estrogen receptor negative, progesterone receptor negative, human epidermal growth factor receptor 2 (HER2) positive metastatic breast cancer.

146. The method of claim 99, wherein the subject is a woman.

147. The method of claim 99, wherein the subject is a postmenopausal woman.

148. The method of claim 99, wherein the subject is a man.

149. The method of claim 99, wherein the subject received a therapy other than the compound for the breast cancer prior to the administering.

150. The method of claim 149, wherein the therapy was received after the subject was diagnosed with breast cancer.

151. The method of claim 149, wherein the subject has not responded to the therapy.

152. The method of claim 149, wherein the subject experienced a relapse of the breast cancer after the therapy.

153. The method of claim 99, wherein the administering is oral.

154. The method of claim 99 wherein the administering is intravenous.

155. The method of claim 99, wherein the administering is oral on a 4-week cycle of:
(i) a continuous, three-week period of once-daily, morning administration, wherein the therapeutically-effective amount is from about 80 mg to about 500 mg; and
(ii) immediately following the three-week period, one week of no administration.

156. The method of claim 99, wherein the compound is administered in a pharmaceutical composition, wherein the pharmaceutical composition is in a unit dosage form, the unit dosage form further comprising a pharmaceutically-acceptable excipient.

157. The method of claim 156, wherein the unit dosage form comprises about 40 mg of 8-cyclopentyl-2-((4-(4-methylpiperazin-1-yl)phenyl)amino)-7-oxo-7,8-dihydropyrido[2,3-d]pyrimidine-6-carbonitrile.

158. The method of claim 156, wherein the unit dosage form comprises about 48.4 mg of 8-cyclopentyl-2-((4-(4-methylpiperazin-1-yl)phenyl)amino)-7-oxo-7,8-dihydropyrido[2,3-d]pyrimidine-6-carbonitrile monolactate.

159. The method of claim 156, wherein the unit dosage form is a capsule.

160. The method of claim 156, wherein the unit dosage form is a tablet.

161. The method of claim 99, wherein the administering occurs in a morning of a day.

162. The method of claim 99, wherein the subject is in a fasted state.

163. The method of claim 99, further comprising administering to the subject a therapeutically-effective amount of a second therapeutic agent.

164. The method of claim 163, wherein the second therapeutic agent is an estrogen receptor modulator.

165. The method of claim 163, wherein the second therapeutic agent is an estrogen receptor blocker.

166. The method of claim 163, wherein the second therapeutic agent is an aromatase inhibitor.

167. The method of claim 163, wherein the second therapeutic agent is an aromatase inhibitor, wherein the aromatase inhibitor is administered as an initial endocrine-based therapy.

168. The method of claim 163, wherein the second therapeutic agent is letrozole or a pharmaceutically-acceptable salt thereof.

169. The method of claim 168, wherein the letrozole or pharmaceutically-acceptable salt thereof is administered in a pharmaceutical composition, wherein the pharmaceutical composition is in a unit dosage form, the unit dosage form further comprising a pharmaceutically-acceptable excipient.

170. The method of claim 169, wherein the unit dosage form comprises about 2.5 mg of letrozole.

171. The method of claim 169, wherein the unit dosage form is a tablet.

172. The method of claim 163, wherein the second therapeutic agent is a selective estrogen receptor degrader.

173. The method of claim 172, wherein the selective estrogen receptor degrader is fulvestrant.

174. The method of claim 172, wherein the selective estrogen receptor degrader is fulvestrant, wherein the fulvestrant is administered following an endocrine therapy.

175. The method of claim 163, wherein the second therapeutic agent is a progestin.

176. The method of claim 175, wherein the progestin is megestrol acetate.

177. The method of claim 176, wherein megestrol acetate is administered in a pharmaceutical composition, wherein the pharmaceutical composition is in a unit dosage form, the unit dosage form further comprising a pharmaceutically-acceptable excipient.

178. The method of claim 177, wherein the unit dosage form comprises about 125 mg/mL of megestrol acetate.

179. The method of claim 177, wherein the unit dosage form is an oral suspension.

180. The method of claim 177, wherein the megestrol acetate is administered at a dose of about 625 mg per day.

181. The method of claim 163, wherein the administering of the second therapeutic agent is once daily.

182. The method of claim 163, wherein the second therapeutic agent is a vascular endothelial growth factor inhibitor.

183. The method of claim 163, wherein the second therapeutic agent is a phosphoinositide 3-kinase inhibitor.

184. The method of claim 163, wherein the administering of the second therapeutic agent is oral.

185. The method of claim 163, wherein the administering of the second therapeutic agent is intravenous.

186. The method of claim 163, wherein the administering of the second therapeutic agent is intramuscular.

187. The method of claim 163, wherein the administering of the compound of formula (I) is oral, and the administering of the second therapeutic agent is oral.

188. The method of claim 163, wherein the administering of the compound of formula (I) is oral, and the administering of the second therapeutic agent is intravenous.

189. The method of claim 163, wherein the administering of the compound of formula (I) is oral, and the administering of the second therapeutic agent is intramuscular.

190. The method of claim 163, wherein the administering of the compound of formula (I) is prior to the administering of the second therapeutic agent.

191. The method of claim 163, wherein the administering of the second therapeutic agent is prior to the administering of the compound of formula (I).

192. The method of claim 163, wherein the administering of the compound of formula (I) is concurrently with the administering of the second therapeutic agent.

193. A method of treating breast cancer in a subject in need thereof, the method comprising administering to the subject a therapeutically-effective amount of a compound of formula (I) 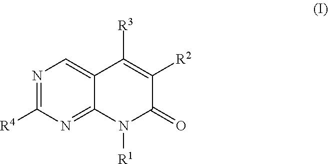
wherein:
R¹ is alkyl, alkenyl, alkynyl, cycloalkyl, aryl, heteroaryl or heterocyclyl, each of which is unsubstituted or substituted, or hydrogen;
R² is alkyl, alkenyl, alkynyl, cycloalkyl, aryl, heteroaryl, heterocyclyl, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
R³ is alkyl, alkenyl, alkynyl, cycloalkyl, aryl, heterocyclyl or heteroaryl, each of which is unsubstituted or substituted, or —CN, hydrogen, or halogen;
R⁴ is alkyl, alkenyl, alkynyl, cycloalkyl, aryl, heterocyclyl, heteroaryl, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
each R⁵ and R⁶ is independently alkyl, alkenyl, alkynyl, cycloalkyl, aryl, heterocyclyl, or heteroaryl, each of which is independently substituted or unsubstituted, or —CN, hydrogen, or halogen;
provided that, when R⁴ is —NR⁵R⁶ and one of R⁵ and R⁶ is pyridyl, then R² is not —C(O)CH₃,
or a pharmaceutically-acceptable salt thereof;
wherein the subject received a therapy other than the compound for the breast cancer prior to the administering.

194. The method of claim 193, wherein R¹ is cycloalkyl.

195. The method of claim 193, wherein R¹ is cyclopentyl.

196. The method of claim 193, wherein R² is CN.

197. The method of claim 193, wherein R³ is hydrogen.

198. The method of claim 193, wherein R⁴ is —NR⁵R⁶.

199. The method of claim 198, wherein one of R⁵ and R⁶ is hydrogen.

200. The method of claim 198, wherein one of R⁵ and R⁶ is phenyl.

201. The method of claim 198, wherein one of R⁵ and R⁶ is phenyl substituted with heterocyclyl.

202. The method of claim 198, wherein one of R⁵ and R⁶ is phenyl substituted with piperazinyl.

203. The method of claim 198, wherein one of R⁵ and R⁶ is phenyl substituted with 4-methyl piperazinyl.

204. The method of claim 193, wherein R⁴ is 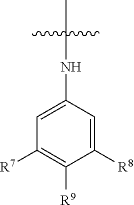
wherein:
R⁷ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen;
R⁸ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen; and
R⁹ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen.

205. The method of claim 204, wherein R⁷ is hydrogen.

206. The method of claim 204, wherein R⁸ is hydrogen.

207. The method of claim 204, wherein R⁹ is unsubstituted or substituted heterocyclyl.

208. The method of claim 204, wherein R⁹ is unsubstituted or substituted piperazinyl.

209. The method of claim 204, wherein R⁹ is 4-methyl piperazinyl.

210. The method of claim 204, wherein the compound is a compound of formula (II)

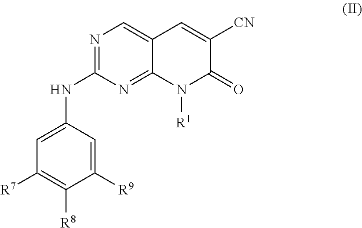

211. The method of claim 204, wherein the compound is a compound of formula (III) 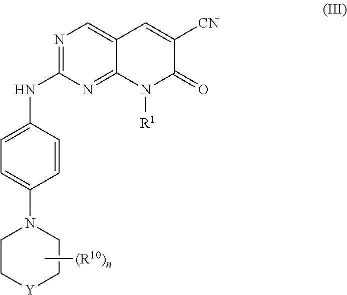
wherein:
Y is O, S, or NR¹¹;
each R¹⁰ is independently alkyl, alkenyl, alkynyl, cycloalkyl, aryl, heterocyclyl, heteroaryl, —OR⁵, —SR⁵, or —NR⁵R⁶, each of which is unsubstituted or substituted;
R¹¹ is alkyl, alkenyl, alkynyl, cycloalkyl, aryl, heterocyclyl, or heteroaryl, each of which is independently substituted or unsubstituted, or hydrogen or halogen; and
n is 0, 1, 2, 3, 4, 5, 6, 7, or 8.

212. The method of claim 211, wherein R¹ is cycloalkyl.

213. The method of claim 211, wherein R¹ is cyclopentyl.

214. The method of claim 211, wherein Y is NR¹¹.

215. The method of claim 214, wherein R¹¹ is alkyl.

216. The method of claim 214, wherein R¹¹ is methyl.

217. The method of claim 211, wherein n is 0.

218. The method of claim 193, wherein the compound is 8-cyclopentyl-2-((4-(4-methylpiperazin-1-yl)phenyl)amino)-7-oxo-7,8-dihydropyrido[2,3-d]pyrimidine-6-carbonitrile, or a pharmaceutically-acceptable salt thereof.

219. The method of claim 218, wherein the compound is the pharmaceutically-acceptable salt and the pharmaceutically-acceptable salt is monolactate.

220. The method of claim 193, wherein the therapeutically-effective amount is about 80 mg to about 500 mg per day.

221. The method of claim 193, wherein the breast cancer is a hormone receptor positive breast cancer.

222. The method of claim 193, wherein the breast cancer is an estrogen receptor positive breast cancer.

223. The method of claim 193, wherein the breast cancer is a progesterone receptor positive breast cancer.

224. The method of claim 193, wherein the breast cancer is an estrogen receptor positive, progesterone receptor positive breast cancer.

225. The method of claim 193, wherein the breast cancer is a human epidermal growth factor receptor 2 (HER2) positive breast cancer.

226. The method of claim 193, wherein the breast cancer is a hormone receptor positive, human epidermal growth factor receptor 2 (HER2) positive breast cancer.

227. The method of claim 193, wherein the breast cancer is an estrogen receptor positive, progesterone receptor positive, human epidermal growth factor receptor 2 (HER2) positive breast cancer.

228. The method of claim 193, wherein the breast cancer is a hormone receptor positive breast cancer.

229. The method of claim 193, wherein the breast cancer is an estrogen receptor positive breast cancer.

230. The method of claim 193, wherein the breast cancer is a progesterone receptor positive breast cancer.

231. The method of claim 193, wherein breast cancer is a hormone receptor negative, human epidermal growth factor receptor 2 (HER2) positive breast cancer.

232. The method of claim 193, wherein breast cancer is an estrogen receptor negative, progesterone receptor negative, human epidermal growth factor receptor 2 (HER2) positive breast cancer.

233. The method of claim 232, wherein the breast cancer expresses Mitotic checkpoint serine/threonine-protein kinase BUB1.

234. The method of claim 193, wherein the breast cancer is advanced breast cancer.

235. The method of claim 193, wherein the breast cancer is metastatic breast cancer.

236. The method of claim 193, wherein the breast cancer is a hormone receptor positive, human epidermal growth factor receptor 2 (HER2) positive metastatic breast cancer.

237. The method of claim 193, wherein the breast cancer is an estrogen receptor positive, progesterone receptor positive, human epidermal growth factor receptor 2 (HER2) positive metastatic breast cancer.

238. The method of claim 193, wherein the breast cancer is hormone receptor negative, human epidermal growth factor receptor 2 (HER2) positive metastatic breast cancer.

239. The method of claim 193, wherein the breast cancer is an estrogen receptor negative, progesterone receptor negative, human epidermal growth factor receptor 2 (HER2) positive metastatic breast cancer.

240. The method of claim 193, wherein the subject is a woman.

241. The method of claim 193, wherein the subject is a postmenopausal woman.

242. The method of claim 193, wherein the subject is a man.

243. The method of claim 193, wherein the therapy was received after the subject was diagnosed with the breast cancer.

244. The method of claim 193, wherein the subject has not responded to the therapy.

245. The method of claim 193, wherein the subject experienced a relapse of the breast cancer after the therapy.

246. The method of claim 193, wherein the administering is oral.

247. The method of claim 193, wherein the administering is intravenous.

248. The method of claim 193, wherein the administering is once daily.

249. The method of claim 193, wherein the administering is once daily for at least 1 week.

250. The method of claim 193, wherein the administering is once daily for at least 2 weeks.

251. The method of claim 193, wherein the administering is once daily for at least 4 weeks.

252. The method of claim 193, wherein the administering is oral on a 4-week cycle of:
(i) a continuous, three-week period of once-daily administration; and
(ii) immediately following the three-week period, one week of no administration.

253. The method of claim 193, wherein the administering is oral on a 4-week cycle of:
(i) a continuous, three-week period of once-daily, morning administration, wherein the therapeutically-effective amount is from about 40 mg to about 500 mg; and
(ii) immediately following the three-week period, one week of no administration.

254. The method of claim 193, wherein the compound is administered in a pharmaceutical composition, wherein the pharmaceutical composition is in a unit dosage form, the unit dosage form further comprising a pharmaceutically-acceptable excipient.

255. The method of claim 254, wherein the unit dosage form comprises about 40 mg of 8-cyclopentyl-2-((4-(4-methylpiperazin-1-yl)phenyl)amino)-7-oxo-7,8-dihydropyrido[2,3-d]pyrimidine-6-carbonitrile.

256. The method of claim 254, wherein the unit dosage form comprises about 48.4 mg of 8-cyclopentyl-2-((4-(4-methylpiperazin-1-yl)phenyl)amino)-7-oxo-7,8-dihydropyrido[2,3-d]pyrimidine-6-carbonitrile monolactate.

257. The method of claim 254, wherein the unit dosage form is a capsule.

258. The method of claim 254, wherein the unit dosage form is a tablet.

259. The method of claim 193, wherein the administering occurs in a morning of a day.

260. The method of claim 193, wherein the subject is in a fasted state.

261. The method of claim 193, further comprising administering to the subject a therapeutically-effective amount of a second therapeutic agent.

262. The method of claim 261, wherein the second therapeutic agent is an estrogen receptor modulator.

263. The method of claim 261, wherein the second therapeutic agent is an estrogen receptor blocker.

264. The method of claim 261, wherein the second therapeutic agent is an aromatase inhibitor.

265. The method of claim 261, wherein the second therapeutic agent is an aromatase inhibitor, wherein the aromatase inhibitor is administered as an initial endocrine-based therapy.

266. The method of claim 261, wherein the second therapeutic agent is letrozole or a pharmaceutically-acceptable salt thereof.

267. The method of claim 261, wherein the letrozole or a pharmaceutically-acceptable salt thereof is administered in a pharmaceutical composition, wherein the pharmaceutical composition is in a unit dosage form, the unit dosage form further comprising a pharmaceutically-acceptable excipient.

268. The method of claim 267, wherein the unit dosage form comprises about 2.5 mg of letrozole.

269. The method of claim 267, wherein the unit dosage form is a tablet.

270. The method of claim 261, wherein the second therapeutic agent is a selective estrogen receptor degrader.

271. The method of claim 270, wherein the selective estrogen receptor degrader is fulvestrant.

272. The method of claim 270, wherein the selective estrogen receptor degrader is fulvestrant, wherein the fulvestrant is administered when the breast cancer progresses following an endocrine therapy.

273. The method of claim 261, wherein the second therapeutic agent is a progestin.

274. The method of claim 273, wherein the progestin is megestrol acetate.

275. The method of claim 274, wherein megestrol acetate is administered in a pharmaceutical composition, wherein the pharmaceutical composition is in a unit dosage form, the unit dosage form further comprising a pharmaceutically-acceptable excipient.

276. The method of claim 275, wherein the unit dosage form comprises about 125 mg/mL of megestrol acetate.

277. The method of claim 275, wherein the unit dosage form is an oral suspension.

278. The method of claim 275, wherein the megestrol acetate is administered at a dose of about 625 mg per day.

279. The method of claim 261, wherein the administering of the second therapeutic agent is once daily.

280. The method of claim 261, wherein the second therapeutic agent is a vascular endothelial growth factor inhibitor.

281. The method of claim 261, wherein the second therapeutic agent is a phosphoinositide 3-kinase inhibitor.

282. The method of claim 261, wherein the administering of the second therapeutic agent is oral.

283. The method of claim 261, wherein the administering of the second therapeutic agent is intravenous.

284. The method of claim 261, wherein the administering of the second therapeutic agent is intramuscular.

285. The method of claim 261, wherein the administering of the compound of formula (I) is oral, and the administering of the second therapeutic agent is oral.

286. The method of claim 261, wherein the administering of the compound of formula (I) is oral, and the administering of the second therapeutic agent is intravenous.

287. The method of claim 261, wherein the administering of the compound of formula (I) is oral, and the administering of the second therapeutic agent is intramuscular.

288. The method of claim 261, wherein the administering of the compound of formula (I) is prior to the administering of the second therapeutic agent.

289. The method of claim 261, wherein the administering of the second therapeutic agent is prior to the administering of the compound of formula (I).

290. The method of claim 261, wherein the administering of the compound of formula (I) is concurrently with the administering of the second therapeutic agent.

291. A method of treating breast cancer in a subject in need thereof, the method comprising:
(i) administering to the subject a therapeutically-effective amount of a first compound, wherein the first compound is a compound of formula (I): 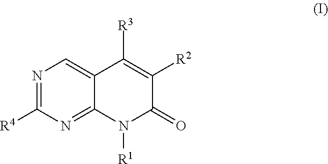
wherein:
R¹ is alkyl, alkenyl, alkynyl, cycloalkyl, aryl, heteroaryl or heterocyclyl, each of which is unsubstituted or substituted, or hydrogen;
R² is alkyl, alkenyl, alkynyl, cycloalkyl, aryl, heteroaryl, heterocyclyl, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
R³ is alkyl, alkenyl, alkynyl, cycloalkyl, aryl, heterocyclyl or heteroaryl, each of which is unsubstituted or substituted, or —CN, hydrogen, or halogen;
R⁴ is alkyl, alkenyl, alkynyl, cycloalkyl, aryl, heterocyclyl, heteroaryl, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
each R⁵ and R⁶ is independently alkyl, alkenyl, alkynyl, cycloalkyl, aryl, heterocyclyl, or heteroaryl, each of which is independently substituted or unsubstituted, or —CN, hydrogen, or halogen;
provided that, when R⁴ is —NR⁵R⁶ and one of R⁵ and R⁶ is pyridyl, then R² is not —C(O)CH₃,
or a pharmaceutically-acceptable salt thereof; and
(ii) administering to the subject a therapeutically-effective amount of a second therapeutic agent.

292. The method of claim 291, wherein R¹ is cycloalkyl.

293. The method of claim 291, wherein R¹ is cyclopentyl.

294. The method of claim 291, wherein R² is CN.

295. The method of claim 291, wherein R³ is hydrogen.

296. The method of claim 291, wherein R⁴ is —NR⁵R⁶.

297. The method of claim 296, wherein one of R⁵ and R⁶ is hydrogen.

298. The method of claim 296, wherein one of R⁵ and R⁶ is phenyl.

299. The method of claim 296, wherein one of R⁵ and R⁶ is phenyl substituted with heterocyclyl.

300. The method of claim 296, wherein one of R⁵ and R⁶ is phenyl substituted with piperazinyl.

301. The method of claim 296, wherein one of R⁵ and R⁶ is phenyl substituted with 4-methyl piperazinyl.

302. The method of claim 291, wherein R⁴ is 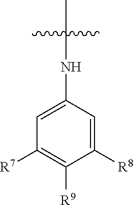
wherein:
R⁷ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen;
R⁸ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen; and
R⁹ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen.

303. The method of claim 302, wherein R⁷ is hydrogen.

304. The method of claim 302, wherein R⁸ is hydrogen.

305. The method of claim 302, wherein R⁹ is unsubstituted or substituted heterocyclyl.

306. The method of claim 302, wherein R⁹ is unsubstituted or substituted piperazinyl.

307. The method of claim 302, wherein R⁹ is 4-methyl piperazinyl.

308. The method of claim 302, wherein the compound is a compound of formula (II)

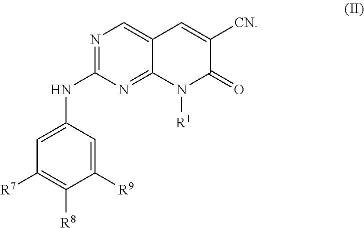

309. The method of claim 302, wherein the compound is a compound of formula (III) 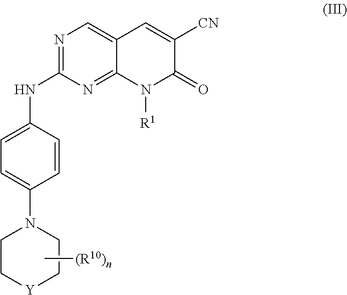
wherein:
Y is O, S, or NR¹¹;
each R¹⁰ is independently alkyl, alkenyl, alkynyl, cycloalkyl, aryl, heterocyclyl, heteroaryl, —OR⁵, —SR⁵, or —NR⁵R⁶, each of which is unsubstituted or substituted;
R¹¹ is alkyl, alkenyl, alkynyl, cycloalkyl, aryl, heterocyclyl, or heteroaryl, each of which is independently substituted or unsubstituted, or hydrogen or halogen; and
n is 0, 1, 2, 3, 4, 5, 6, 7, or 8.

310. The method of claim 309, wherein R¹ is cycloalkyl.

311. The method of claim 309, wherein R¹ is cyclopentyl.

312. The method of claim 309, wherein Y is NR¹¹.

313. The method of claim 312, wherein R¹¹ is alkyl.

314. The method of claim 312, wherein R¹¹ is methyl.

315. The method of claim 309, wherein n is 0.

316. The method of claim 291, wherein the first compound is 8-cyclopentyl-2-((4-(4-methylpiperazin-1-yl)phenyl)amino)-7-oxo-7,8-dihydropyrido[2,3-d]pyrimidine-6-carbonitrile, or a pharmaceutically-acceptable salt thereof.

317. The method of claim 316, wherein the compound is the pharmaceutically-acceptable salt and the pharmaceutically-acceptable salt is monolactate.

318. The method of claim 291, wherein the therapeutically-effective amount is about 40 mg to about 500 mg per day.

319. The method of claim 291, wherein the breast cancer is a hormone receptor positive breast cancer.

320. The method of claim 291, wherein the breast cancer is an estrogen receptor positive breast cancer.

321. The method of claim 291, wherein the breast cancer is a progesterone receptor positive breast cancer.

322. The method of claim 291, wherein the breast cancer is an estrogen receptor positive, progesterone receptor positive breast cancer.

323. The method of claim 291, wherein the breast cancer is a human epidermal growth factor receptor 2 (HER2) positive breast cancer.

324. The method of claim 291, wherein the breast cancer is a hormone receptor positive, human epidermal growth factor receptor 2 (HER2) positive breast cancer.

325. The method of claim 291, wherein the breast cancer is an estrogen receptor positive, progesterone receptor positive, human epidermal growth factor receptor 2 (HER2) positive breast cancer.

326. The method of claim 291, wherein the breast cancer is a hormone receptor positive breast cancer.

327. The method of claim 291, wherein the breast cancer is an estrogen receptor positive breast cancer.

328. The method of claim 291, wherein the breast cancer is a progesterone receptor positive breast cancer.

329. The method of claim 291, wherein breast cancer is a hormone receptor negative, human epidermal growth factor receptor 2 (HER2) positive breast cancer.

330. The method of claim 291, wherein breast cancer is an estrogen receptor negative, progesterone receptor negative, human epidermal growth factor receptor 2 (HER2) positive breast cancer.

331. The method of claim 330, wherein the breast cancer expresses Mitotic checkpoint serine/threonine-protein kinase BUB1.

332. The method of claim 291, wherein the breast cancer is advanced breast cancer.

333. The method of claim 291, wherein the breast cancer is metastatic breast cancer.

334. The method of claim 291, wherein the breast cancer is a hormone receptor positive, human epidermal growth factor receptor 2 (HER2) positive metastatic breast cancer.

335. The method of claim 291, wherein the breast cancer is an estrogen receptor positive, progesterone receptor positive, human epidermal growth factor receptor 2 (HER2) positive metastatic breast cancer.

336. The method of claim 291, wherein the breast cancer is hormone receptor negative, human epidermal growth factor receptor 2 (HER2) positive metastatic breast cancer.

337. The method of claim 291, wherein the breast cancer is an estrogen receptor negative, progesterone receptor negative, human epidermal growth factor receptor 2 (HER2) positive metastatic breast cancer.

338. The method of claim 291, wherein the subject is a woman.

339. The method of claim 291, wherein the subject is a postmenopausal woman.

340. The method of claim 291, wherein the subject is a man.

341. The method of claim 291, wherein the subject received a therapy other than the compound for the breast cancer prior to the administering of the first compound, and prior to the administering of the second therapeutic agent.

342. The method of claim 341, wherein the therapy was received after the subject was diagnosed with breast cancer.

343. The method of claim 341, wherein the subject has not responded to the therapy.

344. The method of claim 341, wherein the subject experienced a relapse of the breast cancer after the therapy.

345. The method of claim 291, wherein the administering of the first compound is oral.

346. The method of claim 291, wherein the administering of the first compound is intravenous.

347. The method of claim 291, wherein the administering of the first compound is once daily.

348. The method of claim 291, wherein the administering of the first compound is once daily for at least 4 weeks.

349. The method of claim 291, wherein the administering of the first compound is oral on a 4-week cycle of:
(i) a continuous, three-week period of once-daily administration; and
(ii) immediately following the three-week period, one week of no administration.

350. The method of claim 291, wherein the administering of the first compound is oral on a 4-week cycle of:
(i) a continuous, three-week period of once-daily, morning administration, wherein the therapeutically-effective amount is from about 40 mg to about 500 mg; and
(ii) immediately following the three-week period, one week of no administration.

351. The method of claim 291, wherein the first compound is administered in a pharmaceutical composition, wherein the pharmaceutical composition is in a unit dosage form, the unit dosage form further comprising a pharmaceutically-acceptable excipient.

352. The method of claim 351, wherein the unit dosage form comprises about 40 mg of 8-cyclopentyl-2-((4-(4-methylpiperazin-1-yl)phenyl)amino)-7-oxo-7,8-dihydropyrido[2,3-d]pyrimidine-6-carbonitrile.

353. The method of claim 351, wherein the unit dosage form comprises about 48.4 mg of 8-cyclopentyl-2-((4-(4-methylpiperazin-1-yl)phenyl)amino)-7-oxo-7,8-dihydropyrido[2,3-d]pyrimidine-6-carbonitrile monolactate.

354. The method of claim 351, wherein the unit dosage form is a capsule.

355. The method of claim 291, wherein the administering occurs in a morning of a day.

356. The method of claim 291, wherein the subject is in a fasted state.

357. The method of claim 291, wherein the second therapeutic agent is an estrogen receptor modulator.

358. The method of claim 291, wherein the second therapeutic agent is an estrogen receptor blocker.

359. The method of claim 291, wherein the second therapeutic agent is an aromatase inhibitor.

360. The method of claim 359, wherein the second therapeutic agent is an aromatase inhibitor, wherein the aromatase inhibitor is administered as an initial endocrine-based therapy.

361. The method of claim 291, wherein the second therapeutic agent is letrozole or a pharmaceutically-acceptable salt thereof.

362. The method of claim 291, wherein the letrozole or pharmaceutically-acceptable salt thereof is administered in a pharmaceutical composition, wherein the pharmaceutical composition is in a unit dosage form, the unit dosage form further comprising a pharmaceutically-acceptable excipient.

363. The method of claim 362, wherein the unit dosage form comprises about 2.5 mg of letrozole.

364. The method of claim 362, wherein the unit dosage form is a tablet.

365. The method of claim 291, wherein the second therapeutic agent is a selective estrogen receptor degrader.

366. The method of claim 365, wherein the selective estrogen receptor degrader is fulvestrant.

367. The method of claim 365, wherein the selective estrogen receptor degrader is fulvestrant, wherein the fulvestrant is administered when the breast cancer progresses following an endocrine therapy.

368. The method of claim 291, wherein the second therapeutic agent is a progestin.

369. The method of claim 368, wherein the progestin is megestrol acetate.

370. The method of claim 369, wherein megestrol acetate is administered in a pharmaceutical composition, wherein the pharmaceutical composition is in a unit dosage form, the unit dosage form further comprising a pharmaceutically-acceptable excipient.

371. The method of claim 370, wherein the unit dosage form comprises about 125 mg/mL of megestrol acetate.

372. The method of claim 370, wherein the unit dosage form is an oral suspension.

373. The method of claim 370, wherein the megestrol acetate is administered at a dose of about 625 mg per day.

374. The method of claim 291, wherein the administering of the second therapeutic agent is once daily.

375. The method of claim 291, wherein the second therapeutic agent is a vascular endothelial growth factor inhibitor.

376. The method of claim 291, wherein the second therapeutic agent is a phosphoinositide 3-kinase inhibitor.

377. The method of claim 291, wherein the administering of the second therapeutic agent is oral.

378. The method of claim 291, wherein the administering of the second therapeutic agent is intravenous.

379. The method of claim 291, wherein the administering of the second therapeutic agent is intramuscular.

380. The method of claim 291, wherein the administering of the first compound is oral, and the administering of the second therapeutic agent is oral.

381. The method of claim 291, wherein the administering of the compound of formula (I) is oral, and the administering of the second therapeutic agent is intravenous.

382. The method of claim 291, wherein the administering of the compound of formula (I) is oral, and the administering of the second therapeutic agent is intramuscular.

383. The method of claim 291, wherein the administering of the first compound is prior to the administering of the second therapeutic agent.

384. The method of claim 291, wherein the administering of the second therapeutic agent is prior to the administering of the first compound.

385. The method of claim 291, wherein the administering of the first compound is concurrently with the administering of the second therapeutic agent.

386. A combination comprising:
(i) a compound of formula (I): 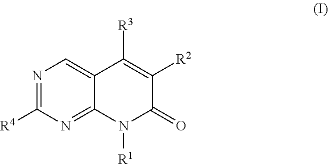
wherein:
R¹ is alkyl, alkenyl, alkynyl, cycloalkyl, aryl, heteroaryl or heterocyclyl, each of which is unsubstituted or substituted, or hydrogen;
R² is alkyl, alkenyl, alkynyl, cycloalkyl, aryl, heteroaryl, heterocyclyl, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or —CN, hydrogen, or halogen;
R³ is C₂-C₆ alkyl, alkenyl, alkynyl, cycloalkyl, aryl, heterocyclyl or heteroaryl, each of which is unsubstituted or substituted, or hydrogen or halogen;
R⁴ is alkyl, alkenyl, alkynyl, cycloalkyl, aryl, heterocyclyl, heteroaryl, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or —CN, hydrogen, or halogen; and
each R⁵ and R⁶ is independently alkyl, alkenyl, alkynyl, cycloalkyl, aryl, heterocyclyl, or heteroaryl, each of which is independently substituted or unsubstituted, or hydrogen or halogen,
or a pharmaceutically-acceptable salt thereof; and
(ii) an aromatase inhibitor.

387. The combination of claim 386, wherein R¹ is cycloalkyl.

388. The combination of claim 386, wherein R¹ is cyclopentyl.

389. The combination of claim 386, wherein R² is CN.

390. The combination of claim 386, wherein R³ is hydrogen.

391. The combination of claim 386, wherein R⁴ is —NR⁵R⁶.

392. The combination of claim 391, wherein one of R⁵ and R⁶ is hydrogen.

393. The combination of claim 391, wherein one of R⁵ and R⁶ is phenyl.

394. The combination of claim 391, wherein one of R⁵ and R⁶ is phenyl substituted with heterocyclyl.

395. The combination of claim 391, wherein one of R⁵ and R⁶ is phenyl substituted with piperazinyl.

396. The combination of claim 391, wherein one of R⁵ and R⁶ is phenyl substituted with 4-methyl piperazinyl.

397. The combination of claim 386, wherein R⁴ is 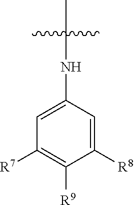
wherein:
R⁷ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen;
R⁸ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen; and
R⁹ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen.

398. The combination of claim 397, wherein R⁷ is hydrogen.

399. The combination of claim 397, wherein R⁸ is hydrogen.

400. The combination of claim 397, wherein R⁹ is unsubstituted or substituted heterocyclyl.

401. The combination of claim 397, wherein R⁹ is unsubstituted or substituted piperazinyl.

402. The combination of claim 397, wherein R⁹ is 4-methyl piperazinyl.

403. The combination of claim 397, wherein the compound is a compound of formula (II)

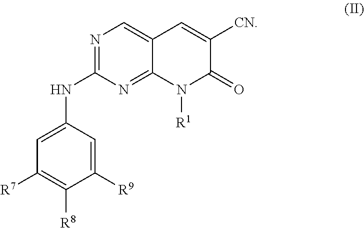

404. The combination of claim 397, wherein the compound is a compound of formula (III) 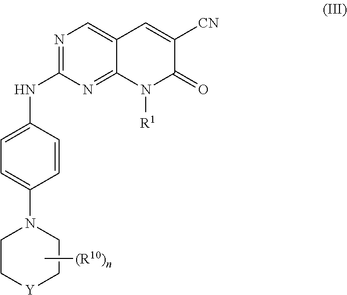
wherein:
Y is O, S, or NR¹¹;
each R¹⁰ is independently alkyl, alkenyl, alkynyl, cycloalkyl, aryl, heterocyclyl, heteroaryl, —OR⁵, —SR⁵, or —NR⁵R⁶, each of which is unsubstituted or substituted;
R¹¹ is alkyl, alkenyl, alkynyl, cycloalkyl, aryl, heterocyclyl, or heteroaryl, each of which is independently substituted or unsubstituted, or hydrogen or halogen; and
n is 0, 1, 2, 3, 4, 5, 6, 7, or 8.

405. The combination of claim 404, wherein R¹ is cycloalkyl.

406. The combination of claim 404, wherein R¹ is cyclopentyl.

407. The combination of claim 404, wherein Y is NR¹¹.

408. The combination of claim 407, wherein R¹¹ is alkyl.

409. The combination of claim 407, wherein R¹¹ is methyl.

410. The combination of claim 404, wherein n is 0.

411. The combination of claim 386, wherein the compound is 8-cyclopentyl-2-((4-(4-methylpiperazin-1-yl)phenyl)amino)-7-oxo-7,8-dihydropyrido[2,3-d]pyrimidine-6-carbonitrile, or a pharmaceutically-acceptable salt thereof.

412. The combination of claim 411, wherein the compound is the pharmaceutically-acceptable salt and the pharmaceutically-acceptable salt is monolactate.

413. The combination of claim 386, wherein the compound is in a pharmaceutical composition, wherein the pharmaceutical composition is in a unit dosage form, the unit dosage form further comprising a pharmaceutically-acceptable excipient.

414. The combination of claim 413, wherein the unit dosage form comprises about 40 mg of 8-cyclopentyl-2-((4-(4-methylpiperazin-1-yl)phenyl)amino)-7-oxo-7,8-dihydropyrido[2,3-d]pyrimidine-6-carbonitrile.

415. The combination of claim 413, wherein the unit dosage form comprises about 48.4 mg of 8-cyclopentyl-2-((4-(4-methylpiperazin-1-yl)phenyl)amino)-7-oxo-7,8-dihydropyrido[2,3-d]pyrimidine-6-carbonitrile monolactate.

416. The combination of claim 413, wherein the unit dosage form is a capsule.

417. The combination of claim 386, wherein the compound is formulated for oral administration.

418. The combination of claim 386, wherein the compound is formulated for intravenous administration.

419. The combination of claim 386, wherein the aromatase inhibitor is letrozole or a pharmaceutically-acceptable salt thereof.

420. The combination of claim 419, wherein the letrozole or a pharmaceutically-acceptable salt thereof is in a pharmaceutical composition, wherein the pharmaceutical composition is in a unit dosage form, the unit dosage form further comprising a pharmaceutically-acceptable excipient.

421. The combination of claim 420, wherein the unit dosage form is a tablet.

422. The combination of claim 420, wherein the unit dosage form comprises about 2.5 mg of letrozole.

423. The combination of claim 386, wherein the aromatase inhibitor is formulated for oral administration.

424. The combination of claim 386, wherein the aromatase inhibitor is formulated for intravenous administration.

425. The combination of claim 386, wherein the aromatase inhibitor is formulated for intramuscular administration.

426. The combination of claim 386, wherein the compound is formulated for oral administration, and the aromatase inhibitor is formulated for oral administration.

427. The combination of claim 386, wherein the compound is formulated for oral administration, and the aromatase inhibitor is formulated for intravenous administration.

428. The combination of claim 386, wherein the compound is formulated for oral administration, and the aromatase inhibitor is formulated for intramuscular administration.

429. The combination of claim 386, wherein the compound or the pharmaceutically acceptable salt thereof and the aromatase inhibitor are in one unit dosage form.

430. The combination of claim 386, wherein the compound or the pharmaceutically acceptable salt thereof and the aromatase inhibitor are in separate dosage forms.

431. A combination comprising:
(i) a compound of formula (I): 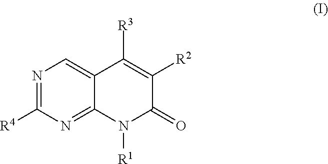
wherein:
R¹ is alkyl, alkenyl, alkynyl, cycloalkyl, aryl, heteroaryl or heterocyclyl, each of which is unsubstituted or substituted, or hydrogen;
R² is alkyl, alkenyl, alkynyl, cycloalkyl, aryl, heteroaryl, heterocyclyl, —C(O)R⁵, —C(O)OR⁵, —C(O)NR⁵R⁶, —C(═N)NR⁵R⁶, —OR⁵, —SR⁵, —NR⁵R⁶, —NRSC(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or —CN, hydrogen, or halogen;
R³ is C₂-C₆ alkyl, alkenyl, alkynyl, cycloalkyl, aryl, heterocyclyl or heteroaryl, each of which is unsubstituted or substituted, or hydrogen or halogen;
R⁴ is alkyl, alkenyl, alkynyl, cycloalkyl, aryl, heterocyclyl, heteroaryl, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or —CN, hydrogen, or halogen; and
each R⁵ and R⁶ is independently alkyl, alkenyl, alkynyl, cycloalkyl, aryl, heterocyclyl, or heteroaryl, each of which is independently substituted or unsubstituted, or hydrogen or halogen,
or a pharmaceutically-acceptable salt thereof; and
(ii) a selective estrogen receptor degrader.

432. The combination of claim 431, wherein R¹ is cycloalkyl.

433. The combination of claim 431, wherein R¹ is cyclopentyl.

434. The combination of claim 431, wherein R² is CN.

435. The combination of claim 431, wherein R³ is hydrogen.

436. The combination of claim 431, wherein R⁴ is —NR⁵R⁶.

437. The combination of claim 436, wherein one of R⁵ and R⁶ is hydrogen.

438. The combination of claim 436, wherein one of R⁵ and R⁶ is phenyl.

439. The combination of claim 436, wherein one of R⁵ and R⁶ is phenyl substituted with heterocyclyl.

440. The combination of claim 436, wherein one of R⁵ and R⁶ is phenyl substituted with piperazinyl.

441. The combination of claim 436, wherein one of R⁵ and R⁶ is phenyl substituted with 4-methyl piperazinyl.

442. The combination of claim 431, wherein R⁴ is 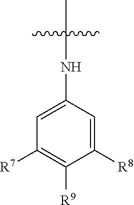
wherein:
R⁷ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen;
R⁸ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen; and
R⁹ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen.

443. The combination of claim 442, wherein R⁷ is hydrogen.

444. The combination of claim 442, wherein R⁸ is hydrogen.

445. The combination of claim 442, wherein R⁹ is unsubstituted or substituted heterocyclyl.

446. The combination of claim 442, wherein R⁹ is unsubstituted or substituted piperazinyl.

447. The combination of claim 442, wherein R⁹ is 4-methyl piperazinyl.

448. The combination of claim 442, wherein the compound is a compound of formula (II)

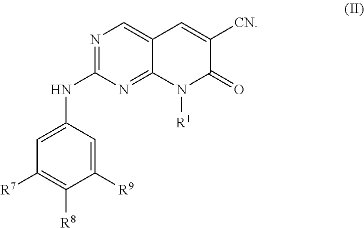

449. The combination of claim 431, wherein the compound is a compound of formula (III) 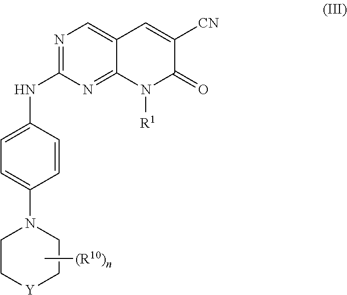
wherein:
Y is O, S, or NR¹¹;
each R¹⁰ is independently alkyl, alkenyl, alkynyl, cycloalkyl, aryl, heterocyclyl, heteroaryl, —OR⁵, —SR⁵, or —NR⁵R⁶, each of which is unsubstituted or substituted;
R¹¹ is alkyl, alkenyl, alkynyl, cycloalkyl, aryl, heterocyclyl, or heteroaryl, each of which is independently substituted or unsubstituted, or hydrogen or halogen; and
n is 0, 1, 2, 3, 4, 5, 6, 7, or 8.

450. The combination of claim 449, wherein R¹ is cycloalkyl.

451. The combination of claim 449, wherein R¹ is cyclopentyl.

452. The combination of claim 449, wherein Y is NR¹¹.

453. The combination of claim 452, wherein R¹¹ is alkyl.

454. The combination of claim 452, wherein R¹¹ is methyl.

455. The combination of claim 449, wherein n is 0.

456. The combination of claim 431, wherein the compound is 8-cyclopentyl-2-((4-(4-methylpiperazin-1-yl)phenyl)amino)-7-oxo-7,8-dihydropyrido[2,3-d]pyrimidine-6-carbonitrile, or a pharmaceutically-acceptable salt thereof.

457. The combination of claim 456, wherein the compound is the pharmaceutically-acceptable salt and the pharmaceutically-acceptable salt is monolactate.

458. The combination of claim 431, wherein the compound is in a pharmaceutical composition, wherein the pharmaceutical composition is in a unit dosage form, the unit dosage form further comprising a pharmaceutically-acceptable excipient.

459. The combination of claim 458, wherein the unit dosage form comprises about 40 mg of 8-cyclopentyl-2-((4-(4-methylpiperazin-1-yl)phenyl)amino)-7-oxo-7,8-dihydropyrido[2,3-d]pyrimidine-6-carbonitrile.

460. The combination of claim 458, wherein the unit dosage form comprises about 48.4 mg of 8-cyclopentyl-2-((4-(4-methylpiperazin-1-yl)phenyl)amino)-7-oxo-7,8-dihydropyrido[2,3-d]pyrimidine-6-carbonitrile monolactate.

461. The combination of claim 458, wherein the unit dosage form is a capsule.

462. The combination of claim 431, wherein the compound is formulated for oral administration.

463. The combination of claim 431, wherein the compound is formulated for intravenous administration.

464. The combination of claim 431, wherein the selective estrogen receptor degrader is fulvestrant.

465. The combination of claim 464, wherein the fulvestrant is in a pharmaceutical composition, wherein the pharmaceutical composition is in a unit dosage form, the unit dosage form further comprising a pharmaceutically-acceptable excipient.

466. The combination of claim 464, wherein the unit dosage form is an injectable solution.

467. The combination of claim 464, wherein the fulvestrant is formulated for intramuscular injection.

468. The combination of claim 431, wherein the compound is formulated for oral administration, and the selective estrogen receptor degrader is formulated for intramuscular administration.

469. The combination of claim 431, wherein the compound and the selective estrogen receptor degrader are in a common unit dosage form.

470. The combination of claim 431, wherein the compound and the selective estrogen receptor degrader are in separate dosage forms.

471. A method of treating breast cancer in a subject in need thereof, the method comprising orally administering to the subject a solid pharmaceutical composition, the solid pharmaceutical composition comprising 40 mg to 500 mg of a compound that is 8-cyclopentyl-2-((4-(4-methylpiperazin-1-yl)phenyl)amino)-7-oxo-7,8-dihydropyrido[2,3-d]pyrimidine-6-carbonitrile monolactate,
wherein the subject received a therapy other than the compound for the breast cancer prior to the administering, wherein the therapy was received after the subject was diagnosed with breast cancer, and wherein the subject has not responded to the therapy prior to the administering; and
wherein the administering comprises 3 weeks of once-daily administration.

472. A method of treating estrogen receptor positive, progesterone receptor positive, human epidermal growth factor 2 (HER)2 positive breast cancer in a subject in need thereof, the method comprising orally administering to the subject a solid pharmaceutical composition, the solid pharmaceutical composition comprising 40 mg to 500 mg of a compound that is 8-cyclopentyl-2-((4-(4-methylpiperazin-1-yl)phenyl)amino)-7-oxo-7,8-dihydropyrido[2,3-d]pyrimidine-6-carbonitrile monolactate,
wherein the subject received a therapy other than the compound for the breast cancer prior to the administering, wherein the therapy was received after the subject was diagnosed with breast cancer, and wherein the subject has not responded to the therapy prior to the administering; and
wherein the administering comprises 3 weeks of once-daily administration.

473. A method of treating estrogen receptor negative, progesterone receptor negative, human epidermal growth factor 2 (HER)2 positive breast cancer in a subject in need thereof, the method comprising orally administering to the subject a solid pharmaceutical composition, the solid pharmaceutical composition comprising 40 mg to 500 mg of a compound that is 8-cyclopentyl-2-((4-(4-methylpiperazin-1-yl)phenyl)amino)-7-oxo-7,8-dihydropyrido[2,3-d]pyrimidine-6-carbonitrile monolactate,
wherein the subject received a therapy other than the compound for the breast cancer prior to the administering, wherein the therapy was received after the subject was diagnosed with breast cancer, and wherein the subject has not responded to the therapy prior to the administering; and
wherein the administering comprises 3 weeks of once-daily administration.

474. A method of treating breast cancer in a subject in need thereof, the method comprising:
(i) orally administering to the subject a solid pharmaceutical composition, wherein the solid pharmaceutical composition comprises 40 mg to 500 mg of a compound that is 8-cyclopentyl-2-((4-(4-methylpiperazin-1-yl)phenyl)amino)-7-oxo-7,8-dihydropyrido[2,3-d]pyrimidine-6-carbonitrile monolactate,
wherein the subject received a therapy other than the compound for the breast cancer prior to the administering, wherein the therapy was received after the subject was diagnosed with breast cancer, wherein the subject has not responded to the therapy prior to the administering; and
wherein the administering comprises 3 weeks of once-daily administration; and
(ii) orally administering to the subject a therapeutically-effective amount of letrozole.

475. A method of treating breast cancer in a subject in need thereof, the method comprising:
(i) orally administering to the subject a solid pharmaceutical composition, wherein the solid pharmaceutical composition comprises 40 mg to 500 mg of a compound that is 8-cyclopentyl-2-((4-(4-methylpiperazin-1-yl)phenyl)amino)-7-oxo-7,8-dihydropyrido[2,3-d]pyrimidine-6-carbonitrile monolactate,
wherein the subject received a therapy other than the compound for the breast cancer prior to the administering, wherein the therapy was received after the subject was diagnosed with breast cancer, wherein the subject has not responded to the therapy prior to the administering, and
wherein the administering comprises 3 weeks of once-daily administration; and
(ii) orally administering to the subject a therapeutically-effective amount of fulvestrant.

476. The method of claim 1, wherein the breast cancer is a human epidermal growth factor receptor 2 (HER2) positive breast cancer.

477. The method of claim 1, wherein the breast cancer is a hormone receptor positive, human epidermal growth factor receptor 2 (HER2) positive breast cancer.

478. The method of claim 1, wherein the breast cancer is an estrogen receptor positive, progesterone receptor positive, human epidermal growth factor receptor 2 (HER2) positive breast cancer.

479. The method of claim 1, wherein the breast cancer is a hormone receptor positive breast cancer.

480. The method of claim 1, wherein the breast cancer is an estrogen receptor positive breast cancer.

481. The method of claim 1, wherein the breast cancer is a progesterone receptor positive breast cancer.

482. The method of claim 1, wherein breast cancer is a hormone receptor negative, human epidermal growth factor receptor 2 (HER2) positive breast cancer.

483. The method of claim 1, wherein the breast cancer is an estrogen receptor negative, progesterone receptor negative, human epidermal growth factor receptor 2 (HER2) negative breast cancer.

484. The method of claim 1, the breast cancer is an estrogen receptor positive, progesterone receptor positive, human epidermal growth factor receptor 2 (HER2) positive breast cancer.
